SEQUENCE LISTING (45 sequences)

SEQ ID NO: 1 - protein, 6 aa, synthetic construct
GFOGER

SEQ ID NO: 2 - protein, 5 aa, synthetic construct
PHSRN

SEQ ID NO: 3 - protein, 9 aa, synthetic construct
  MOD_RES 9: note = Amidated proline
GCRERGDSP

SEQ ID NO: 4 - protein, 9 aa, synthetic construct
  MOD_RES 1: note = Acetylated glycine
  MOD_RES 9: note = Amidated proline
GCRERGDSP

SEQ ID NO: 5 - protein, 31 aa, synthetic construct
  MOD_RES 31: note = Amidated proline
GCREISAFLGIPFAEPPMGPRRFLPPEPKKP

SEQ ID NO: 6 - protein, 16 aa, synthetic construct
GCRETLQPVYEYMVGV

SEQ ID NO: 7 - protein, 31 aa, synthetic construct; identical to SEQ ID NO: 5
  MOD_RES 1: note = Acetylated glycine
  MOD_RES 31: note = Amidated proline

SEQ ID NO: 8 - protein, 24 aa, synthetic construct
  MOD_RES 1: note = Acetylated proline
  MOD_RES 9: note = Acetylated lysine
  MOD_RES 24: note = Amidated tyrosine
PHSRNGGGKGGGERCGGGRGDSPY

SEQ ID NO: 9 - protein, 24 aa, synthetic construct
  MOD_RES 1: note = Acetylated proline
  MOD_RES 16: note = Acetylated glycine
  MOD_RES 24: note = Amidated tyrosine
PHSRNGGGKGGGERCGGGRDGSPY

SEQ ID NO: 10 - protein, 16 aa, synthetic construct
  MOD_RES 1: note = Acetylated glycine
  MOD_RES 16: note = Amidated glycine
GCRDGPQGIAGQDRCG

SEQ ID NO: 11 - protein, 47 aa, synthetic construct
  MOD_RES 47: note = Amidated Cysteine
GGYGGGPGGPPGPPGPPGPPGPPGFOGERGPPGPPGPPGPPGPPGPC

SEQ ID NO: 12 - protein, 47 aa, synthetic construct; identical to SEQ ID NO: 11
  MOD_RES 1: note = Acetylated glycine
  MOD_RES 47: note = Amidated Cysteine

SEQ ID NO: 13 - protein, 16 aa, synthetic construct
  MOD_RES 16: note = Amidated Arginine
GCRGPPFLMLLKGSTR

SEQ ID NO: 14 - protein, 16 aa, synthetic construct
  MOD_RES 1: note = Amidated glycine
  MOD_RES 16: note = Acetylated valine
GCRETLQPVYEYMVGV

SEQ ID NO: 15 - protein, 12 aa, synthetic construct
TLQPVYEYMVGV

SEQ ID NO: 16 - protein, 4 aa, synthetic construct
GCRE

SEQ ID NO: 17 - protein, 19 aa, synthetic construct
VPQIHGQNKGNQSFEEDTE

SEQ ID NO: 18 - protein, 19 aa, synthetic construct
VPQIQGQNKGNQSFEEDTE

SEQ ID NO: 19 - protein, 19 aa, synthetic construct
VPQIHGQNNGNQSFEEDTE

SEQ ID NO: 20 - protein, 19 aa, synthetic construct
VPQIQGQNNGNQSFEEDTE

SEQ ID NO: 21 - protein, 19 aa, synthetic construct
VPQIHGQNIGNQSFEEDTE

SEQ ID NO: 22 - protein, 19 aa, synthetic construct
VPQIQGQNIGNQSFEEDTE

SEQ ID NO: 23 - protein, 19 aa, synthetic construct
VPQIAGQNKGNQSFEEDTE

SEQ ID NO: 24 - protein, 19 aa, synthetic construct
VPQIAGQNAGNQSFEEDTE

SEQ ID NO: 25 - protein, 27 aa, synthetic construct
  MOD_RES 27: note = Amidated Proline
ISAFLGIPFAEPPMGPRRFLPPEPKKP

SEQ ID NO: 26 - protein, 9 aa, synthetic construct
  MOD_RES 9: note = Amidated Proline
GCRERDGSP

SEQ ID NO: 27 - protein, 5 aa, synthetic construct
LPRTG

SEQ ID NO: 28 - protein, 11 aa, synthetic construct
GCREGGIKVAV

SEQ ID NO: 29 - protein, 16 aa, synthetic construct
  MOD_RES 1: note = Acetylated glycine
  MOD_RES 16: note = Amidated glycine
GCRDVPMSMRGGDRCG

SEQ ID NO: 30 - protein, 21 aa, synthetic construct
  MOD_RES 1: note = Acetylated glycine
  MOD_RES 21: note = Amidated glycine
GCRDLPRTGGPQGIAGQDRCG

SEQ ID NO: 31 - protein, 13 aa, synthetic construct
  MOD_RES 1: note = Acetylated glycine
  MOD_RES 13: note = Amidated glycine
GCRDLPRTGDRCG

SEQ ID NO: 32 - protein, 24 aa, synthetic construct
  MOD_RES 1: note = Acetylated Proline
  MOD_RES 16: note = Amidated glycine
  MOD_RES 24: note = Amidated tyrosine
PHSRNGGGKGGGERCGGGRGDSPY

SEQ ID NO: 33 - protein, 47 aa, synthetic construct
  MOD_RES 1: note = Acetylated glycine
  MOD_RES 47: note = Amidated Cysteine
GGYGGGPGGPPGPPGPPGPPGPPGFOGDRGPPGPPGPPGPPGPPGPC

SEQ ID NO: 34 - protein, 20 aa, synthetic construct
  MOD_RES 1: note = Acetylated glycine
  MOD_RES 20: note = Amidated Proline
GCREGGGGGGGGGGGRGDSP

SEQ ID NO: 35 - protein, 35 aa, synthetic construct
  MOD_RES 1: note = Acetylated cysteine
  MOD_RES 35: note = Amidated Proline
CGGPOGPOGPOGPOGPOGPOGPOGPOGPOGRGDSP

SEQ ID NO: 36 - protein, 24 aa, synthetic construct
  MOD_RES 1: note = Acetylated proline
  MOD_RES 16: note = Acetylated glycine
  MOD_RES 24: note = Amidated tyrosine
PHSRNGGGKGGGERCGGGRGDSPY

SEQ ID NO: 37 - protein, 47 aa, synthetic construct; identical to SEQ ID NO: 11
  MOD_RES 1: note = Acetylated glycine
  MOD_RES 47: note = Amidated cysteine

SEQ ID NO: 38-40 - skipped (3 entries)

SEQ ID NO: 41 - protein, 18 aa, synthetic construct
  MOD_RES 1: note = Acetylated Lysine
  MOD_RES 18: note = Amidated valine
KKGCREPLQPVYEYMVGV

SEQ ID NO: 42 - protein, 21 aa, synthetic construct
  MOD_RES 1: note = Acetylated glycine
  MOD_RES 21: note = Amidated glycine
GCRDLPRTGGPQGIWGQDRCG

SEQ ID NO: 43 - protein, 8 aa, synthetic construct
GPQGIWGQ

SEQ ID NO: 44 - protein, 9 aa, synthetic construct
GCREYIGSR

SEQ ID NO: 45 - protein, 5 aa, synthetic construct
YIGSR

(19) United States
(12) Patent Application Publication
(10) Pub. No.: US 2026/0242723 A1
(43) Pub. Date: Aug. 20, 2026

(54) HYBRID HYDROGELS FOR CULTURING ENDOMETRIAL AND STROMAL ORGANOID CELLS

(71) Applicant: Massachusetts Institute of Technology, Cambridge, MA (US)

(72) Inventors: Samantha HOLT, Cambridge, MA (US); Linda G. GRIFFITH, Cambridge, MA (US); Lauren Jae PRUETT, Cambridge, MA (US)

(21) Appl. No.: 19/650,926

(22) Filed: Apr. 17, 2026

Related U.S. Application Data
(63) Continuation-in-part of application No. PCT/US2023/077081, filed on Oct. 17, 2023.
(60) Provisional application No. 63/892,868, filed on Oct. 3, 2025.

(51) Int. Cl. C12N 5/00; C08L 5/08; C12N 5/071; C12N 5/077

(52) U.S. Cl. CPC C12N 5/0068; C08L 5/08; C12N 5/0062; C12N 5/0661; C12N 5/069; C12N 2513/00; C12N 2533/20; C12N 2533/50; C12N 2533/54; C12N 2533/80; C12N 2537/10

(57) ABSTRACT

Hydrogels for organogenesis support organogenesis from mammalian cells, including human cells. The hydrogels contain a branched polyalkylene glycol functionalized with adhesion ligand peptides, such as integrin peptides and ECM peptides. In some forms, the hybrid hydrogels include a network of crosslinked branched synthetic polymers including a branched polyalkylene oxide polymer and a thiolated extracellular matrix polymer, preferably hyaluronic acid. In some forms, the synthetic hydrogels contain a branched polyalkylene glycol functionalized with GFOGER, RGD, YIGSR; and one or more ECM peptides, such as peptide(s) having affinity to fibronectin and/or peptide(s) having affinity to collagen and laminin. In some forms, the synthetic hydrogels possess desirable properties (e.g., a swelling ratio of ≤1.5 and/or a stiffness of ≤150 Pa) that allow the formation of perfusable vascular models in microfluidic devices. Also provided are methods of making the hydrogels and methods of using the hydrogels for organogenesis.

### CROSS-REFERENCE TO RELATED APPLICATIONS

This application is a continuation-in-part of PCT/US2023/077081 filed under the Patent Cooperation Treaty on Oct. 17, 2023, and claims the benefit of and priority to U.S. Provisional Application No. 63/892,868 filed Oct. 3, 2025, which are herein incorporated by reference in their entireties.

### STATEMENT REGARDING FEDERALLY SPONSORED RESEARCH

This invention was made with government support under EB029132 and HD110335 awarded by the National Institutes of Health. The government has certain rights in the invention.

### REFERENCE TO SEQUENCE LISTING

The Sequence Listing submitted as an XML file named “MIT_25192_CIP_US_ST26.xml” created on Apr. 17, 2026, and having a size of 55,510 bytes is hereby incorporated by reference pursuant to 37 C.F.R. § 1.834(c)(1).

### FIELD OF THE INVENTION

The invention is generally in the field of synthetic hydrogels and methods of making and use thereof, more specifically synthetic hydrogels for culture of endometrial cells, especially endometrial epithelial cells, stromal cell organoids, and vascular cells.

### BACKGROUND OF THE INVENTION

Organoids are three-dimensional structures derived from stem cells with organ-specific cell types and microarchitecture similar to the tissue of origin. These models offer a closer look into organogenesis unique to human that are not possible with animal models. In addition, human organoids allow for personalized medicine and autologous transplants using patient-derived crypts or stem cells cultured and expanded in vitro. However, stem cell-derived organoids suffer several limitations, primary of which is the reliance on natural hydrogels as a 3D scaffold, such as MATRIGEL®.

MATRIGEL® is a commercial product widely used to provide the 3D scaffold for the growth of organoids of all cell types. However, the reliance on MATRIGEL®, or similar naturally derived biopolymer matrices, as the scaffold for organoid growth introduces a number of significant limitations into the study and use of the resultant organoids, and severely limits further development of the field. MATRIGEL® is derived from a basement membrane extracellular matrix (ECM)-rich mouse sarcoma and therefore introduces a significant risk of immunogen or pathogen transfer if given to a patient. Additionally, the batch-to-batch variability of MATRIGEL® may lead to inconsistent cell behaviors, introducing unknown and potentially confounding variables that complicate the interpretation of both basic and translational research. Further, the role of MATRIGEL® in organoid formation remains poorly understood. ECM is known to implicate in the regulation of tissue development and function. The specific roles of ECM factors are difficult to ascertain using MATRIGEL® given that its molecular components cannot be readily manipulated. It is unclear whether MATRIGEL® serves merely as a passive 3D scaffold providing physical support to the growing organoid, or actively influences organoid formation by providing essential biological cues. The cumbersome process to remove MATRIGEL® and recover the organoids for downstream analysis is an additional challenge.

Additionally, organoids derived from induced pluripotent stem cells (iPSC) result in complex tissue structures (for example, iPSC-derived intestinal organoids typically have smooth muscle and stroma surrounding epithelial cells (McCauley et al., Dev. 144:958-962 (2017)), but cell maturation is limited to fetal stages. There is great interest in organoids derived from postnatal tissue biopsies, as these capture features of (patho)-physiological states of mature tissues, such as epigenetic changes and somatic mutations associated with donor tissue's natural or diseased state.

PCT/US2020/044067 entitled “Synthetic Hydrogels for Organogenesis” filed by Massachusetts Institute of Technology on Jul. 29, 2020, describes a synthetic gel performing as well as MATRIGEL but with more consistency and no potentially tumorigenic ingredients. The gel is formed of a branched synthetic polymer, typically an 8 kD polyethylene glycol, extracellular matrix binding (ECM) peptides that are functionalized to crosslink the PEG, and peptides comprising adhesion. The adhesion ligands are typically cell-binding peptides, for example, integrin binding peptides. The integrin binding peptides can be derived from collagen, fibronectin, and/or laminin. Preferred gels include the amino acid sequence GFOGER (SEQ ID NO:1). The ECM binders are typically synthetic peptides with affinity to extracellular matrix components, preferably the ECM polymers. For example, an ECM binder can be a peptide having affinity to fibronectin, collagen, laminin, other ECM components, or combinations thereof.

Microvascular morphogenesis protocols rely heavily on fibrin, which serves as a provisional wound-healing ECM in vivo. As the expectations for reproducibility and biological performance of MPS continue to rise, the short-comings of fibrin—including lot-to-lot variability, undesirable biochemical cues, limited modularity, and poorly-controlled degradation⁹⁻¹¹—are becoming increasingly apparent. These shortcomings are accentuated in applications that incorporate additional cell types to create functional tissue mimics, as fibrin may impair or otherwise not support biological functions. For example, human mucosal epithelia, such as gut and endometrium, are expanded and cultured as organoids in basement membrane-mimicking Matrigel, which shares many of fibrin's performance limitations but offers a complex mix of biological cues that support epithelia¹²⁻¹⁴. While a combination of fibrin and Matrigel facilitates a limited vascularization of gut organoids¹⁵, a more reproducible and biologically versatile ECM for creating complex microvascularized tissues is desirable for MPS translation into the drug development pipeline. However, no completely synthetic extracellular matrix (CS-ECM) for vessel formation by morphogenesis in vitro has yet been reported³⁻⁵.

There remains a need for fully-defined hydrogels supporting organogenesis from biopsied human endometrial cells and other cells with more complex responses to their environment, such as neural cells, with control over complex cell-matrix interactions.

There is also a need for compositions for producing perfusable vascular models in vitro.

Therefore, it is an object of the present invention to provide hybrid hydrogels for organogenesis from endometrial and stromal cells and organoids.

It is another object of the present invention to provide methods of making and using the hybrid hydrogels.

It is a further object of the present invention to provide compositions for culturing endothelial cells.

It is a further object of the present invention to provide compositions for culturing endothelial cells in microfluidic devices.

It is a further object of the present invention to provide methods of producing vascular models in vitro.

It is a further object of the present invention to provide methods of producing vascular models in microfluidic devices.

### SUMMARY OF THE INVENTION

A polymeric hydrogel material formulation (also referred to as a “synthetic hydrogel” or “hydrogel”) has been developed for use as a three-dimensional (3D) culture substrate in cell and organoid culture.

The synthetic hydrogel generally includes a branched polyalkylene glycol, such as a branched polyethylene glycol, functionalized with adhesion ligand peptides. The adhesion ligand peptides can allow for binding of cell surface receptors as well as cell-secreted proteins, such as integrin binding peptides and extracellular matrix (ECM) binding peptides.

Typically, the molar ratio of the one or more integrin peptides to the one or more ECM binding peptides is greater than 1:1.

In some forms, the integrin binding peptides forming the synthetic hydrogel can be peptides derived from collagen, fibronectin, and/or laminin. For example, the integrin binding peptides forming the synthetic hydrogel are or include GFOGER (SEQ ID NO:1), RGD (SEQ ID NO:3), and YIGSR (SEQ ID NO:45).

In some forms, the ECM binding peptides forming the synthetic hydrogel are peptide(s) having affinity to fibronectin (or “FN binder”) and/or peptide(s) having affinity to collagen and laminin (or “BM binder”). For example, the ECM binding peptides forming the synthetic hydrogel are or include NH₂-GCREISAFLGIPFAEPPMGPRRFLPPEPKKP(Am) (SEQ ID NO:5) and NH₂-GCRE-TLQPVYEYMVGV-COOH (SEQ ID NO:6).

In some forms, the synthetic hydrogels also include a crosslinker, such as a chemically functionalized extracellular matrix polymer or a crosslinking peptide.

In some forms, the synthetic hydrogel is a hybrid hydrogel that includes a branched polyalkylene glycol, preferably PEG, functionalized with synthetic adhesion ligand peptides which allow the binding of cell surface receptors as well as cell-secreted proteins (such as integrin binding peptides and ECM binding peptides described above), and crosslinked with a chemically functionalized extracellular matrix polymer such as thiolated hyaluronic acid. A feature of this formulation is that the peptides are bound to the multi-arm PEG, not the hyaluronic acid. There is a plethora of gels present in the literature with adhesion peptides directly bound to hyaluronic acid, which limits the mobility of the peptides compared to peptides bound to PEG.

It has been found that this hydrogel containing chemically modified hyaluronic acid (HA), a glycosaminoglycan that is a significant component of the extracellular matrix (“ECM”) in mammalian tissues, instead of crosslinking peptides, alters the properties of the hydrogel and induces changes in the cells and organoids cultured therein.

Other ECM could be used together with the HA, or in place of the HA. The inclusion of HA changes the biological properties of the gel by supporting the binding of cell surface HA receptors and remodeling in response to HA-degrading hyaluronidase enzymes. The inclusion of HA also changes the physiochemical properties of the gel by modifying the ligand distribution (compared to a gel made from only PEG+crosslinking and adhesion ligand binding peptides), decreasing the swelling of the gel in aqueous media, which is important for inclusion in microfluidic platforms, and decreasing the total mass of polymer required to obtain the same ligand concentration and bulk mechanical properties. Due to the difference in physiochemical properties of PEG and HA, identifying compositions where the two polymers are interspersed and able to gel required significant testing. Inclusion of the bioactive peptide ligands on the arms of the PEG molecule increases their accessibility for binding by cell surface proteins, as opposed to alternative gel formulations which attach peptide ligands to a HA backbone. Another advantage of the inclusion of the HA is that it makes the matrix hyaluronidase-degradable but not MMP-degradable.

Hydrogel precursor compositions are also disclosed. Typically, the hydrogel precursor composition contains the branched polymer functionalized with the adhesion ligand peptides, such as one or more integrin peptides and one or more ECM peptides in a suitable carrier, such as a buffer. The hydrogel precursor composition can be combined with a crosslinker, such as a chemically functionalized extracellular matrix polymer or a crosslinking peptide, to initiate crosslinking and form the synthetic hydrogel. In some forms, the hydrogel precursor compositions also contain one or more desired types of cells, (e.g., endothelial cells optionally in combination with stromal cells, macrophages, and/or endometrial epithelial cells or organoids), such that the cells are encapsulated in the hydrogel following crosslinking.

In some forms, the synthetic hydrogels includes a branched polyalkylene glycol, preferably PEG, functionalized with synthetic adhesion ligand peptides which allow the binding of cell surface receptors as well as cell-secreted proteins (such as integrin binding peptides and ECM binding peptides described above), and crosslinked with a crosslinking peptide. In some forms, synthetic hydrogels including these components are suitable for vascularized organogenesis. In some forms, the vascularized organogenesis is formed in a microfluidic device. In some forms, the synthetic hydrogels exhibit properties that are particularly suitable for desirable for forming vasculature in microfluidic devices. For example, the synthetic hydrogels have a swelling ratio of ≤1.5 and a stiffness of ≤150 Pa.

In some forms, the synthetic hydrogels are useful for supporting vascular morphogenesis in vitro. In some forms, the synthetic hydrogel formation occurs in a microfluidic device, and the subsequent vascular tissue formation occurs in the microfluidic device with a continuous flow culture media at a suitable flow rate and a suitable pressure drop across the channel of the microfluidic device. Different cell types can be used in the synthetic hydrogel to form various vascular models, such as vasculature with open lumen or vascular lesions.

### BRIEF DESCRIPTION OF THE DRAWINGS

FIG. 1 is a schematic overview of making the crosslinked HA adhesive gel.

FIG. 2A is a graph of the Gel shear storage modulus (G′) (Pa) and shear loss modulus (Pa), showing that it increases with increasing concentrations of hyaluronic acid (HA). FIGS. 2B-2E are micrographs of the cells in culture, showing morphology as a function of HA concentration.

FIGS. 3A-3E show that epithelial organoids exhibit similar morphology in soft gels, but different morphology in stiff gels. FIG. 3A is a graph of the shear storage moduli of gel formulations used for EEO culture (PEG-peptide, PEG-HA (0.25%), stiff PEG-peptide, and stiff PEG-HA (0.38% HA-SH). FIGS. 3B-3E is the morphology of patient-derived human endometrial epithelial cells (“EEOs”) in 3D in soft PEG-peptide (FIG. 3B), stiff PEG-peptide (FIG. 3C), soft PEG-peptide-HA (FIG. 3D), and stiff PEG-peptide-HA hydrogels (FIG. 3E). Scale bar=200 um. References: (1) Edelstam G A B. J Histochem Cytochem. 39(8):1131-1135 (1991). (2) Fouladi-Nashta A A. Reproduction. 2017; 153(2):R43-R58. (3) Harden S L. Front Cell Dev Biol. 9:626619 (2021). (4) Hernandez-Gordillo V. Biomater. 254:120125 (2020).

FIGS. 4A-4B is a comparison of hybrid hydrogel formulations made with varied concentrations and molecular weights of hyaluronic acid (HA), including the volumetric swelling factor (FIG. 4A) and shear storage modulus (FIG. 4B).

FIGS. 5A-5C are fluorescence micrograph images of primary human adipocytes encapsulated in three different hydrogel formulations: commercially available HYSTEM-C (FIG. 5A), a peptide-crosslinked PEG hydrogel containing cell- and ECM-adhesive binder peptides based on PCT/US2020/044067 (FIG. 5B), and the hybrid PEG-HA hydrogel (FIG. 5C). The cells are stained with a LIVE/DEAD viability stain (Invitrogen).

FIGS. 6A-6E are representative fluorescence micrograph images from a pilot study comparing the performance of three different hydrogel formulations in a nerve-on-a-chip model using spheroids of human induced pluripotent stem cell-derived neurons. The samples were stained for beta-3-tubulin (green), a marker of neural phenotype, and f-actin (red), a marker of non-neuronal cells. The images depict commercially available MATRIGEL (FIG. 6A), a peptide-crosslinked PEG hydrogel containing cell- and ECM-adhesive binder peptides based on PCT/US2020/044067 (FIGS. 6B, 6C), and the hybrid PEG-HA hydrogel (FIGS. 6D,6E) used for culture of the spheroids over the course of 37 days.

FIGS. 7A-7C show an overview of synthetic hydrogel for perfusable microvasculature. FIG. 7A is a schematic showing the v-CS-ECM composed of an 8-arm PEG-VS functionalized with integrin-binding and cell matrix-binding peptides and crosslinked with an MMP-degradable peptide for cell-mediated remodeling. FIG. 7B is a schematic showing the process of mixing v-CS-ECM with endothelial cells and supporting cells prior to injection in a microfluidic device and the vascular self-assembly over the course of a week. FIG. 7C is a schematic showing that hydrogel, cellular, device, and tissue bed parameters were varied to determine the impact on network formation in the v-CS-ECM.

FIGS. 8A-8D show the results of the hydrogel and cell parameter static endothelial network screen. FIG. 8A shows that using the upper end of concentrations reported for fibrin (12M HUVECs/mL) a softer hydrogel (3.0 wt % 0.52×link) compared to the previously published organoid formulation (3.6 0.43×link) results in greater endothelial network area fractions which is further enhanced with the addition of YIGSR. FIG. 8B shows that using the v-CS-ECM (3.0 wt % 0.52×link+YIGSR), 18M HUVECs/mL resulted in significantly greater network area coverage compared to 12M and 15M HUVECs/mL. FIG. 8C shows 18M HUVECs/mL a 7:1 ratio of HUVECs to lung fibroblasts results in the greatest network area coverage. FIG. 8D shows that using 18M HUVECs/mL and 7:1 HUVEC:NHLF the difference in network formation between gel formulations is more pronounced, with the v-CS-ECM creating a significantly greater network area fraction compared to the same stiffness hydrogel without YIGSR (3.0 wt % 0.52×link) and the published organoid formulation (3.6 0.43×link). Scale Bar: 200 um. Each data point represents a device. Max intensity projections from Day 5 shown. Data represented as mean±standard deviation. Statistics: ANOVA with post-hoc tests. *p<0.05, **p<0.01, ***p<0.001, ****p<0.0001.

FIGS. 9A-9D show a transition from static to continuous flow culture results in perfusable microvasculature. FIG. 9A shows the change in device geometry once the static culture conditions transitioned to continuous flow driven by peristaltic pumps. The device geometry was adapted to have a simple looped flow or serpentine looped flow that had a measured pressure difference of 25 mm H₂O across the channel. FIG. 9B is an image of a full device view of networks cultured in static conditions that shows an interconnected network without perfusability observed. FIG. 9C is an image of a full device view of networks cultured in a looped device without a pressure differential that demonstrated perfusability at the end of the channel evidenced by beads trafficking through networks (zoomed in bead only view in the white boxed area). FIG. 9D is an image of a full device view of networks cultured in a serpentine device with a pressure differential that demonstrated perfusability across the channel evidenced by beads trafficking through networks (zoomed in bead only view in the white boxed area). Day 7 images. Scale Bar: 500 μm.

FIGS. 10A-10D show vessel formation in continuous flow conditions. FIG. 10A is an image showing that self-assembly of HUVEC-NHLF networks over 1-week results in perfusable vessels in a device with continuous flow and a pressure differential. FIG. 10B is a bar graph showing that diameter does not significantly change between static and pumped conditions. FIG. 10C is a bar graph showing network complexity assessed by the number of branch points is significantly reduced in flow conditions. FIG. 10D is a plot showing that pumped cultures have significantly greater MMP-2 secreted in spent medium compared to static culture conditions, corresponding with greater perfusable network formation. Each data point represents a device. Scale Bar: 500 μm. Data represented as mean±standard deviation. **p<0.01.

FIGS. 11A-11E show incorporation of macrophages in HUVEC-NHLF networks. FIG. 11A is Day 5 confocal images of HUVEC-NHLF-macrophage networks in static culture with varying ratios of fibroblasts to macrophages (macrophages in red labeled with CellTracker). FIG. 11B is a bar graph showing that as the macrophage ratio increases, the area fraction and diameter of the endothelial networks decreases. FIG. 11C is a plot showing that macrophages are functional in culture and secrete MMP-9. FIG. 11D is a plot showing MMP-2 secretion over 7-day static culture in HUVEC-NHLF-macrophage culture. FIG. 11E is a series of images showing that transitioning the 6:1 NHLF to macrophage ratio to pumped culture, the HUVEC-NHLF-macrophage networks are perfusable as evidenced by bead trafficking through the endothelial networks (green). Scale Bar: 200 μm. Statistics: ANOVA, Tukey post-hoc test. *p<0.05, **p<0.01, ***p<0.001, ****p<0.0001. Data represented as mean±standard deviation. N=3 devices.

FIGS. 12A-12G show uterine endothelial networks. FIG. 12A is a series of Day 5 images of GFP-labeled uterine endothelial cell (HUTMVEC)—endometrial stromal cell (ESC) networks in static conditions. FIG. 12B and FIG. 12C are bar graphs showing that the same concentration of endothelial cells and ratio of endothelial cells to fibroblasts (18M 7:1) resulted in the greatest network area coverage and network diameter. FIG. 12D is a series of images showing HUTMVEC-ESC networks were exposed to continuous flow in a serpentine-loop device and self-assembled into perfusable vessels over a course of 7 days as seen by red beads trafficking through the networks (labeled with GFP) at Day 7. FIG. 12E is a bar graph showing that in pumped conditions the vessel diameter was significantly greater compared to static, FIG. 12F shows there was not a significant difference in number of branch points representing network complexity, and FIG. 12G shows more MMP-2 was detected over time in spent culture media. Each data point represents a device, N=3-6 devices. Scale Bar: 200 μm. *p<0.05, **p<0.01, ***p<0.001, ****p<0.0001. Data represented as mean±standard deviation.

FIGS. 13A-13E show incorporation of organoids in uterine microvascular networks. FIG. 13A is a schematic that shows endometriosis lesions establish complex microenvironments of epithelial, stromal, endothelial, and immune cells. FIG. 13B is a schematic showing that droplet hydrogel cultures were used to assess organoid emergence from single cell. FIG. 13C is a series of images showing V-CS-ECM supports organoid emergence from single cell over the course of a week. FIG. 13D is a schematic showing how EEOs, HUTMVECs, and ESCs were combined in a hydrogel precursor prior to injection in a microfluidic device and cultured for a week. FIG. 13E is a series of images showing V-CS-ECM supports EEO growth within HUTMVEC-ESC networks. Scale Bar: 500 μm.

FIGS. 14A-14C show CAD snapshots of custom microfluidic devices. FIGS. 14A and 14B show the dimensions of the static device used in this study. FIG. 14C shows the dimensions of the serpentine channel added to the device to facilitate flow studies with a continuous pressure drop. The tissue channel geometry is the same as shown in FIGS. 14A-14B.

FIG. 15A is a plot showing in situ gelation curve for the 3.0 wt % 0.52 crosslinking hydrogel with YIGSR shows gelation is fully complete after 30 minutes at 37° C. FIG. 15B is a bar graph showing the swelling ratio for the three hydrogel formulations tested in this study. The 3.0 wt % 52% crosslinking hydrogel formulations swelled ~25% less compared to the original organoid formulation published in Gnecco et al²⁰. FIG. 15C is a bar graph showing bulk stiffness measurements using rheology to compare the three gel formulations. The vascular formulation was ~150 Pa storage modulus and 25% softer.

FIG. 16 is a series of images to monitor the daily network progression in each of the different gel formulations used in this study cultured in static conditions. Concentration of HUVECs: 18M/mL and 7:1 HUVEC:NHLF ratio.

FIG. 17A is a bar graph showing that serpentine devices were measured to have a 25 mm pressure drop across the channel. Each data point represents a different device and pump setup tested. FIG. 17B is an image of the pumped experiment setup. PDMS devices were bonded to a glass slide and placed into a 3D printed holder connected to tubing and a peristaltic pump with the same footprint as a well plate to allow for easy imaging. FIG. 17C is a list and image of components involved in the tubing setup and information for sterilization.

FIGS. 18A and 18B are COMSOL simulations of (FIG. 18A) fluid shear stress on hydrogel and (FIG. 18B) pressure in the 4-post simple-loop device used in this study. FIGS. 18C and 18D are COMSOL simulations of (FIG. 18C) fluid shear stress on hydrogel and (FIG. 18D) pressure in the 4-post serpentine-loop device used in this study.

FIG. 19A is a bar graph showing quantification of perfusable vessels throughout the entire device. Quantification of perfusability was performed at experiment end point between 7 and 10 days for 10 devices. FIG. 19B is a schematic showing each section of the device, with all devices exhibiting perfusability in the bottom portion of the device and most devices having perfusable vessels in the top and bottom of the device.

FIG. 20 is a series of images showing vessels maintain stability for at least 14 days cultured under continuous flow.

FIG. 21 is a series of images showing attempts to decrease the concentration of endothelial cells with 15M HUVECs/mL and 7:1 HUVEC:NHLF ratio tested. While not as successful as 18M HUVECs/mL, perfusability was observed in the bottom quarter of the device.

FIGS. 22A-22C show second macrophage donor in HUVEC-NHLF-macrophage networks. FIG. 22A is a series of static confocal images taken on Day 5 of HUVEC-NHLF-macrophage networks formed in static culture with varying ratios of macrophages to lung fibroblasts with a different macrophage donor. FIG. 22B is a set of bar graphs showing that increasing the number of macrophages decreases area fraction and mean endothelial network segment diameter at day 5. FIG. 22C is a plot showing increasing MMP-9 secretion with increasing number of macrophages, demonstrating functionality. Data represented as mean±standard deviation. N=3.

FIGS. 23A and 23B show pumped HUVEC-NHLF-macrophage networks. FIG. 23A is a series of time course images of endothelial networks with a 6:1 nhlf to macrophage ratio in continuous pumped culture in serpentine devices. FIG. 23B is a plot showing that HUVEC-NHLF-macrophage networks similarly demonstrate an increase in MMP-2 detected in the supernatant compared to static culture. Data is represented as mean±standard deviation. N=3.

FIG. 24 is a series of images and bar graphs showing HUTMVEC-ESC networks form similarly with two different endometrial stromal cell donors with no significant difference in area fraction or diameter in static culture. Data is represented as mean±standard deviation. N=3.

FIGS. 25A-25D show experimentation of combination media. FIG. 25A is a table showing the composition of combination media. FIG. 25B is a schematic showing the HUTMVEC-ESC culture experimentation in a combination media (50% microvascular endothelial media, 50% endometrial organoid media without A8301 and Y27632) to form microvascular networks in static culture. FIG. 25C is a series of images showing networks formed to a similar extent in the combination media. FIG. 25D is a bar graph showing there is no significant difference in network diameters. Scale Bar: 200 μm.

FIG. 26 shows the E2 Presoak test. For a final experiment to combine endometrial epithelial organoids and vessels, it was necessary to use an estradiol containing medium to support organoid growth. As PDMS rapidly absorbs lipophilic compounds, it was necessary to develop a method to pre-saturate the PDMS to maintain a concentration of 10 nM E2 throughout the duration of the experiment. Presoaking the devices for 24 hours in a 10 nM E2 containing solution, prior to loading the gel maintained the concentration throughout the experiment by using media containing 10 nM E2. If devices were not presoaked, the media only contained approximately 50% of the desired concentration by day 6. N=2 devices per condition.

### DETAILED DESCRIPTION OF THE INVENTION

Synthetic polymeric hydrogels are a fundamental tool in tissue engineering, offering increased user tunability and reproducibility over natural polymer hydrogels. However, the development of a hybrid hydrogel which can support the co-culture of multiple different primary human cell types, especially hormonal responsive cell types such as endometrial epithelial cells and organoids thereof, remains challenging.

Synthetic hydrogels containing a branched polyalkylene glycol functionalized with synthetic adhesion ligand peptides, which allow the binding of cell surface receptors as well as cell-secreted proteins (such as integrin binding peptides and ECM binding peptides described above), and crosslinked with a crosslinking peptide, were developed. In some forms, the synthetic hydrogels exhibit properties that are particularly suitable for desirable for forming vasculature in microfluidic devices. For example, the synthetic hydrogels have a swelling ratio of ≤1.5 and a stiffness of ≤150 Pa.

Hybrid hydrogels, containing both naturally derived and synthetic polymers with both containing a substantial portion of the hydrated polymer mesh structure (at least 5% either naturally derived polymers or synthetic polymers by mass), were also developed using a combination of bioactive peptide-functionalized multi-arm poly(ethylene glycol) (PEG) and chemically modified HA. Hyaluronic acid (HA) is a naturally occurring linear polysaccharide which can be produced industrially and is chemically modifiable and endogenously bioactive. Additionally, HA is a component of the human endometrial extracellular matrix and its metabolism by endometrial stromal cells and perivascular cells is altered in decidualized cells in vitro. This hybrid gel has demonstrated effective use as a 3D culture substrate for primary human cells, including endometrial stromal and epithelial cells and adipocytes, as well as model peripheral neurons derived from human induced pluripotent stem cells.

### I. Definitions

As used herein, the term “hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer forms a gel which is not toxic to living cells and allows sufficient diffusion of oxygen and nutrients to the entrapped cells to maintain viability.

As used herein, the term “synthetic”, in the context of polymer, refers to a polymer that is produced solely through artificial chemical synthesis from organic precursor molecules. That is, the molecule is not produced by the metabolism of any living organism. A hydrogel is considered as a “synthetic hydrogel” as long as it contains a synthetic polymer. For example, hydrogel containing a synthetic polymer and natural polymer(s) and/or natural peptide(s) is considered a synthetic hydrogel.

As used herein, the term “natural”, in the context of polymer, refers to a polymer that can be produced by the metabolism of a living organism. Natural polymers can be derived from plant or animal tissue or can be produced via fermentation or recombinant synthesis by microorganisms.

As used herein, the term “hybrid”, in the context of hydrogels, refers to a hydrogel comprised of a combination of synthetic and natural polymers in a mass: mass ratio of at least 5% natural to synthetic or vice versa. A synthetic hydrogel is also considered a hybrid hydrogel if it contains a combination of synthetic and natural polymers in a mass ratio described above.

As used herein, the term “biodegradable”, in the context of polymer, refers to a polymer that will degrade or erode by enzymatic action and/or hydrolysis under physiologic conditions to smaller units or chemical species that are capable of being metabolized and/or eliminated.

As used herein, the terms “binder”, “adhesion ligand”, “adhesion ligand binding peptide”, “adhesion ligand binder”, “adhesion binder”, “adhesion peptide” and “peptide binder” are used interchangeably and refer to peptides, preferably synthetic peptides, with affinity to one or more proteins, typically to cell adhesion molecules (CAMs). The proteins may be cell-associated proteins and/or cell-secreted proteins. Exemplary cell adhesion molecules include integrins, immunoglobulin superfamily (IgSF) CAMS, cadherins, and selectins. The adhesion ligands may derive from proteins of the ECM. The adhesion ligands may be linear or branched.

As used herein, the terms “inhibitor”, “apoptosis inhibitor” refers to small molecules with known inhibitory effects on cellular apoptosis. The inhibitor may inhibit any stage of cellular apoptosis. Preferably the inhibitor inhibits dissociation-induced apoptosis. The reduction in apoptosis in a cell population may be compared to a control. The control may include the same type, or similar cells, grown under the same conditions, in the absence of the inhibitor.

As used herein, the term “molecular weight”, in the context of polymers, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, the term “molecular weight”, in the context of biological molecules, such as peptides, proteins, glycoproteins, etc., generally refers to the molar mass of the molecule in g/mol, or Daltons, and may be measured by any suitable technique, such as electrophoresis, or mass spectrometry.

As used herein, the term “functionalize” refers to modifying in a manner that results in the attachment of a functional group or moiety. For example, a molecule may be functionalized by the introduction of a functional group which makes the molecule a strong nucleophile or strong electrophile. For example, a molecule, such as hyaluronic acid, may be functionalized to introduce a thiol, amine, acrylate, or quinone.

As used herein, the term “peptide” refers to a biological molecule, which may include “polypeptide,” and/or “oligopeptide,” and refers to a chain of natural and/or synthetic amino acid residues linked together by covalent bonds (e.g., peptide bonds). The length of the peptide is limited at the lower end only by the minimum number amino acids required to form a self-assembling peptide.

As used herein, the term “extracellular matrix”, “ECM” refers to the components and/or the network of extracellular macromolecules, such as proteins, enzymes, and glycoproteins, that provide structural and biochemical support of surrounding cells. The extracellular matrix includes the interstitial matrix and the basement membrane components of the ECM include proteoglycans heparan sulfate, chondroitin sulfate, keratan sulfate; non-proteoglycan polysaccharide hyaluronic acid, and proteins collagen, elastin, fibronectin, and laminin.

As used herein, the term “extracellular matrix binding peptide” refers to a synthetic peptide with affinity to ECM components.

As used herein, the term “hydrogel matrix” typically refers to the network of cross-linked polymers forming the hydrogel. The hydrogel matrix may or may not include the binders.

As used herein, the term “crosslinker” refers to a molecule containing two or more reactive moieties which are used to connect two or more polymer molecules via chemical bonds into a larger 3D mesh structure characteristic of a hydrogel. The bonds may be in the form of covalent bonds or ionic bonds.

As used herein, the term “reactive functional moiety” refers to a moiety containing any known suitable chemical functional group that reacts with another chemical functional group in the given context (i.e., reaction conditions) to form a bond, as exemplified herein. The bond may be in the form of covalent bonds or ionic bonds.

As used herein, the term “amino acid” refers to standard nomenclature, amino acid residue as denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “cleavable motif”, in the context of proteins, refers to a sequence of amino acids, such as to a sequence of two, three, four, five, six, etc., amino acids that are recognized by one or more proteolytic enzymes, such as proteases, proteinases, peptidases, and transpeptidases, such as protein sorting transpeptidases. The cleavable motif may be a synthetic or a naturally occurring recognition site for the proteolytic enzymes.

“Mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. For example, the cell may be a stem cell. Immortalized cells are also included within this definition. In some embodiments, the cell has been genetically engineered to express a recombinant protein and/or nucleic acid.

“Autologous” refers to a transplanted biological substance taken from the same individual.

“Allogeneic” refers to a transplanted biological substance derived from a different individual of the same species.

“Xenogeneic” refers to a transplanted biological substance taken from a different species.

As used herein, the term “variant” refers to a peptide or a polypeptide that differs from a reference peptide or a polypeptide but retains the same mechanism of activity. A typical variant of a peptide or a polypeptide differs in amino acid sequence from another, reference peptide or a polypeptide. Generally, differences are limited so that the sequences of the reference peptide or a polypeptide and the variant are closely similar overall and, in many regions, identical. A variant and reference peptide or a polypeptide may differ in amino acid sequence by one or more modifications (e.g., substitutions, additions, and/or deletions). A substituted or inserted amino acid residue may or may not be one encoded by the genetic code. A variant of a peptide or a polypeptide may be naturally occurring such as an allelic variant, or it may be a variant that is not known to occur naturally.

Modifications and changes can be made in the structure of the peptide or a polypeptide of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, and antigens. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). In particular, embodiments of the polypeptides can include variants having about 50%, 60%, 70%, 80%, 90%, and 95% sequence identity to the polypeptide of interest.

Use of the term “about” is intended to describe values either above or below the stated value in a range of approx. +/−10%; in other embodiments the values may range in value either above or below the stated value in a range of approx. +/−5%.

### II. Compositions

### A. Synthetic Hydrogels

The disclosed synthetic hydrogels contain: (1) a synthetic polymer (e.g., a branched polyalkylene glycol (“PAG”), such as a 4 arm or 8 arm polyethylene (glycol) (“PEG”)), and (2) one or more adhesion ligand binding peptides (also referred to herein as “adhesion peptides”, “adhesion binders”, “adhesion ligands”, “adhesion ligand binders”, “peptide binders”, or “binders”). Optionally, the synthetic hydrogels further contain one or more crosslinkers, such as ECM polymers, small molecules, or peptides. Preferably, the synthetic hydrogel contains two or more adhesion peptides, such as one or more integrin peptides and one or more extracellular matrix (ECM) binding peptides. The terms “synthetic hydrogel,” “hydrogel matrix”, and “synthetic hydrogel cell culture matrix” are used interchangeably herein.

The synthetic hydrogels can be tailored to the micro-environmental properties and components of specific tissues or particular biological states, such as varying phases of developmental, points in time dependent physiological processes such as reproductive cycles, or disease states. These can be used to create fully defined hydrogel matrix for the expansion of cells and organoids with a hydrogel of precise biomechanical and biological properties. These synthetic hydrogels also offer a window of opportunity for large-scale production of clinical-grade cells and tissues, especially human cells and tissues. The biomechanical and biological properties of the synthetic hydrogels can be “tuned” by varying polymer structures, polymer molecular weight, and binders to support organoid growth from different types of cells, normal or tumor-derived. For example, hybrid hydrogels formed from multi-arm polyethylene glycol (PEG), integrin binding peptides, and ECM binding peptides support human endothelial cell growth to form various vasculature tissues.

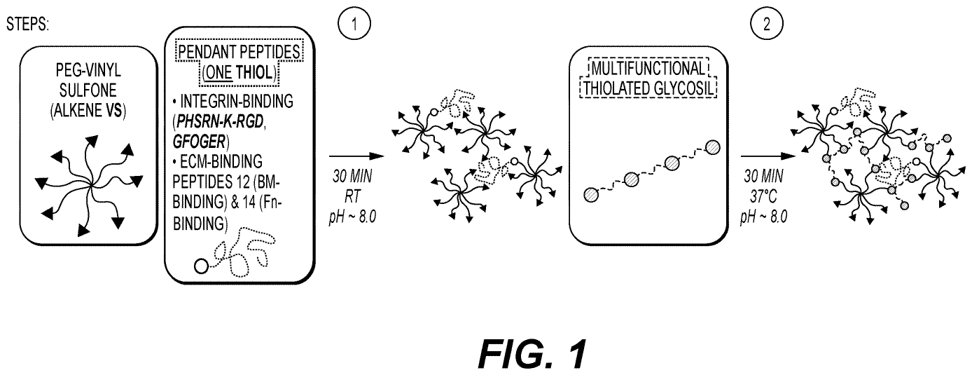

In some forms, as illustrated in FIG. 1, the hydrogels are hybrid hydrogels having three main components: (1) a branched polyalkylene glycol such as an 8 arm polyethylene (glycol) (“PEG”), (2) peptide binders bound to the PEG to form a polyalkylene glycol-peptide adduct, and (3) thiolated ECM polymer such as hyaluronic acid, but may be a different ECM polymer or in combination with a different ECM, crosslinking the polyalkylene glycol-peptide adduct.

The hybrid hydrogels formed from synthetic polymers with binders and ECM polymers provide chemically defined and versatile cell cultures as alternatives to natural, animal-derived matrices such as MATRIGEL®. The composition of these hybrid hydrogels can be tailored to the micro-environmental properties and components of specific tissues or particular biological states, such as varying phases of developmental, points in time-dependent physiological processes such as reproductive cycles, or disease states. These can be used to create fully defined hydrogel matrix for the expansion of cells and organoids with a hydrogel of precise biomechanical and biological properties. These hybrid hydrogels also offer a window of opportunity for large-scale production of clinical-grade cells and tissues, especially human cells and tissues. The biomechanical and biological properties of the hybrid hydrogels can be “tuned” or optimized by varying polymer structures, polymer molecular weight, and binders to support organoid growth from different types of cells, normal or tumor-derived. For example, hybrid hydrogels formed from multi-arm polyethylene glycol (PEG), integrin binding peptides, and approximately 300 kDa molecular weight hyaluronic acid (HA) support human endometrial epithelial cell growth.

### 1. Components

The components forming the synthetic hydrogel can be provided either separately or combined, in dry form, such as powders, or in solution form. The hydrogel forms when cross linking conditions are applied. For example, the components of the hydrogel are dissolved in a liquid, such as water or medium, and combined, either in a single step or multiple steps, to form a pre-gel composition, and the temperature is increased to induce cross linking of the pre-gel composition and form the synthetic hydrogel. Typically, the synthetic hydrogel contains the one or more integrin peptide and one or more ECM peptides at a molar ratio greater than 1:1. In some forms, the synthetic hydrogel is a hybrid hydrogel that further contains a natural polymer, such as an ECM polymer, e.g., hyaluronic acid.

i. Synthetic Polymers

The synthetic hydrogel contains one or more synthetic polymers. An exemplary synthetic polymer is polyalkylene glycol, such as polyethylene glycol (“PEG”).

Typically, the PEG has a molecular weight greater than 10 kDa, such as between about 10 kDa and 100 kDa. The molecular weight of the PEG may be between about 10 kDa and about 90 kDa, between about 10 kDa and about 80 kDa, between about 10 kDa and about 70 kDa, between about 10 kDa and about 60, such as about 50 kDa, about 40 kDa, about 30 kDa, or about 20 kDa. Preferably, the molecular weight of the PEG is about 20 kDa or about 40 kDa. Representative PEG molecular weights include 10 kDa, 15 kDa, 20 kDa, 30 kDa, 50 kDa, and 100 kDa. PEG of any given molecular weight may vary in other characteristics such as length, density, and branching. In some forms, the PEG is 8-arm with a MW of 20 kDa. In some forms, the PEG is 8-arm with a MW of 40 kDa. In some forms, PEGs of various molecular weights can be used, for example, a mixture of 20 kDa PEG and 40 kDa PEG.

When more than one PEG is used to form the synthetic hydrogel, the plurality of synthetic polymers may include PEGs of various molecular weights, for example, a mixture of 20 kDa PEG and 40 kDa PEG. The plurality of synthetic polymers may include PEGs of at least two molecular weights, a lower molecular weight PEG and a higher molecular weight PEG, where the ratio of the lower molecular weight PEG to a higher molecular weight PEG is about 1:0.1, about 1:0.25, about 1:0.5, about 1:1, about 1:1.5, about 1:2; about 1:2.5, about 1:3, or more. For example, in a hydrogel containing a 20 kDa 8-arm PEG and a 40 kDa 8-arm PEG, the ratio of the 20 kDa to 40 kDa PEG is about 1:0.1, about 1:0.25, about 1:0.5, about 1:1, about 1:1.5, about 1:2; about 1:2.5, about 1:3, or more, preferably about 1:1.

The synthetic polymer(s) is branched (i.e. multi-armed) or multi-functional, preferably branched polymers, more preferably an 8 arm PAG, most preferably an 8 arm PEG. In polymer chemistry, multiple methods for synthesis can be used to yield a branched polymer. Briefly, there are several general strategies for synthesis, that is, descriptions of how the substituent molecules may be assembled to create the final product without specifying the reactants, reaction conditions, or desired product, which may be employed to create the branched synthetic polymer. Branching can occur by the replacement of a substituent, e.g., a hydrogen atom, on a monomer subunit, by another covalently bonded chain of that polymer; or, in the case of a graft copolymer, by a chain of another type. Branching can also occur by sequential covalent addition of monomers starting at a reactive site on a multifunctional initiator (core) and subsequently at the initiating site at the end of each branched polymer arm until the desired molecular weight is attained and the reaction is quenched. Branching may result from the formation of carbon-carbon or various other types of covalent bonds. Branching by ester and amide bonds is typically by a condensation reaction, producing one molecule of water (or HCl) for each bond formed.

The branching index measures the effect of long-chain branches on the size of a macromolecule in solution. It is defined as g=<sb2>/<s12>, where sb is the mean square radius of gyration of the branched macromolecule in a given solvent, and sl is the mean square radius of gyration of an otherwise identical linear macromolecule in the same solvent at the same temperature. A value greater than 1 indicates an increased radius of gyration due to branching.

There are two features of the PEG for this gel formulation, one being the molecular weight, and the other being the presence of more than two multiple functional groups. In some forms, the multiarmed PEG molecule serves to bind to the binders and connect to at least two crosslinking molecules to crosslink the gel. For example, the multiarm PEG molecule serves to bind to the binder peptides and connect to at least two HA molecules to crosslink the gel. In some forms, by virtue of the use of branched polyalkylene glycol bound to the binders, the hydrogel enriches the binders on the branches of the polyalkylene glycol, providing an environment well-suited to support the growth of organoids from human cells. The synthetic polymer may be linear and multifunctional, containing several (more than 2) reactive functional groups. Linear multifunctional synthetic polymers may be synthesized through a plurality of methods. Synthetic polymers can be functionalized via covalent modification of at one reactive functional group to yield the desired reactive functional group. Alternatively, multifunctional polymers can be obtained through synthesis of a co-polymer including monomers containing the desired functional group as a side chain (i.e. not participating in the polymerization reaction).

A preferred synthetic polymer is polyalkylene glycol, such as polyethylene glycol (“PEG:). Preferred PEGs are branched polymers with at least 3, or 4 arms, for example, 4 arms, 6 arms, or 8 arms. Typically, the arm has a length from the backbone of the polymer to the terminus of the arm between 1 and 100 nm, between 10 and 90 nm, or between 10 and 50 nm, for example, 20 nm.

Representative PEG molecular weights include 10 kDa, 15 kDa, 20 kDa, 30 kDa, 50 kDa, and 100 kDa. In preferred embodiments, the PEG has a molecular weight between about 10 kDa and about 100 kDa, for example, 20 kDa or 40 kDa. PEG of any given molecular weight may vary in other characteristics such as length, density, and branching. In preferred embodiments, the PEG is 8-arm with a MW of 20 kDa. In some embodiments, the PEG is 8-arm with a MW of 40 kDa. In some embodiments, PEGs of various molecular weights can be used, for example, a mixture of 20 kDa PEG and 40 kDa PEG.

In some embodiments, copolymers of PEG or other polymers described above may be used to make the hybrid hydrogel. In certain embodiments, the synthetic polymer used to make the hydrogel may be a co-polymer partially composed of PEG, the other polymers described above, or a combination. The co-polymer may be a random co-polymer, with the monomers distributed randomly throughout the molecule, a block co-polymer, with the monomers distributed in sections of several like monomers clustered together in the chain, or a graft co-polymer, synthesized from a homogenous polymer molecule containing one monomer covalently grafted to a homogenous polymer molecule containing a different monomer.

Assembly of the hydrogel requires mutually reactive functional moieties present on the synthetic polymer, the binders, and the crosslinker (when present) such as peptide or ECM polymer, which participate in covalent bonds that assemble the polymer network of the gel and attach binders within the gel structure. A plurality of reaction chemistries exist that could be used to assemble the hydrogel in the presence of cells, most of which are commonly classified in the literature as ‘click’ chemistry reactions and are classified by Sharpless et al. Agnew. Chemie. 2001 (https://doi.org/10.1002/1521-3773(20010601)40:11%3C2004::AID-ANIE2004%3E3.0.CO;2-5), who list several such reactions. Reactions classified as ‘click’ reactions which may be applicable for this material are those which 1) proceed under mild conditions, i.e. room or physiologic temperatures (~22-37° C.), in aqueous solvent, and at physiologic pH, 2) produce inoffensive or no by-products, and 3) are stereospecific and 4) high-yielding. Some such reactions include thiol-ene reactions, inverse electron-demand Diels Alder reactions, and strain-promoted azide-alkyne click reactions. The choice of chemistry used to assemble the hydrogel precursors will motivate the selection of reactive functional moieties (“linking moieties”) included on the synthetic polymer, ECM polymer, and binders. The choice of chemistry used to assemble the hydrogel precursors will also motivate the selection of particular reaction conditions, such as temperature, pH, and inclusion or exclusion of a catalyst such as a photo initiator (i.e., a molecule which serves as a source of reaction-initiating free radicals when exposed to specific wavelengths of light).

In the preferred embodiments, Michael addition thiol-ene chemistry is used. The participating reactive functional moieties used in the preferred embodiment are thiol (sulfhydryl) groups and an alkene that includes an adjacent electron-withdrawing group, in the preferred embodiment specifically vinyl sulfone. In these forms, the synthetic polymer is bound to binders and optionally crosslinkers via a thioether sulfone bond.

The reactive functional groups can be identified as an electrophilic group or a nucleophilic group, depending on whether within the reaction they serve as an electron acceptor or electron donor, respectively. Exemplary electrophilic groups include, but are not limited to, azide, cyano, trifluoromethyl, vinyl groups or vinyl-containing groups (e.g. vinyl sulfone), maleimide. In some embodiments, the electrophilic group is vinyl sulfone or maleimide. Exemplary nucleophilic groups include, but are not limited to, thiol, hydroxyl, alkoxy or aroxy (e.g., methoxy, benzyloxy), or primary, secondary, or tertiary amine. In some embodiments, the nucleophilic group is a thiol group.

In some embodiments, the synthetic polymers are functionalized with a first reactive functional moiety. The first reactive functional moiety may be present at a side chain functional group of the polymer or at a terminal functional group of the polymer. The molar degree of substitution of the first reactive functional moiety on a synthetic polymer can be varied. For example, at least 1, 2, 3, 4, 5, 6, 7, 8, 10, 12, 16, 20, 24, and 32 first linking moieties may be included per molecule in the polymer, irrespective of the molecular weight of the polymer. The first reactive functional moiety can be a reactive group that reacts with binders functionalized with a second reactive functional moiety to form polymer-binder adducts. Additionally, and alternatively, the first reactive functional moiety can be a reactive group that is involved in crosslinking either between different synthetic polymers or between the synthetic polymers/polymer-binder adducts and the ECM polymer. The first reactive functional moiety can be the same or different in the plurality of synthetic polymers.

In some embodiments, the reactive group can be an electrophilic group or a nucleophilic group. Exemplary electrophilic groups include, but are not limited to, azide, cyano, trifluoromethyl, vinyl groups or vinyl-containing groups (e.g. vinyl sulfone), maleimide. In some embodiments, the electrophilic group is vinyl sulfone or maleimide. Exemplary nucleophilic groups include, but are not limited to, thiol, hydroxyl, alkoxy or aroxy (e.g., methoxy, benzyloxy), or primary, secondary, or tertiary amine. In some embodiments, the nucleophilic group is a thiol group.

In some embodiments, the synthetic polymer has a weight/volume percentage in the hydrogel of between 1% and 50%, between 2% and 25%, or between 2% and 10%, for example, about 2%, about 3%, about 4%, or about 5%.

In some forms, the synthetic polymer, such as branched polyalkylene glycol (e.g., branched PEG), is at a nominal concentration of ≤3 wt % at the time of crosslinking.

ii. Adhesion Ligand Binders

The hydrogel matrix further includes adhesion ligand binding peptides (“adhesion peptides”, “adhesion binders”, “adhesion ligands”, “adhesion ligand binders”, “peptide binders”, or “binders”) specifically covalently attached to the multifunctional linear or branched synthetic polymer. These binders typically contain a second reactive functional moiety. The second reactive functional moiety can be a reactive group that reacts with the first reactive functional moiety to form a polymer-binder adduct. An exemplary second reactive functional moiety is a thiol group. For example, the binders are integrin binding peptides and extracellular protein binding peptides, where each peptide was synthesized to have a free thiol group on a cysteine residue near the N-terminus. Each polymer may have at least one binder incorporated, for example, 1 to 3 binders incorporated, so long as two or more first reactive functional moieties on the multifunctional synthetic polymer remain available to react after binder incorporation.

In some embodiments, adhesion ligand binders are incorporated at the terminus of the arm of a multi-armed PEG such as an 8-arm PEG. The polymer-binder adduct can have an arm length from the polymer branch point to the terminus of the adhesion ligand between 10 and 100 nm. For example, an 8-arm 20 kDa PEG-GFOGER adduct has an arm length about 26 nm (~16 nm PEG and ~10 nm GFOGER). Each arm of the 8-arm 20 kDa PEG may have three GFOGER peptides incorporated. This is typically through formation of triple-helix of the three GFOGER peptides, where one of the peptides binds to one arm. An 8-arm 20 kDa PEG-PHKRGD adduct has an arm length about 21 nm (~16 nm PEG and ~5 nm PHKRGD). For example, each arm of the 8-arm 20 kDa PEG can have one PHKRGD incorporated. The number of binders incorporated on each polymer and the arm length depend on the type of polymer, the molecular weight of polymer, and the type of binder. Such parameters affect the cell's sense of local nano-scale tensile forces and clustering. Typically, only a portion of the arms in the 8-arm polymer are linked to a binder.

The term GFOGER when used herein, bolded and italicized terms e.g., GFOGER as in “GFOGER peptides” or “PEG-GFOGER”, and PHKRGD as in “PHKRGD peptides” refers to a name given to an amino acid sequence or construct. See e.g., Table 1.

In some embodiments, one or more adhesion types of adhesion ligand binders are included in the hybrid hydrogel, for example, one or more integrin binding peptide(s) such as a collagen-I derived peptide, a fibronectin derived peptide, and/or an laminin derived peptide. Typically, each integrin binding peptide independently has a concentration between about 0.1 mM and about 5 mM, between about 0.25 mM and about 3 mM, or between about 0.5 mM and about 3 mM, for example, about 1.5 mM or about 0.6 mM. For example, when the hydrogel precursor composition contains YIGSR (SEQ ID NO:45), it is at a nominal concentration ranging from about 0.1 mM to about 1 mM, such as about 0.6 mM.

In some embodiments, in addition to the integrin peptide(s), two or more extracellular protein-binding peptides (“ECM binders”) are included in the hydrogel, for example, a peptide having affinity to fibronectin (or “FN binder”) and a peptide having affinity to collagen and laminin (or “BM binder”). Preferred ECM binders typically have affinity to fibronectin, collagen type IV, and/or laminin. The ECM binders are typically functional at one end of the peptide to form a covalent or non-covalent bond with the polymer. Preferably, the ECM binders form a covalent bond with the polymer at one of the peptides.

Typically, each extracellular protein-binding peptide has a concentration between about 0.1 mM and about 1 mM, between about 0.1 mM and about 0.5 mM, or between about 0.25 mM and about 0.5 mM, for example, about 0.25 mM or about 0.5 mM.

The molar ratio of the integrin binder(s) (collective molar concentration if more than one type of integrin binder is present) to the ECM binder(s) (collective molar concentration if more than one type of ECM binder is present), is typically greater than 1:1, such as between about 1:1 and about 5:1, between about 1.2:1 and about 5:1, between about 1.5:1 and about 5:1, between about 2:1 and about 5:1, between about 2:1 and about 4:1, or between about 2:1 and about 3:1, such as about 1.5:1, about 2:1, about 2.5:1, about 3:1, about 3.5:1, about 4:1, about 4.5:1, or about 5:1.

Adhesion ligand binders are typically synthetic peptides with affinity to cellular proteins, typically to cell adhesion molecules (CAMs). Cell adhesion molecules include integrins, immunoglobulin superfamily (IgSF) CAMS, cadherins, and selectins. The adhesion ligand binders may derive from proteins of the ECM. The adhesion ligand binders may be linear or branched. In some forms, the adhesion ligand binders have affinity to one or more integrins, and/or to one or more integrin subunits. Adhesion ligand binders typically have two or more functional moieties allowing crosslinking to the PAG and/or crosslinker (e.g., peptides and/or ECM polymers).

The adhesion ligand binders may have a secondary structure. The adhesion ligand binders may be peptides having a single chain amino acid sequence. The adhesion ligand binders may be branched peptides. The adhesion ligand binders may be peptides having a single chain amino acid sequence that self assembles into dimers, trimers, etc. The secondary structure of the dimers and trimers may be any secondary structure for proteins and peptides, including alpha helices, beta sheets, beta turns, or omega loops.

The adhesion ligand binders typically contain a reactive functional moiety at one end of the peptide to form a covalent or non-covalent bond with the polymer. Preferably, the adhesion ligand binders form a covalent bond with the synthetic polymer at one of the peptide side chains.

The adhesion ligand binders may include a motif from any one of fibrillar or non-fibrillar collage. The fibrillar collagen include collagen types I, II, III, V, XI. The non-fibrillar collagen includes FACIT (Fibril Associated Collagens with Interrupted Triple Helices); collagen types IX, XII, XIV, XIX, XXI; short chain type VIII, X; basement membrane (Type IV); multiplexin (Multiple Triple Helix domains with Interruptions) (Type XV, XVIII); MACIT (Membrane Associated Collagens with Interrupted Triple Helices) (Type XIII, XVII); and others (Type VI, VII) (Ricard-Blum, Cold Spring Harb Perspect Biol, 2011; 3:a004978).

Different tissues contain collagen of different type. For example, Type I is common in skin, tendon, vasculature, organs, bone (main component of the organic part of bone); Type II is most common in cartilage (main collagenous component of cartilage); Type III is most common in reticulate (main component of reticular fibers), commonly found alongside type I; Type IV is most common in basal lamina, the epithelium-secreted layer of the basement membrane; and Type V is most common on cell surfaces, hair, and placenta.

The collagen can be of any animal origin or human.

Fibronectin typically exists as a protein dimer, containing two nearly identical polypeptide chains linked by a pair of C-terminal disulfide bonds. Each fibronectin subunit typically has a molecular weight of 230-250 kDa and contains three types of modules: type I, 15 II, and III. All three modules are composed of two anti-parallel β-sheets resulting in a Beta-sandwich; however, type I and type II are stabilized by intra-chain disulfide bonds, while type III modules do not contain any disulfide bonds. The absence of disulfide bonds in type III modules allows them to partially unfold under applied force.

Three regions of variable splicing occur along the length of the fibronectin protomer. One or both of the “extra” type III modules (EIIIA and EIIIB) may be present in cellular fibronectin, but they are never present in plasma fibronectin. A “variable” V-region exists between III14-15 (the 14th and 15th type III module). The V-region structure is different from the type I, II, and III modules, and its presence and length may vary. The V-region contains the binding site for α4β1 integrins. It is present in most cellular fibronectin, but only one of the two subunits in a plasma fibronectin dimer contains a V-region sequence.

The modules are arranged into several functional and protein-binding domains along the length of a fibronectin monomer. There are four fibronectin-binding domains, allowing fibronectin to associate with other fibronectin molecules. One of these fibronectin-binding domains, I1-5, is referred to as the “assembly domain”, and it is required for the initiation of fibronectin matrix assembly. Modules III9-10 correspond to the “cell-binding domain” of fibronectin. The RGD sequence (Arg-Gly-Asp) is located in III10 and is the site of cell attachment via α5β1 and αVβ3 integrins on the cell surface. The “synergy site” is in III9 and has a role in modulating fibronectin's association with a5B1 integrins. Fibronectin also contains domains for fibrin-binding (I1-5, I10-12), collagen-binding (I6-9), fibulin-1-binding (III13-14), heparin-binding and syndecan-binding (III12-14) (Mao et al., Matrix Biology, 24:389-399).

Laminin polyprotein includes fifteen laminin trimers. The laminins are combinations of different alpha-, beta-, and gamma-chains. The five forms of alpha-chains are: LAMA1, LAMA2, LAMA3 (which has three splice forms), LAMA4, LAMA5. The beta-chains include: LAMB1, LAMB2, LAMB3, LAMB4. The gamma-chains are: LAMC1, LAMC2, LAMC3. Laminins were previously numbered as they were discovered, i.e. laminin-1, laminin-2, laminin-3, etc., but the nomenclature was changed to describe which chains are present in each isoform (laminin-111, laminin-211, etc., Aumailley et al., Matrix Biology, 24:326-332 (2005)). The adhesion peptide can be derived from the alpha-, beta-, or gamma-chain of laminin, such as beta-laminin, e.g., laminin β1.

Integrins are cell adhesion molecules and are obligate heterodimers with two subunits: α (alpha) and β (beta). Integrins in mammals have twenty-four α and nine β subunits (Tables 2 and 3). Beta-1 integrins interact with many alpha integrin chains. Integrins work alongside other receptors such as cadherins, the immunoglobulin superfamily cell adhesion molecules, selectins and syndecans, to mediate cell-cell and cell-matrix interaction. Ligands for integrins include fibronectin, vitronectin, collagen and laminin.

Exemplary adhesion ligands include “RGD,” a fibronectin (FN)-derived adhesion peptide containing the canonical RGD motif from the 10th FN type III domain NH2-GCRE-RGDSP(Am) (SEQ ID NO:3); “PHSRN-K-RGD,” a fibronectin-derived adhesion peptide containing both the RGD motif and the PHSRN (SEQ ID NO:2) synergy site from the 9th FN type III repeat in a branched configuration to mimic features of the biophysical presentation in FN, (Ac)PHSRNGGGK-(Ac)GGGERCG-GGRGDSPY(Am) (SEQ ID NO:8); “PHSRN-K-RDG,” a scrambled control sequence where the RGD motif was scrambled reducing integrin binding; (Ac)PHSRNGGGK-GGGERCG(Ac)-GGRDGSPY(Am) (SEQ ID NO:9); “GFOGER,” a collagen I-derived adhesion peptide, NH2-GGYGGGPG(GPP)₅GFOGER(GPP)₅GPC(Am) (SEQ ID NO: 11), “Lam-5,” a laminin 5-derived adhesion peptide, NH2-GCRG-PPFLMLLKGSTR(Am) (SEQ ID NO:13), and “YIGSR,” a peptide derived from the binding motif of laminin, (Ac)-GCREYIGSR-(Am) (SEQ ID NO:44). Note that “(Ac)” is used as shorthand notation for N-terminal acetylation and “(Am)” is used as shorthand notation for C-terminal amidation of the peptides during synthesis.

In some forms, GFOGER (SEQ ID NO:1) may reach maximum efficacy, especially for the culture of epithelial cells. In some forms, inclusion of YIGSR (SEQ ID NO:45) allows for successful culture of endothelial cells, supporting perfusable vasculature.

Binders having affinity to fibronectin are referred to herein as FN binders, and include peptides (NH2)-GCRE-TLQPVYEYMVGV(COOH) (SEQ ID NO:6), and peptides, polypeptides, oligopeptides, or proteins containing the amino acid sequence GCRE (SEQ ID NO:16), and/or TLQPVYEYMVGV (SEQ ID NO:15).

Other exemplary FN binders include peptides with amino acid sequences

(SEQ ID NO: 17)
VPQIHGQNKGNQSFEEDTE,
(SEQ ID NO: 18)
VPQIQGQNKGNQSFEEDTE,
(SEQ ID NO: 19)
VPQIHGQNNGNQSFEEDTE,
(SEQ ID NO: 20)
VPQIQGQNNGNQSFEEDTE,
(SEQ ID NO: 21)
VPQIHGQNIGNQSFEEDTE,
(SEQ ID NO: 22)
VPQIQGQNIGNQSFEEDTE,
(SEQ ID NO: 23)
VPQIAGQNKGNQSFEEDTE,
and
(SEQ ID NO: 24)
VPQIAGQNAGNQSFEEDTE.

Binders having affinity to basement membrane proteins are referred to herein as BM binders. The BM binders typically include synthetic peptides with affinity to one or more of the components of the basement membrane. Exemplary components of the basement membrane include laminin; integrins; nidogens; dystroglycans; collagen types III, IV, and VII; perlacan; heparan sulfate; and fibrillin. The BM binders may have affinity to collagen type IV and laminin.

Exemplary BM binders include the peptide NH2-GCRE-ISAFLGIPFAEPPMGPRRFLPPEPKKP(Am) (SEQ ID NO:5), and peptides, polypeptides, oligopeptides, or proteins containing the amino acid sequence GCRE (SEQ ID NO:16), and/or ISAFLGIPFAEPPMGPRRFLPPEPKKP(Am) (SEQ ID NO:25).

TABLE 1
Exemplary Adhesion Ligand Peptides

| Peptide | Peptide Amino | Linear or |
|---|---|---|
| Name | Acid Sequence | Branched |
| RGD | NH2-GCRE-RGDSP(Am) | linear |
| | (SEQ ID NO: 3) | |
| PHSRN-K- | (Ac)PHSRNGGGK-(Ac)GGGERCG- | branched |
| RGD | GGRGDSPY(Am) (SEQ ID NO: 8) | |
| RDG | NH2-GCRE-RDGSP(Am) | linear |
| | (SEQ ID NO: 26) | |
| PHSRN-K- | (Ac)PHSRNGGGK-GGGERCG(Ac)- | branched |
| RDG | GGRDGSPY(Am) (SEQ ID NO: 9) | |
| GFOGER | NH₂-GGYGGGPG(GPP)₅GFOGER | linear |
| | (GPP)₅GPC(Am) | |
| | (SEQ ID NO: 11) | |
| Lam-5 | NH2-GCRG-PPFLMLLKGSTR(Am) | linear |
| | (SEQ ID NO: 13) | |
| FN-binder | (NH₂)-GCRE-TLQPVYEYMVGV- | linear |
| | (COOH) (SEQ ID NO: 6) | |
| BM-binder | NH₂-GCRE-ISAFLGIPFAEPPMGP | linear |
| | RRFLPPEPKKP(Am) | |
| | (SEQ ID NO: 5) | |

iii. Crosslinkers

Optionally, one or more crosslinkers are included in the synthetic hydrogel, such as extracellular matrix polymers, peptides, and/or small molecules. In some forms, the crosslinkers, such as small molecules or peptides, link the functionalized branches of the polymers together. In some forms, the crosslinkers may link the binding peptides attached to the polymer branches to other binding peptides of other polymers' branches, or to other polymers' functionalized branches. In some forms, the crosslinkers link the functionalized branches of the polymers together.

The crosslinker typically includes two functional ends (bifunctional) for crosslinking. The crosslinkers may be bifunctional small molecules, bifunctional polymers (synthetic or natural, such as ECM polymers), bifunctional oligopeptides (e.g., 2-20 amino acids), bifunctional polypeptides (e.g., >20 amino acids), or any other bifunctional crosslinker.

The crosslinker typically contains a cross linking moiety that reacts with the first linking moiety on the synthetic polymer to initiate cross linking. In some forms, the ratio of cross linking moiety to first linking moiety is between 0.1 and 1, between 0.35 and 1, or between 0.5 and 1, for example, about 0.5. The synthetic hydrogel typically has a cross linking ratio between 10% and 90%, such as between about 20% and 80%, such as between about 20% and 70%. Exemplary crosslinking ratios include about 10%, about 15%, about 20%, about 25%, about 30%, about 35%, about 40%, about 45%, about 50%, about 55%, about 60%, about 65%, about 70%, and about 75%. For example, the synthetic hydrogel has a crosslinking ratio of ≥52%.

The cross-linking density can be adjusted to alter the rate of degradation, the strength, or both, of the synthetic hydrogel. The cross-linking ratio can be adjusted by adjusting the proportion of the synthetic polymers containing reactive groups in the polymers, adjusting the concentration of the cross linkers in the crosslinking reaction, changing the number of reactive arms in the biodegradable polymer, or combinations thereof. The cross-linking ratio can be determined for a crosslinked polymer using any means known in the art, such as the approach described in Wang, et al., Nat. Biotechnol., 20(6):602-606 (2002).

In some forms, the crosslinkers can be a natural polymer or natural peptide, such as a ECM polymer, e.g., hyaluronic acid. In some forms, the crosslinkers can be synthetic peptides. The synthetic peptide can contain a protease-, proteinase-, or transpeptidase-cleavable motif, for example, a motif cleavable by a matrix-metalloproteinase (MMP) and/or a Sortase A transpeptidase. The incorporation of cleavable motifs leads to dissolvable synthetic hydrogels. This allows for recovery of intact cells and cell-secreted metabolites from the hydrogels, which is an important limitation in MATRIGEL® supported organoid growth.

a. Crosslinkable ECM Polymers

In some forms, crosslinkable chemically functionalized ECM polymers such as thiolated HA are used to crosslink the branched PAG such as the 8 arm PEG to form hybrid hydrogels. The multifunctional chemical structure of linear and branched natural polymers found in the ECM enable robust crosslinking with a multifunctional synthetic polymer. According to Flory-Stockmayer gelation theory in polymer chemistry, the greater the number of reactive functional groups per polymer molecule of the reactants included in the gelation reaction, the lower the reaction efficiency required to obtain gelation (“critical conversion”) (Flory, P. J. (1941). “Molecular Size Distribution in Three Dimensional Polymers I. Gelation”. J. Am. Chem. Soc. 63, 3083) (Stockmayer, Walter H. (1944). “Theory of Molecular Size Distribution and Gel Formation in Branched Polymers II. General Cross Linking”. Journal of Chemical Physics. 12, 4, 125).

### ECM Polymers

In the preferred embodiment, the ECM polymer is hyaluronic acid (HA). HA is one of the major constituents of the skin and in extracellular tissues of various parts of the human body and is also found in a variety of other organisms. HA plays a role in several biological processes, including cell growth, migration and differentiation.

HA is a natural linear polysaccharide that has been used extensively in the biomedical field as it is a biocompatible, synthetic, nontoxic and non-immunogenic polymer with high water affinity. HA is formed by repeating units of D-glucuronic acid and N-acetyl-D-glucosamine disaccharide. The presence of multiple carboxylic acid and hydroxyl groups in the HA molecule makes it an ideal candidate for chemical modification. When modified with reactive functional groups such as thiols, HA can be used to crosslink a three-dimensional network of synthetic polymer molecules to form a hydrogel. Chemical crosslinking provides enhanced stability to HA-based materials. The HA is cleaved by naturally occurring hyaluronidases and by oxidation (such as in the presence of reactive oxygen species in injured or inflamed tissue). Hyaluronic acid is commonly used clinically as an excipient for pharmaceutical products and in tissue fillers. HA is readily available commercially and is industrially produced via batch fermentation.

Other ECM polymers include naturally occurring polysaccharide glycosaminoglycans (GAG) such as heparin/heparin sulfate, chondroitin sulfate/dermatan sulfate, and keratan sulfate.

Other ECM include collagen, fibronectin, (tropo)elastin and laminin.

Type I collagen is the most abundant structural protein in soft tissue and one of the earliest ECM components to be identified and isolated. It provides cells with a three-dimensional environment that supports cell growth and influences morphology and function. During tissue repair, collagen promotes cell adhesion and migration by engaging cell surface receptors such as integrin β1 subunit, glycoprotein VI, non-specific receptors, and integrin-like receptors. The triple helix structure of collagen offered several favorable characteristics for engineering cellular functions, including thermal stability and mechanical strength.

Based on the properties mentioned above, type I collagen is the most popular native material for delivering cells or inducing specific cellular behavior for soft tissue regeneration. Collagen can self-assemble under physiological conditions, absorb onto surfaces through nonspecific interactions, and be electro spun into biomaterials. Type I collagen has been used in cell culture as a substrate to study and promote angiogenesis, as a wound dressing to treat burn wounds, as a vehicle to encapsulate stem cells to induce dermal regeneration, and as a scaffold to generate a skin substitute for grafting. Through integrin signaling and growth factor sensing at its interface with cells, a collagen matrix has been shown to maintain mesenchymal stem cells (MSCs) stemness or direct stem cells towards specific lineages for tissue regeneration. The compatibility between collagen matrices and MSCs enabled a collagen scaffold to serve as a cell delivery vehicle for diabetic wounds to promote repair via improved angiogenesis.

Fibronectin promotes cell adhesion and controls cellular functions via peptide domains, including various binding sites for cell surface molecules and other biological molecules such as heparin, fibrin, collagen, and growth factors. The soluble form of fibronectin mainly circulates in plasma. The application of plasma fibronectin is limited to direct application of an aqueous solution, passive absorption, and covalent linkage to the surface of a biomaterial to promote bio-recognition and biocompatibility. These methods lack proper spatial control over deposition. Electro spraying was developed to deposit soluble fibronectin onto the surface of a material with controlled density, thickness, and pore size for optimal cell migration and adhesion.

Other ECM polymers such as (tropo)elastin and laminin present in basement membranes are useful. The different peptide sequences in elastin are employed to generate scaffolding for cell regeneration therapy. These elastin-like polypeptides can self-assemble into a gelatinous material above their transition temperature due to the unique thermo-responsive properties. Alternatively, they can be crosslinked by enzymatic reactions. Previously, such aggregates were applied in cartilage regeneration and were also used to encapsulate adipose-derived stem cells (ASCs) for full thickness dermal wound healing. The effect of an elastin construct on maintaining stemness was also interrogated; the author found that the elasticity and tensegrity of the construct were necessary to provide key mechanical cues for maintaining and expanding hematopoietic stem cells.

Laminin 111, a major epithelial ECM component, has been used as a component of MATRIGEL® to provide a basement membrane-like scaffold. In addition, it has been used in electrospinning to construct fibrous meshes on a charged surface with tunable pore size and fiber diameter. The engineered meshes could maintain ASCs attachment and viability in vitro, and could induce them to develop neurite-like structure. Laminin 111 has also been shown to participate in other biological processes, including angiogenesis and neural differentiation. Laminin 511 was found to maintain the pluripotency of mouse embryonic stem cells through β1 integrins, while laminin 411, 332, and 111 caused either rapid differentiation or cell death.

ECM polymers like collagen, fibronectin, elastin, and laminin are readily accessible commercially, through isolation or transgenic expression. Importantly, the presence of chemical functional groups such as amines, thiols, hydroxyls, and carboxylic acids on these polymers allows for chemical modification with a reactive functional group that can be used to assemble the hydrogel. In some cases, the reactive functional group may be endogenous to the molecule and not require further modification.

b. Peptide Crosslinkers

The crosslinkers may be bifunctional peptide crosslinkers. The peptide crosslinkers may include a cleavable motif containing an amino acid sequence recognized by one or more proteolytic enzymes, such as proteases, proteinases, peptidases, and transpeptidases, such as protein sorting transpeptidases. The peptide crosslinkers with cleavable motifs may be useful for hydrogel dissolution. The dissolution may occur in the presence or absence of a competitive substrate or an acceptor substrate for the enzymes. The competitive substrate may be used to tune the dissolution reaction to a specific region of the hydrogel or slow the reaction time. The acceptor substrate may be used with transpeptidases to complete the cleaving of the crosslinker. Typically, the competitive substrate includes a peptide with an amino acid sequence substantially similar to the cleavable motif. The acceptor substrate may be any sequence of amino acids specific for the transpeptidase reaction, but typically includes at least one, at least two, at least three, at least four, or at least five glycine (G) residues at the N-terminus of the substrate.

The crosslinking peptides may include cleavable motifs specific to a variety of proteolytic enzymes. Optionally, the cleavable motifs are specific to proteinases and/or transpeptidases, such as matrix metalloproteinases and bacterial sortase transpeptidases.

In some forms, the one or more peptide crosslinkers include a sequence cleavable by a protease, including, for example, endoproteases (e.g., serine proteases, cysteine proteases, aspartic acid proteases), and/or metalloproteases (e.g., matrix metalloproteases and A Disintegrin And Metalloproteinase (ADAM)). In some forms, the peptide crosslinkers include sequences cleavable by both proteases and transpeptidases. Typically, the sequences cleavable by both proteases and transpeptidases are different sequences. In some forms, the crosslinkers are peptide crosslinkers having cleavable motifs cleavable by a sortase, a metalloproteinase, or both by a sortase and a metalloproteinase.

Incorporation of the MMP substrate sequences encourages proper cell growth in a 3D hydrogel space by mimicking the ECM. Over time, cells produce proteases (e.g., matrix-metalloproteinase, “MMPs”) that locally degrade the MMP substrate crosslink, which allow the cells to spread or even migrate through the gels (Fonseca et al., Prog. in Poly. Sci. 39(12):2010-29, 2014).

In some embodiments, the crosslinking peptide is (Ac)-GCRDLPRTGGPQGIWGQDRCG(Am) (SEQ ID NO:24).

iv. Additional Factors

The hydrogels may include additional factors, such as growth factors, their fragments, their variants and analogs. Typically, the additional factors are included to further support the growth and/or differentiation of the cells in the enteroids. The additional factors natural or synthetic peptides and small molecules. The additional factors may be co-factors, signaling molecules, and growth factors, including, but not limited to basic fibroblast growth factor (bFGF), FGF-1, FGF-2, FGF-4, FGF-7, FGF-10, transforming growth factor β1 (TGF-β1), activin-A, activin-B, bone morphogenic protein 4 (BMP-4), hepatocyte growth factor (HGF), epidermal growth factor (EGF), R nerve growth factor (PNGF), retinoic acid, interleukin (IL)-3, IL-6, IL-11, Noggin, platelet derived growth factor (PDGF), stem cell factor (SCF), vascular endothelial growth factor (VEGF), Wnt-1, Wnt-2, Wnt-1a, Wnt-5a, Wnt-7a, their variants and analogs. Variants and analogs may include the factor(s) with amino acid modifications, such as EGF with Y13G modification (Reddy et al., Nature Biotechnology, 14:1696-1699 (1996)). See also Cambria, et al., Biomacromolecules 2015; 16(8):2316-26.

### 2. Hydrogel Properties

As shown in Examples 2-5, the hybrid hydrogel supports human endometrial epithelial cell growth in the form of organoids, spherical layers of single cells that expand in a 3D culture matrix. The organoids formed in the synthetic hydrogel show correct polarity, exhibit appropriate architecture, and proliferate. Additionally, other cell types, such as primary human adipocytes and induced pluripotent stem cell-derived neurons, have been cultured in the hybrid hydrogel.

The biomechanical and biological properties of the synthetic hydrogels can be tuned by varying polymer structures, polymer molecular weight, and type of binder, to support growth of a particular type of cells. For example, when ECM polymer is used, the biomechanical and biological properties of the synthetic hydrogels can be tuned by varying the mass ratio of synthetic polymer to ECM polymer, concentration of total polymer, synthetic or ECM polymer molecular weight, and type and concentration of binder, to support growth, proliferation, and/or differentiation of multiple cell types, normal or tumor-derived. For example, hybrid hydrogels formed from 2.5% w/v 8-arm polyethylene glycol (PEG) with 20 kDa molecular weight, integrin binding peptides, FN binders, BM binders, and 0.25% w/v~300 kDa hyaluronic acid (HA) functionalized with thiol groups support human endometrial epithelial cell growth. For example, synthetic hydrogels formed from 8-arm polyethylene glycol (PEG) with 20 kDa or 40 kDa, integrin binding peptides (e.g., collagen derived peptides, fibronectin derived peptides, laminin derived peptides), ECM binders (e.g., FN binders, BM binders), and peptides cleavable by MMP and/or Sortase support endothelial vascular morphogenesis.

The biomechanical properties of the synthetic hydrogels can be measured using relevant features, such as storage modulus, swelling, and pore size. For example, different biomechanical properties (i.e. storage modulus and volumetric swelling factor) were determined with varying concentrations and molecular weights of hyaluronic acid, as described in the examples.

Storage modulus is an indication of hydrogel's ability to store deformation energy in an elastic manner, and is a measurement of what is colloquially referred to as “stiffness”. Storage modulus is directly related to the extent of cross-linking, i.e. the higher the degree of cross-linking, the greater the storage modulus. In some embodiments, the hydrogel has a storage modulus between about 50 Pa and about 2000 Pa, such as between about 50 Pa and about 1800 Pa, between about 50 Pa and about 1500 Pa, between about 50 Pa and 1000 Pa, between about 500 and about 2000 Pa, between about 600 and about 1200 Pa, between about 700 and about 1000 Pa, or between about 600 and about 1050 Pa. The storage modulus can be tuned by varying the polymer structure, polymer molecular weight, and/or type of binders. In some forms, the synthetic hydrogel has a storage modulus of less than or about 150 Pa, less than or about 135 Pa, less than or about 120 Pa, less than or about 100 Pa, or less than or about 75 Pa. In some forms, storage modulus is directly related to the degree of crosslinking, i.e., the higher the number of crosslinking bonds formed between the ECM polymer and the synthetic polymer-binder adducts, the greater the storage modulus. In some embodiments, the hybrid hydrogel has a storage modulus between about 50 Pa and about 2000 Pa, such as between about 50 Pa and about 1800 Pa, between about 50 Pa and about 1500 Pa, between about 50 Pa and 1000 Pa, between about 500 and about 2000 Pa, between about 600 and about 1200 Pa, between about 700 and about 1000 Pa, or between about 600 and about 1050 Pa. The storage modulus can be tuned by the mass ratio of synthetic polymer to ECM polymer, concentration of total polymer, synthetic or ECM polymer molecular weight, and/or type and concentration of binders.

Swelling is inversely related to the degree of crosslinking, i.e. the more crosslinking bonds present in the hydrogel, the more swelling is restricted and the less the hydrogel swells. Hydrogel swelling is typically measured via changes in hydrogel mass or volume over time in aqueous conditions. Final swollen volume is measured at equilibrium, i.e., the point at which the mass and/or volume of the gel is no longer changing. In some embodiments, the synthetic hydrogel exhibits a volumetric swelling ratio (the ratio of gel volume after swelling at equilibrium to the volume of the hydrogel right after crosslinking) of less than or about 3.00, less than or about 2.00, less than or about 1.20, or less than or about 1.00 when exposed to an aqueous fluid. In some embodiments, the synthetic hydrogel exhibits a swelling ratio (the ratio of gel volume after swelling to the volume of the hydrogel right after cross-linking) of less than or about 1.5, less than or about 1.3, less than or about 1.1, less than or about 0.9, or less than or about 0.75 when exposed to a fluid. In some embodiments, the hybrid hydrogel exhibits a swelling ratio (the ratio of gel volume after swelling to the volume of the hydrogel right after cross-linking) of less than or about 500%, less than or about 400%, less than or about 300%, less than or about 250%, less than or about 200%, less than or about 190%, or less than or about 180% when exposed to a fluid.

In some embodiments, the mean pore size of the hybrid hydrogels can be between about 10 nm and about 1 μm, between about 15 nm and about 500 nm, between about 15 nm and about 100 nm, between about 15 nm and about 50 nm, or between about 15 nm and about 35 nm.

Advantages of the hybrid hydrogels include high cell viability comparable to other gel types; usefulness with human and mouse stem cells; usefulness with intestinal stem cells from various regions of the same organ; allows incorporating other cell types (intestinal myofibroblast, immune cells, etc.), non-proteolytic synthesis and breakdown; mechanically robust and easy to tailor to a desired tissue organogenesis; highly reproducible composition and inexpensive and broadly accessible.

In some forms, the synthetic hydrogel has a swelling ratio of less than or about 1.5 and a stiffness (storage modulus) of less than or about 150 Pa. For examples, the synthetic hydrogel has a storage modulus of ≤150 Pa, ≤120 Pa, ≤100 Pa, ≤80 Pa, from 10 Pa to 150 Pa, from 20 Pa to 150 P, or from 50 Pa to 150 Pa; and a swelling ratio of ≤1.5, ≤1.2, from 1 to 1.5 or from 1 to 1.2. Synthetic hydrogels having these swelling ratio and stiffness are particularly suitable for organogenesis in microfluidic devices.

In some forms, as shown in Example 7, the synthetic hydrogel supports perfusable vasculature. Particularly, in some forms, the vascularized synthetic ECM formed using the synthetic hydrogel achieved vessels with open lumens in microfluidic devices. Without being bound to any theories, it is believed that the synthetic hydrogels are fully defined based on the micro-environmental properties and components governing the distinct stages of vessel formation.

### B. Synthetic Hydrogel Precursor Composition

In some forms, the polyalkylene glycol and one or more binders (e.g., one or more integrin binding peptides and one or more ECM binding peptides) in a suitable carrier prior to hydrogel formation is provided as a hydrogel precursor composition. The hydrogel precursor composition can contain any combination of the binders at any of the concentrations described above. In some forms, the hydrogel precursor composition contains one or more integrin binding peptides and one or more ECM binding peptides at a molar ratio greater than 1:1.

In some forms, the integrin peptides included in the hydrogel precursor composition are or contain GFOGER (SEQ ID NO:1), RGD (SEQ ID NO:3), and YIGSR (SEQ ID NO:45); and optionally ECM binding peptides NH₂-GCREISAFLGIPFAEPPMGPRRFLPPEPKKP(Am) (SEQ ID NO:5) and NH₂-GCRE-TLQPVYEYMVGV-COOH (SEQ ID NO:6). In some forms, the hydrogel precursor composition contains YIGSR (SEQ ID NO:45) at a nominal concentration ranging from about 0.1 mM to about 1 mM.

In some forms, the hydrogel precursor composition contains a branched polyalkylene glycol at a nominal concentration of ≤3 wt %. In some forms, the hydrogel precursor composition contains a polymer-binder adduct having one or more integrin peptides and one or more ECM binding peptides bound to the branched pollyalkylene glycol via a suitable linker, such as a thioether sulfone bond.

The carrier of the hydrogel precursor composition can be any suitable buffer, such as acetate buffer, phosphate buffer, citrate buffer, Tris buffer, histidine buffer, glycine buffer, borate buffer, citrated phosphate buffer, ammonium acetate buffer, carbonate buffer, HEPES buffer, MES buffer, TES buffer, BIS buffer, MOPPS buffer, or Goodes buffer, or combinations thereof. In some forms, the buffer is suitable for use with cells, i.e., a cell media. In some forms, the cell media is suitable for survival and proliferation of endothelial cells, such as a mixture of PBS and HEPES at a suitable ratio. More specific exemplary buffers are described in the Examples section below.

### C. Kits

Kits containing the synthetic hydrogel or components for forming the synthetic hydrogel are disclosed. The kits may include every component of the hydrogel in a separate container, or a combination of components in the same container. The kits may also provide the cells to be encapsulated, such as endothelial cells, stromal cells, macrophages, and/or epithelial cells. In some forms, the kit also contains a microfluidic device for forming the hydrogel, performing cell culture for organoid growth. The kits may contain other components, such as buffers. The kits are typically provided with instructions for preparing the hydrogels and encapsulating cells.

The components of the hydrogels provided in the kit may be any one, or a combination of, the polymer(s), the adhesion ligand binders, cross-linker(s), dissolution agent(s), and cells.

An exemplary kit contains the hydrogel precursor composition and a dithiol crosslinking peptide, optionally packaged in separate containers.

### III. Methods of Making the Hydrogels

Methods for forming the synthetic hydrogels are also provided. The formulation methods are highly tailorable and thus can be applied to various biodegradable polymers, binders, and cross linkers. The reactions are performed under cell-compatible conditions. Cell-compatible reactions refer to reactions which are not significantly toxic to living tissue and/or cells. Exemplary cell-compatible reactions include, but are not limited to reactions via thrombin-activated Factor XIIIa under physiological conditions or by another enzymatic addition mechanism known in the art, via Michael addition or click chemistry, or by another mild chemical addition mechanism known in the art.

A preferred method of making the gel is shown in FIG. 1. The multifunctional PAG is reacted with the peptide binders of choice to form a synthetic polymer-binder adduct. The ECM polymer is modified with a reactive functional moiety or most often obtained commercially pre-modified with the reactive functional moiety. The synthetic polymer-binder adduct is then crosslinked with the ECM polymer. Too few reactive functional moieties available on each molecule (either synthetic polymer-binder adduct or ECM polymer) would prevent gelation. In the preferred embodiment, branched PEG is used as the multifunctional PAG and HA is used as the ECM polymer. The space-filling, highly water-absorbing physical properties of HA enables this hybrid hydrogel to use less total mass of polymer compared to hydrogels made with synthetic polymers alone while still maintaining robust mechanical properties.

Although in the preferred embodiment a thiol-vinyl sulfone reaction is used to form the synthetic polymer-binder adducts as well as crosslink via crosslinker, such as peptide or HA, other crosslinking chemistries could be used, such as a stimulus-responsive (e.g., proceeds in the presence of stimulus such as light, pH change, or temperature change) reaction, such as radical-mediated thiol-norbornene click chemistry, or a dynamic covalent chemistry, such as oxime click chemistry. Some examples of dynamic covalent reactions that may be used are reviewed briefly by Uman et al. (Uman et al., J. Applied Polymer Sci., 2019, https://doi.org/10.1002/app.48668).

The use of either a stimulus-responsive or dynamic covalent chemistry could allow the gel to be injectable or extrudable. This is particularly helpful in delivery of cell therapies and in 3D bioprinting. Additionally, the chemical reactions used in the first step, the preparation of the polymer-binder adducts, and in the second step, the crosslinking of the polymer-binder adducts using the crosslinker, such as an ECM polymer, may be the same or different. For example, a thiol-vinyl sulfone Michael addition reaction may be used to react the branched PEG and the peptide binders to form the polymer-binder adducts, and then a different reaction, such as a radical-mediated thiol-norbornene reaction, may be used to crosslink the polymer-binder adducts using the ECM polymer. In that example, the binders may include a vinyl sulfone reactive functional group, the branched PEG may include thiol as a reactive functional group, and the ECM polymer may include norbornene as a reactive functional group. In the preferred embodiment, the two steps are completed using the same reaction, thiol-vinyl sulfone Michael addition chemistry. In the preferred embodiment, the reactive functional group of the branched PEG is vinyl sulfone and the reactive functional groups of both the binders and the crosslinker, such as ECM polymer, are thiols.

Physiological conditions refer to a temperature range of 20-40° C., atmospheric pressure of 1, pH of 6-8. For example, the reactions can be performed at a temperature of 37° C., atmospheric pressure of 1, and pH about 7.4. Typically, the hydrogel can be formed in about 60 min, 40 min, 30 min, 20 min, 10 min, 5 min, 2 min, or 1 min.

### A. Polymer-Binder Adduct Preparation

Typically, the method of forming hybrid hydrogel includes (i) combining one or more synthetic polymers and one or more binders. The synthetic polymer contains a first reactive functional moiety. The first reactive functional moiety may locate in the interior position of the polymer or near or at the terminal positions of the arm of the polymer. The number of first reactive functional moiety on a polymer can be varied but is typically more than 2. For example, multiple first linking moieties may be included in one polymer.

In some embodiments, the first reactive functional moiety of the polymers can be an electrophilic and/or nucleophilic reactive group. The binder contains a second reactive functional moiety. The second reactive functional moiety can be a reactive group that reacts with the first reactive functional moiety to form a polymer-binder adduct. For example, when the synthetic polymers contain an electrophilic group, the second reactive functional moiety can be a nucleophilic group. When the synthetic polymers contain a nucleophilic group, the second reactive functional moiety can be an electrophilic group. Any electrophilic and nucleophilic groups disclosed above can be used. In preferred embodiments, the synthetic polymers can contain vinyl sulfone groups and the binders can contain free thiol groups at the terminus location. In these forms, upon completion of the reaction, the binders are bound to the synthetic polymer via a thioether sulfone bond.

Each polymer may have at least one binder incorporated, for example, 1 or 3 binders incorporated. The incorporated binders may be on the same arm of the synthetic polymer or different arms of the synthetic polymer. Preferably, the binders are incorporated to the terminus of the arm of the synthetic polymer. In some forms, by virtue of the use of branched polyalkylene glycol bound to the binders, the hydrogel enriches the binders on the branches of the polyalkylene glycol, providing an environment well-suited to support the growth of organoids from human cells.

In some embodiments, the hybrid hydrogel contains a weight/volume percentage of polymers between 1% and 10%, between 2% and 25%, or between 2% and 10%, for example, about 2%, about 3%, about 4%, or about 5%. The binders preferably contain integrin binders and extracellular matrix protein binders. The integrin binders may include one or more types of peptides derived from collagen, fibronectin, and/or laminin, such as peptide(s) derived from Collagen Type I, peptide(s) derived from Fibronectin, and peptide(s) derived from laminin. Each type of integrin binding peptides can have a concentration between 0.1 and 5 mM, between 0.25 and 3 mM, or between 0.5 and 3 mM, for example, about 1.5 mM or about 0.6 mM. In some embodiments, two or more extracellular protein-binding peptides are included in the hybrid hydrogel, for example, a peptide having affinity to fibronectin (or “FN binder”) and a peptide having affinity to collagen and laminin (or “BM binder”). Typically, each extracellular protein-binding peptide has a concentration between 0.1 and 1 mM, 0.1 and 0.5 mM, or 0.25 and 0.5 mM, for example, about 0.25 mM or about 0.5 mM.

The molar ratio of the integrin binder(s) (collective molar concentration if more than one type of integrin binder is present) to the ECM binder(s) (collective molar concentration if more than one type of ECM binder is present), is typically greater than 1:1, such as between about 1:1 and about 5:1, between about 1.2:1 and about 5:1, between about 1.5:1 and about 5:1, between about 2:1 and about 5:1, between about 2:1 and about 4:1, between about 2:1 and about 3:1, such as about 1.5:1, about 2:1, about 2.5:1, about 3:1, about 3.5:1, about 4:1, about 4.5:1, or about 5:1.

### B. Crosslinking

The method can additionally include (ii) combining the polymer-binder adduct of step (i) and one or more crosslinkers, wherein the crosslinkers have a crosslinking reactive functional moiety. In some forms, the crosslinker can have two crosslinking moieties, one at each terminus. In some forms, the crosslinker may be chemically-modified ECM polymer, functionalized with multiple (i.e., more than 2) crosslinking reactive functional moieties per molecule. In some forms, the crosslinker may be chemically-modified peptide, functionalized with multiple (i.e., more than 2) crosslinking reactive functional moieties per molecule.

The crosslinking moieties can react with the first linking moieties on the synthetic polymers to perform crosslinking and form the hydrogel. For example, when the synthetic polymers contain an electrophilic group, the crosslinking reactive functional moiety can be a nucleophilic group. When the synthetic polymers contain a nucleophilic group, the crosslinking reactive functional moiety can be an electrophilic group. In preferred embodiments, the synthetic polymers contain vinyl sulfone and the crosslinkers contain dithiols having one thiol group at each terminus, or vice versa. In these forms, upon completion of the reaction, the crosslinkers are bound to the synthetic polymer via a thioether sulfone bond.

In some embodiments, the ratio of crosslinking reactive functional moiety to first reactive functional moiety is between 0.05 and 1, between 0.1 and 1, between 0.35 and 1, or between 0.5 and 1, for example, about 0.5, e.g., 0.52. In preferred embodiments, the ratio of thiol of cross linkers to vinyl sulfone of polymers is between 0.1 and 1, between 0.35 and 1, or between 0.5 and 1, for example, about 0.15 or about 0.5, e.g., 0.52. In some forms, the ratio of crosslinking moiety to first linking moiety is ≥0.52. For example, the ratio of thiol of cross linkers to vinyl sulfone of polymers is ≥0.52.

The concentration of crosslinker, such as ECM polymer, in % weight/volume can vary between 0.10% and 2.0%. In embodiments using hyaluronic acid (HA) as a crosslinking ECM polymer, the average molecular weight of the HA molecule can vary between 10 kDa and 1500 kDa.

Crosslinking can be performed between different synthetic polymers; between different synthetic polymers and crosslinkers; or between the synthetic polymer/polymer-binder adducts and crosslinkers, or a combination thereof. Exemplary crosslinking reactions that are cell-compatible include, but are not limited to, reactions via thrombin-activated Factor XIIIa under physiological conditions or by another enzymatic crosslinking mechanism known in the art, or via Michael addition, click chemistry, or by another mild chemical crosslinking mechanism known in the art.

In preferred embodiments, the crosslinking is performed between the synthetic polymer-binder adducts and crosslinkers. To achieve crosslinking between the synthetic polymers and cross-linkers by the preferred Michael addition, one or more synthetic polymers are functionalized with an electrophilic group and/or a nucleophilic group, whereas the crosslinkers carry a crosslinking reactive functional moiety that reacts with the electrophilic group and/or nucleophilic group on the polymers. For example, the synthetic polymers are modified with an electrophilic group, such as a vinyl sulfone, whereas the crosslinkers carry a nucleophilic group, such as a thiol group. In some embodiments, crosslinking is achieved via FXIIIa-mediated crosslinking between the synthetic polymers and crosslinkers. For example, one or more synthetic polymers are functionalized with a lysine-bearing peptide sequence, whereas the crosslinker ECM polymer is functionalized with or includes a glutamine-bearing peptide sequence. In some embodiments, crosslinking is performed via click chemistry. For example, one or more synthetic polymer in the plurality of synthetic polymers is functionalized with a norbornene or thiol group, whereas cross-linkers contain a thiol or norbornene group.

Alternatively, or additionally, the crosslinking may be performed between polymers. For example, to achieve crosslinking between different polymers by the preferred Michael addition, at least one polymer in a plurality of biodegradable polymers is functionalized with an electrophilic group, such as a vinyl sulfone or maleimide group, whereas at least another biodegradable polymer in the plurality of biodegradable polymers is functionalized with a nucleophilic group, such as a thiol group. In some forms, crosslinking is achieved via FXIIIa-mediated crosslinking between different polymers, at least one polymer in the plurality of biodegradable polymers is functionalized with a lysine-bearing peptide sequence, whereas at least another polymer in the plurality of polymers is functionalized with a glutamine-bearing peptide sequence. In some forms, crosslinking is performed via click chemistry, e.g. norbornene/thiolene click chemistry. At least one polymer in the plurality of polymers is functionalized with a norbornene group, whereas at least another polymer in the plurality of polymers is functionalized with a thiolene.

In some forms, a feature of the hybrid formulation is that the peptides are bound to the multifunctional synthetic polymer such as the multiarm PEG, not the ECM polymer such as the HA. The addition of adhesion peptides directly to HA limits the mobility of the peptides due to reduced motility of the HA molecule backbone and increased steric hindrance compared to peptides bound to PEG.

The following are exemplary selection of components, and ratios thereof:

The preferred formulation is 2.5% w/v PEG-vinyl sulfone, (8-arms=8 vinyl sulfone groups per PEG molecule, 20 kDa molecular weight PEG) which is functionalized via reaction with the following adhesion ligand binders and ECM binders in the presence of 200 mM 4-(2-hydroxyethyl)-1-piperazineethanesulfonic acid (HEPES) and 2× concentration Dulbecco's phosphate buffered saline (PBS) to form a PEG-binder adduct: 1.5 mM GFOGER, 1.5 mM PHSRN-K-RGD, 0.5 mM BM-binder, and 0.5 mM FN-binder. The resulting PEG-binder adducts are crosslinked with 0.25% w/v thiolated HA (~300 kDa, 20-30% degree of substitution).

The concentration and choice of binders used in the formulation may be modified. The concentration and choice of binders used in the formulation may be modified. The preferred formulation has also been used with two additional adhesion ligand binders included in the PEG-binder adduct preparation step: 0.5 mM GCRE-GGIKVAV (SEQ ID NO:28), a sequence derived from the integrin-binding domain on laminin alpha 1; and 0.5 mM GCRE-YIGSR (SEQ ID NO:44), a sequence derived from the integrin-binding domain on laminin beta 1.

The preferred maximum amount of adhesion ligand peptides that can be added to the preferred formulation (without increasing the concentration of multiarm PEG beyond 2.5% w/v) is 5 mM total. It has been found that for a gel made with 2.5% w/v PEG-vinyl sulfone and 0.25% w/v thiolated HA, only about 1 mM of vinyl sulfone groups are required to react with the HA in order to form a gel. In the preferred embodiment, the concentration of vinyl sulfone reactive functional groups that remain after PEG-binder adduct preparation is in excess of 1 mM (5-6 mM) in order to ensure gelation is very consistent even with mild variability in the reagents.

The concentration of PEG and HA may be modified from the preferred formulation to control the stiffness and gelation speed of the resulting gel. A softer formulation relative to the preferred formulation has been used for adipocyte culture, which maintains the 10:1 ratio 15 of PEG to HA at a lower concentration of both (1.67% w/v PEG-VS+0.167% thiolated HA), but with the same concentration of all the peptide binders.

A stiffer gel with a 5:1 ratio of PEG to HA (2.5% w/v PEG+0.50% thiolated HA) and the same concentration of peptide binders has also been used. This yields a gel approximately 10-fold stiffer than the ‘standard’ formulation. The ‘stiff’ gel formulation (with a 5:1 PEG to HA) gels about twice as fast as the ‘standard’ formulation. As the PEG concentration increases relative to the HA concentration, the gel becomes less stiff and slower to gel. For comparison, a gel with a 6.6:1 ratio of PEG to HA (2.5% w/v PEG+0.38% w/v thiolated HA) with the same concentration of the peptide binders has also been used. This formulation yields a gel approximately 5-fold stiffer than the ‘standard’ formulation, which is less stiff than the formulation made with a 5:1 ratio of PEG to HA. The 6.6:1 PEG:HA gel formulation was also observed to gel more slowly than the 5:1 PEG:HA gel formulation.

### C. Combining Cells

In some forms, the method further includes (ia) combining cells with the polymer-binder adduct, optionally before addition of the crosslinker(s). For example, the method additionally includes combining cells, one or more inhibitors of dissociation-induced apoptosis, and the polymer-binder adduct of the first step. The incorporation of inhibitors can be important for preventing death of encapsulated cells, especially for human organoid culture. In some embodiments, the inhibitor is at a concentration between 1 nM and 1 mM, between 10 nM and 100 μM, or between 100 nM and 50 μM, for example, about 10 μM.

In these forms, crosslinking of the hydrogel is performed in the presence of cells to be cultured within the hydrogel. This way the cells or cell aggregates are encapsulated by the formed hydrogel matrix, i.e. are residing in a distinct cell culture microenvironment. Cross-linking of the hydrogel is preferably performed in the presence of cells to be cultured within the hydrogel. This way the cells or cell aggregates are encapsulated by the formed hydrogel matrix, i.e. are residing in a distinct cell culture microenvironment. Alternatively, the cells and inhibitors of dissociation-induced apoptosis can be combined with the hydrogel formed after crosslinking.

The cells are typically incorporated at a cell density between about 1×10⁵ cells/ml and about 1×10⁹ cells/ml as the final density in the polymer-binder-crosslinker solution (“pre-gel solution”) at the time of crosslinking. Exemplary suitable ranges for the cell density in the pre-gel solution include between about 1×10⁵ cells/ml and about 1×10⁸ cells/ml, between about 5×10⁵ cells/ml and about 5×10⁸ cells/ml, between about 5×10⁵ cells/ml and about 1×10⁸ cells/ml, preferably between about 1×10⁶ cells/ml and about 1×10⁸ cells/ml, more preferably between about 1×10⁶ cells/ml and about 1×10⁷ cells/ml.

### IV. Methods of Using the Hybrid Hydrogels

Hydrogels for organogenesis from mammalian cells, preferably biopsied human cells, and especially epithelial cells and endothelial cells, have been developed. The hybrid hydrogels are suitable for 3D cell culture, organoid formation, drug discovery, inter-organ interaction characterization, and multi-organ drug responses. The hydrogels are tunable matrices with components selected to support growth of cells of different origins (e.g., cells of mouse and human origin) and from different organs and tissues (e.g., endometrial cells, endothelial cells, or intestinal cells). The hydrogels provide a matrix where the complex cell-matrix interactions can be controlled, which are not possible with complex natural hydrogels.

In some forms, the hybrid hydrogels are particularly suitable for supporting culture of primary human cells and interrogating the biological response of cells in culture to hyaluronic acid. These are especially useful for culturing endometrial epithelial cells and organoids thereof, as well as some other types such as primary human adipocytes and neural cells.

In some forms, the synthetic hydrogels have properties (such as a storage modulus of less than or about 150 Pa and a swelling ratio of less than or about 1.5) that make it particularly suitable for microfluidic organogenesis. In some forms, the synthetic hydrogels is suitable for vessel formation and vascular morphogenesis, drug discovery, disease modeling, and coculturing of vasculature and organoids. In some forms, the synthetic hydrogels are particularly suitable for supporting vascular morphogenesis and disease modeling for human endothelial cells. Through a semi-empirical approach, the biomechanical properties of the hydrogels can be tailored to support vascular morphogenesis to facilitate vascularized tissue models.

In some forms, methods of using the precursor compositions and hydrogels, for organogenesis (such as vascularized organogenesis), involves the following steps: (i) incorporating cells into a hydrogel precursor composition; (ii) mixing the hydrogel precursor composition containing cells with a crosslinker (such as dithiol crosslinking peptide or dithiol ECM polymer) to form a pre-gel solution. The pre-gel solution is then maintained at a suitable temperature for a time period sufficient to form the hydrogel that incorporates the cells therein.

In some forms, vascularized organogenesis is performed in a microfluidic device. In these forms, the method further includes: (iii) loading the pre-gel solution into a microfluidic device. The pre-gel solution loaded microfluidic device is then maintained at a suitable temperature for a time period sufficient to form the hydrogel that incorporates the cells therein, in the microfluidic device.

### A. Incorporation of Cells

The hydrogel precursor composition is as described above, i.e., containing polymer(s) and binders in a carrier. The binders and polymer(s) in the synthetic hydrogel precursor composition are typically allowed to react to form polymer-binder adduct before incorporation of the cells. In some forms, the hydrogel precursor composition is added directly to a cell pellet after centrifugation to remove cell media. When necessary, the cell pellet is resuspended in the synthetic hydrogel precursor composition using non-invasive mixing techniques. In some forms, a pipette is used to resuspend the cell pellet. The cells can be incorporated into the synthetic hydrogel precursor compositions at any temperature safe for cells, for example about 20° C. to about 40° C. or about 20° C. to about 37° C.

Any cell type suitable for organoid formation can be used with the methods, such as endothelial cells, mural cells, stem cells, or epithelial cells. For vascularized organogenesis, the methods use human endothelial cells. In some forms, the methods use human epithelial cells, such as endometrial epithelial cells. Optionally, one or more additional cell types and/or organoids can be used in the method. Examples of additional cell types include, but are not limited to, immune cells, stromal cells, and epithelial cells. In some forms, the method only uses endothelial cells. In some forms, the method uses endothelial cells with stromal cells, macrophages, epithelial cells, or organoids, or a combination thereof.

The cells are typically incorporated at a cell density between about 1×10⁵ cells/ml and about 1×10⁹ cells/ml as the density in the polymer-binder-crosslinker solution (i.e., pre-gel solution). Exemplary ranges of the cell density in the pre-gel solution include between about 1×10⁵ cells/ml and about 1×10⁸ cells/ml, between about 5×10⁵ cells/ml and about 5×10⁸ cells/ml, between about 5×10⁵ cells/ml and about 1×10⁸ cells/ml, between about 1×10⁶ cells/ml and about 1×10⁸ cells/ml, or between about 1×10⁶ cells/ml and about 1×10⁷ cells/ml.

### 1. Exemplary Method for Cell Encapsulation

In some examples, the cells are incorporated with the synthetic polymer-binder adducts at the time of addition of the crosslinker ECM polymer. Certain cell types, such as endometrial epithelial cells, are incorporated in the presence of an apoptosis inhibitor. The cells may be at any suitable density (that is, concentration of cells per unit volume of gel) and are not harmed by the subsequent crosslinking reaction. The crosslinking step occurs at physiological conditions, e.g., at about neutral pH, and 37° C.

An exemplary method includes the following steps. Hydrogels may be fabricated as gel droplets in the base of a non-tissue culture treated polystyrene 96-well plate using Michael-type reaction chemistry. First, the reaction mixture for a concentrated PEG-binder adduct solution was made. 5% w/v 20 kDa, 8-arm poly(ethylene glycol)-vinyl sulfone (PEG-VS), 200 mM HEPES/2×PBS, and the peptide binders 3.0 mM PHSRN-K-RGD, 3.0 mM GFOGER, 1.0 mM BM-binder, and 1.0 mM FN-binder were combined in a microtube and vortexed to mix. The mixture was allowed to react at room temperature for 30 min to form the PEG-binder adduct. After 30 min, the concentrated PEG-binder adduct solution was moved to ice until further use.

The cells were detached from their culture surface, such as a T75 culture flask, using a dissociation reagent such as trypsin/EDTA. The cells were collected and pelleted via centrifugation, then the supernatant containing trypsin/EDTA was removed. The cells were then resuspended in growth media, such as DMEM/F12/FBS, and counted. The cell solution is then split into microtube aliquots, with the number of cells per tube determined by the desired final cell density and batch size (i.e., final volume of gel) for the gel samples. An exemplary cell suspension aliquot would contain 3×10⁵ cells to produce a final volume of 30 μl of gel with a final cell density of 1×10⁷ cells/ml of gel. The aliquots of cells were then centrifuged to obtain cell pellets.

To encapsulate the cells in the gel, the supernatant was carefully and completely removed from one cell pellet. The cells were resuspended in sufficient sterile PBS to achieve the desired final volume of the mixture after adding the PEG-binder adduct solution and the thiolated HA (HA-SH) stock solution. The concentrated PEG-binder adduct solution was then added to the cell suspension so that the PEG-binder adduct solution will be diluted 2-fold in the final pre-gel mixture. The PEG-binder adduct solution is diluted in PBS before the addition of the HA-SH stock solution, because if the two are mixed together without dilution the mixture will gel instantaneously, presumably via physical interactions. Pipette the mixture up and down repeatedly to mix. The HA-SH stock solution was added to the mixture with the cells and pipetted up and down repeatedly to form the pre-gel mixture. The final concentrations of each component in the pre-gel mixture were: 2.5% w/v PEG-VS, 100 mM HEPES/1×PBS, 1.5 mM PHSRN-K-RGD, 1.5 mM GFOGER, 0.5 mM BM-binder, 0.5 mM FN-binder, and 0.25% w/v HA-SH. Immediately after combining the HA-SH, 3 μl droplets of pre-gel mixture were deposited per well in a 96-well, non-tissue culture-treated polystyrene plate. The well plate was moved to a humidified incubator at 37° C. and 5% C02 for 30 min to polymerize.

After gelation is complete, growth media, such as DMEM/F12/FBS, may be added to the apical (top) (100 μL) and basolateral (bottom) (600 μL) sides of the TRANSWELL® to achieve hydrostatic equilibrium. Cultures are typically maintained in a humidified incubator at 37° C., 95% air, 5% CO₂.

B. Mixing with Crosslinkers

The crosslinker for forming the synthetic hydrogel can be any one of those described above, such as a peptide crosslinker or an ECM polymer. In some forms, the peptide crosslinker is a dithiol crosslinking peptide. In some forms, the peptide crosslinker has or contains the sequence (Ac)-GCRDLPRTGGPQGIWGQDRCG(Am) (SEQ ID NO:24). In some forms, the ECM polymer crosslinker is hyaluronic acid.

Crosslinking in the pre-gel solution is preferably performed in the presence of cells to be cultured within the hydrogel. When cells are being used, the crosslinker, such as peptide crosslinker or ECM polymer, is typically added to the synthetic hydrogel precursor composition after incorporation of the cells to form a pre-gel solution. This way the cells or cell aggregates are encapsulated by the formed hydrogel matrix, i.e. are residing in a distinct cell culture microenvironment.

In some forms, the cells are incorporated while mixing the crosslinkers, such as peptide crosslinkers or ECM polymers, with the synthetic hydrogel precursor composition to create the pre-gel solution. In some forms, the pre-gel solution is loaded into a microfluidic device to allow gelation.

### C. Hydrogel Formation

The method includes placing the pre-gel solution or the microfluidic device loaded with the pre-gel solution at a suitable temperature for a time period sufficient to allow gelation to form the hydrogel. Generally, temperature suitable for gelation of the pre-gel solution is between about 30° C. to about 40° C., between about 30° C. to about 37° C., or between about 35° C. to about 40° C. The gelation process is typically fast, e.g., completed within 3 hours, such as within 2 hours, within 1 hour, or within 30 mins. For example, the gelation is performed at about 37° C. for about 30 min.

In some forms, the pre-gel solution or the microfluidic device loaded with the pre-gel solution is placed in an incubator. The incubator is optionally humidified and set at a temperature elevated from room temperature, for example, between about 30° C. to about 40° C., between about 30° C. to about 37° C., or between about 35° C. to about 40° C. The pre-gel solution is incubated for a sufficient amount of time to form the hydrogel, for example, within 5 min, within 15 min, within 30 min, within 45 min, within 1 hour, within 1.5 hours, within 2 hours, or within 3 hours.

In some forms, the synthetic hydrogel formed using the above-described components has a storage modulus and swelling ratio suitable for vasculature in a microfluidic device. For examples, the synthetic hydrogel has a storage modulus of ≤150 Pa, ≤120 Pa, ≤100 Pa, ≤80 Pa, from 10 Pa to 150 Pa, from 20 Pa to 150 P, or from 50 Pa to 150 Pa; and a swelling ratio of ≤1.5, ≤1.2, from 1 to 1.5 or from 1 to 1.2.

In some forms, the synthetic hydrogel exhibits a swelling ratio suitable for use to form vasculature in microfluidic device of less than or about 1.5, less than or about 1.3, less than or about 1.1, less than or about 0.9, or less than or about 0.75.

In preferred forms, the synthetic hydrogel for vasculature formation has a storage modulus of less than or about 150 Pa and a swelling ratio of less than or about 1.5.

### D. Vascularization of Synthetic Hydrogel Compositions

After gelation is complete, the synthetic hydrogel is exposed to a cell culture media for a time period sufficient to form a tissue, such as a vascular tissue. In some forms, the cell culture media is added to the synthetic hydrogel containing desired cells, such as endothelial cells, in a microfluidic device.

In some forms, the cell culture media is added into the microfluidic device at a flow rate and pressure drop that is close to the physiological flow rate and shear stress experienced in a human vessel. For example, the cell culture media is added to a microfluidic channel, optionally pumped into the microfluidic channel, at a flow rate ranging from about 0.1 μL/s to about 10 μL/s, from about 0.5 μL/s to about 5 μL/s, from about 0.5 μL/s to about 2 μL/s, such as about 1 μL/s; and/or with a pressure drop across the channel ranging from about 10 mm H₂O to about 50 mm H₂O, from about 15 mm H₂O to about 35 mm H₂O, or from about 20 mm H₂O to about 30 mm H₂O, such as about 25 mm H₂O.

In some forms, the cell culture media continuously flows through the synthetic hydrogel for a time period sufficient to form a vascular tissue, for example, 1 hour, 3 hours, 6 hours, 12 hours, 1 day, 2 day, 3 days, 4 days, 5 days, 6 days, 7 days, 10 days, 15 days, 20 days, or 30 days. The formation of vascular tissue in the microfluidic device using the synthetic hydrogel can be performed at a suitable temperature for cell culture, such as between about 25° C. to about 37° C.

### E. Optional Steps

Materials commonly used for microfluidic device fabrication, such as PDMS, readily absorb hormones. Therefore, in some forms, a presoaking step is included to saturate the microfluidic device with media containing steroid hormones prior to loading of the pre-gel solution. In some forms, an estrogen hormone is used for presoaking the microfluidic device. In some forms, estradiol is used for presoaking the microfluidic device.

To presoak a microfluidic device, the microfluidic device is fully immersed in a media containing suitable hormones (such as estradiol) at a temperature for a time period sufficient to saturate the microfluidic device with the hormones (such as estradiol) and drying the microfluidic device saturated with the hormones. The media generally contains the hormones at a concentration ranging from about 0.1 nM to about 1000 nM, about 10 nM to about 1000 nM, about 10 nM to about 100 nM, about 1 nM to about 10 nM.

In some forms, the media containing the hormones is maintained at a temperature that does not damage the microfluidic device, such as between about 0° C. to about 60° C., between about 4° C. to about 60° C., between about 20° C. to about 60° C., between about 25° C. to about 60° C., or between about 37° C. to about 60° C. In some forms, the microfluidic device is immersed in the media containing the hormones for a time period ranging from 15 min to 7 days, from 15 min to 24 hours, from 1 hour to 24 hours, such as 15 min, 30 min, 45 min, 1 hour, 3 hours, 6 hours, 12 hours, 1 day, 2 day, 3 days, 4 days, 5 days, 6 days, or 7 days.

The device can be dried at a suitable temperature and for a time duration to remove the remaining media. For example, the media can be dried at a temperature between about 20° C. to about 60° C., between about 25° C. to about 60° C., or between about 37° C. to about 60° C. for a time period range from 15 mins to 24 hours, from 1 hour to 24 hours, from 5 hours to 24 hours, such as about 15 min, 30 min, 45 min, 1 hour, 3 hours, 6 hours, 12 hours, or 1 day. The drying can be performed under vacuum. For example, the drying step is performed in a 37° C. vacuum oven overnight.

### F. Cells, Tissues, and Organs

Typically, the hydrogels support attachment, growth, and differentiation of primary cells, that is, cells isolated directly from living organs and tissues. The hydrogels may also be used to expand organoids, that is, grow from single cells to multicellular structures with a well-defined composition, structure, and morphology, or culture organoids derived from primary tissue, that is, multicellular structures with heterogenous cell composition. In some forms, the hydrogels are suitable for encapsulating cells derived from stem cells, such as differentiated induced pluripotent stem cells, as well as established cell lines. In some forms, the hydrogels are suitable for encapsulating cells that are able to undergo vascular tissue formation, such as endothelial cells.

Generally, the cells in the hydrogels form tissues and organs with differentiation, architecture, cellular composition and cellular organization characteristic of epithelial tissues, gastric tissues, intestinal tissues, endometrial tissues, cardiac tissues, vascular tissues, renal tissues, pulmonary tissues, immune cell interactions, neuronal tissues, osteochondral tissues, and muscle tissues.

The cells or organoids formed in the hydrogels may be suitable for forming or studying the organs, tissues, and their interactions in the reproductive system, cardio-vascular system, renal system, pulmonary system, digestive system, immune system, nervous system, and/or musculoskeletal system.

### 1. Vascular Tissue

For vascularized organogenesis, the synthetic hydrogel contains cells that are able to undergo vascular tissue formation. By incorporating suitable endothelial cells optionally in combination with other types of cells and/or organoids in the hydrogel, the methods can form a variety of different vascular tissues, such as vasculature with open lumen and vascular lesions. Exemplary cells of other types are described above, such as stromal cells, macrophages, endometrial epithelial cells, etc. Exemplary organoids include, but are not limited to, endometrial epithelial organoids.

For example, endothelial cells can be added in combination with stromal cells and endometrial epithelial cells to form vascular lesions. In some forms, the synthetic hydrogel composition forms uterine bed vasculature using uterine endothelial cells and stromal cells. In some forms, the synthetic hydrogel forms endometriosis lesions using endothelial cells, stromal cells, and endometrial epithelial organoids.

In some forms, vascular tissue formation is facilitated by changes in pressure drop and flow rate of cell culture media exposed to the synthetic hydrogel. In some forms, the flow rate and pressure drop of the cell culture media are controlled in a microfluidic device. For example, the synthetic hydrogel is exposed to a cell culture media at a flow rate ranging from about 0.1 μL/s to about 10 μL/s, from about 0.5 μL/s to about 5 μL/s, from about 0.5 μL/s to about 2 μL/s, such as about 1 μL/s; and with a pressure drop ranging from about 10 mm H₂O to about 50 mm H₂O, from about 15 mm H₂O to about 35 mm H₂O, or from about 20 mm H₂O to about 30 mm H₂O, such as about 25 mm H₂O.

### G. Hydrogel Dissolution

The hydrogels with cells or organoids are formed of materials permitting direct assaying of the cells or organoids without the need to remove the hydrogel components. The hydrogels with cells or organoids may be subject to dissolution to remove a portion, or substantially all of the components of the hydrogel.

The removal of hydrogel components may occur through hydrogel dissolution. Hydrogel dissolution includes incubating the hybrid hydrogels with cells or organoids in the presence of dissolution agents such as hyaluronidase. Typically, the incubation is for at least about 5 min, about 10 min, about 15 min, about 20 min, about 25 min, about 30 min, about 35 min, about 40 min, about 45 min, about 50 min, about 55 min, or about 60 min. In preferred embodiments, the dissolution is complete within about 10 min, about 15 min, about 20 min, about 25 min, about 30 min, about 35 min, about 40 min, about 45 min. Typically, the incubation occurs at physiological conditions, such as 37° C., 5% CO₂, humidified atmosphere, and includes growth media used to support survival of the cells or organoids.

Dissolution agents typically include enzymes, such as hydrolases such as hyaluronidase, as well as competitive substrates and acceptor substrates.

The dissolution agents may be in a solution at a concentration between about 0.01 μM and about 100 μM, such as between about 0.1 μM and about 100 μM, between about 1 μM and about 100 μM, between about 1 μM and about 75 μM, preferably between about 1 μM and about 50 μM, most preferably about 10 μM, about 30 μM, about 50 μM. Competitive substrates and/or acceptor substrates may be used at similar concentrations, and may be added together with the enzyme(s), or after the addition of the enzyme(s).

### H. Commercial Applications

The most immediate application of this platform is for use in cell and organoid culture in research. The most commonly used hydrogel for 3D culture on the market today is MATRIGEL® (Corning), which is derived from the basement membrane proteins secreted by Engelbreth-Holm-Swarm murine sarcoma cells. Several companies now offer their own version of basement-membrane based protein hydrogel (such as GELTREX® from Thermo Fisher Scientific and ULTIMATRIX from Bio-Techne). However, these materials obtained from different manufacturers but are not interchangeable, and even users of MATRIGEL® alone report significant lot-to-lot variability. Since the gel is poorly characterized and a natural product, it is difficult to identify the source of variability. The PEG-peptide-HA gel is well characterized and has exhibited highly consistent performance in the culture of endometrial epithelial organoids across lots of PEG, peptides, and HA and with cells sourced from multiple donors.

Natural polymer hydrogels, such as collagen gels or polysaccharide (alginate, cellulose, dextran) based gels have been developed as an alternative, but have been used significantly less commonly compared to MATRIGEL®. These gels have a more controlled composition, but have limited physiological relevance and tend to perform poorly for primary cells in comparison to MATRIGEL®. The hybrid hydrogel described herein is well characterized and has exhibited highly consistent performance in the culture of endometrial epithelial organoids across lots of PEG, peptides, and HA and with cells sourced from multiple donors. Additionally, the hybrid hydrogel uses a combination of synthetic peptides and HA to imitate both protein and glycan molecular features of the extracellular matrix microenvironment.

Synthetic polymer hydrogels have been developed, but there are few commercially available options at present. A synthetic, PEG-based gel platform described in PCT/US2020/044067 “Synthetic Hydrogels for Organogenesis” by Massachusetts Institute of Technology, has demonstrated similar success for endometrial epithelial organoid expansion and culture. However, the hybrid hydrogel has demonstrated advantages over this platform. The first, and most important, is that it can be used to interrogate the biological effects of HA in the tissue microenvironment. Second, by crosslinking the gel with a multifunctional thiolated HA as opposed to a bifunctional peptide crosslinker, the molecular architecture allows for less sensitivity to gel stoichiometry. In terms of performance, this translates to less lot-to-lot and user-to-user variability. Third, preliminary data suggests that certain cell types, such as primary adipocytes, exhibit better viability in an environment containing HA. Fourth, hybrid hydrogels exhibit minimal swelling, which makes them especially well-suited for use in microfluidic platforms.

The crosslinking chemistry of this gel in its preferred embodiment (vinyl sulfone Michael addition) also allows for some degree of control of gelation time through control of the pH of the pre-gel solution. The crosslinking chemistry used to assemble the hydrogel could be adapted for extrusion or injection. Additionally, the protein-free, xeno-free nature of the gel is beneficial for human use. Xenogenic protein products present complications when translating to use in human patients because of potential antigenicity as well as the potential transmission of infectious agents from animals to humans. Hyaluronic acid is a natural product, but is commonly produced via fermentation and is already commonly used as a drug delivery excipient and as tissue fillers for human patients. Furthermore, hyaluronic acid has a simple molecular structure that is conserved throughout the animal kingdom, i.e., pure hyaluronic acid from an animal such as a mouse is chemically indistinguishable from pure hyaluronic acid in a human. For all these reasons, while the primary application is for use in 3D culture systems, this hydrogel has potential applications in drug and cell therapy delivery, tissue engineering, and regenerative medicine.

While the monofunctional bioactive (cell-adhesive and protein-adhesive) peptide binders are the same as the material described in PCT/US2020/044067, the fundamental structure of the gel is quite different and required development from scratch. First, the hybrid hydrogel in its preferred embodiment uses HA, with several thiols per molecule to crosslink the 8-arm PEG molecules together, in place of the enzymatically degradable peptide with one thiol at each end in the material described in PCT/US2020/044067. Using two molecules with several reacting groups on each as reactants when forming the hydrogel makes the hydrogel less sensitive to differences in the ratio between the reacting groups. Also, this strategy essentially the reverse of other published HA-based hydrogels. The standard practice is to use a difunctional PEG to crosslink the HA molecules (which are most of the polymer included in the hydrogel by mass) together. Instead, the hybrid hydrogels use a smaller amount of multi-functional HA to crosslink PEG together. This is because the mass ratio between the PEG and the HA is extremely important and requires thorough optimization as the physical interactions of two different polymers in combination in a hydrogel are difficult to predict.

Through a semi-empirical approach, the biomechanical properties of the hydrogels can be tailored to support the culture of cells and organoids from various organs, or regions of organs. The examples below show the growth of primary human endometrial epithelial cells, primary human adipocytes, and human neurons derived from induced pluripotent stem cells (iPSC) in the hybrid hydrogels. They also demonstrate the tuning of the biomechanical properties of the hydrogels by tuning the mass ratio of synthetic polymer to ECM polymer, concentration of total polymer, and ECM polymer molecular weight.

The hybrid hydrogels are also useful in drug discovery. The examples show that organoids emerging in the hybrid hydrogels respond to drug stimulation to a similar extent as organoids emerging in MATRIGEL®

The disclosed compositions and methods can be further understood through the following numbered paragraphs.

Paragraph 1. A formulation for a matrix for culturing cells comprising
a branched poly(alkyl oxide) polymers such as an 8-arm, 20 kDa PEG macromers (“PEG-VS”) synthesized with a tripentaerythritol core and functionalized with vinyl sulfone terminal groups (molar degree of substitution <90%),
thiol-functionalized extracellular matrix polymer, preferably hyaluronic acid (HA-SH), preferably with a molecular weight of approximately 300 kDa and molar degree of substitution of approximately 20-30, preferably thiolated at the carboxylic acid residues of the D-glucuronic acid portion of the repeating units of the polymer, preferably consisting of one D-glucuronic acid group and one N-acetyl-D-glucosamine group.

Paragraph 2. The formulation of paragraph 1 further comprising an aqueous solution, preferably sterile phosphate buffered saline (PBS), pH 7.4 or Dulbecco's phosphate buffered saline and 1 M 4-(2-hydroxyethyl)-1-piperazineethanesulfonic acid (HEPES) pH 7.8-8.2.

Paragraph 3. The formulation of paragraph 1 or 2 comprising monofunctional or bifunctional peptides containing one or two thiol groups or functional groups reactive with or bound to the reactive functional group present on the branched poly(alkyl oxide) species.

Paragraph 4. The formulation of any of paragraphs 1-3 wherein the peptides are selected from the group consisting of:
“LW,” a peptide with one cysteine residue at each end to facilitate crosslinking of PEG-VS macromers, containing a MMP-labile domain (GPQGIWGQ) (SEQ ID NO:43) and a Sortase-labile domain (LPRTG) (SEQ ID NO:27).
“PHSRN-K-RGD,” a fibronectin-derived peptide containing the RGD motif and the PHSRN (SEQ ID NO:2) synergy site from the 9th fibronectin type III repeat in a branched configuration;
“GFOGER,” a collagen I-derived peptide presented as a triple helix;
“FN-binder,” a peptide with non-covalent binding affinity for fibronectin; and
“BM-binder,” a peptide with non-covalent binding affinity for collagen IV and laminin.

Paragraph 5. The formulation of any of paragraphs 1-4 wherein the polymers are crosslinked to form a matrix.

Paragraph 6. The formulation of paragraph 5 further comprising cells.

Paragraph 7. A method of making the formulation of any of paragraphs 1-6 comprising providing the polyalkylene oxide and extracellular matrix polymers and adhesion ligand peptides and crosslinking.

Paragraph The method of paragraph 7 further comprising providing cells.

Paragraph 9. The method of paragraph 8 wherein the cells are endothelial cells, smooth muscle cells, epithelial cells, and organoid forming cells.

Paragraph 10. The method of paragraph 9 further comprising testing drugs or hormones on the cells in the matrix.

The disclosed compositions and methods can be further understood through the following numbered paragraphs.

Paragraph 1. A synthetic hydrogel cell culture matrix comprising:
a branched polyalkylene glycol;
one or more integrin peptides; and
one or more extracellular matrix (ECM) binding peptides,
wherein the one or more integrin peptides and the one or more ECM binding peptides are bound to the branched polyalkylene glycol,
wherein the one or more integrin peptides comprise GFOGER (SEQ ID NO:1), RGD (SEQ ID NO:3), and YIGSR (SEQ ID NO:45), and
wherein the molar ratio of the one or more integrin peptides to the one or more ECM binding peptides is greater than 1:1.

Paragraph 2. The synthetic hydrogel cell culture matrix of paragraph 1, having a swelling ratio of ≤1.5.

Paragraph 3. The synthetic hydrogel cell culture matrix of paragraph 1 or 2, having a stiffness of ≤150 Pa.

Paragraph 4. A synthetic hydrogel cell culture matrix for microfluidic organogenesis, comprising:
a branched polyalkylene glycol;
one or more integrin peptides; and
one or more extracellular matrix (ECM) binding peptides,
wherein the one or more integrin peptides and the one or more ECM binding peptides are bound to the branched polyalkylene glycol,
wherein the one or more integrin peptides comprise GFOGER (SEQ ID NO:1) and RGD (SEQ ID NO:3),
wherein the molar ratio of the one or more integrin peptides to the one or more ECM binding peptides is greater than 1:1, and
wherein the synthetic hydrogel cell culture matrix has a swelling ratio of ≤1.5 and/or a stiffness of ≤150 Pa.

Paragraph 5. The synthetic hydrogel cell culture matrix of paragraph 4, wherein the one or more integrin peptides further comprise YIGSR (SEQ ID NO:45).

Paragraph 6. The synthetic hydrogel cell culture matrix of any one of paragraphs 1-5,
wherein the one or more ECM binding peptides comprise NH₂-GCREISAFLGIPFAEPPMGPRRFLPPEPKKP(Am) (SEQ ID NO:5) and NH₂-GCRE-TLQPVYEYMVGV-COOH (SEQ ID NO:6).

Paragraph 7. The synthetic hydrogel cell culture matrix of any one of paragraphs 1-6, further comprising a dithiol crosslinking peptide bound to the branched polyalkylene glycol, optionally via a thioether sulfone bond.

Paragraph 8. The synthetic hydrogel cell culture matrix of paragraph 7, wherein the dithiol crosslinking peptide comprises (Ac)-GCRDLPRTGGPQGIWGQDRCG(Am) (SEQ ID NO:24) at the time of crosslinking.

Paragraph 9. The synthetic hydrogel cell culture matrix of any one of paragraphs 1-8, having a crosslinking ratio of ≥0.52.

Paragraph 10. The synthetic hydrogel cell culture matrix of any one of paragraphs 1-9, wherein the branched polyalkylene glycol is at a nominal concentration of ≤3 wt %.

Paragraph 11. The synthetic hydrogel cell culture matrix of any one of paragraphs 1-3 and 5-10, wherein the YIGSR (SEQ ID NO:45) is at a nominal concentration ranging from about 0.1 mM to about 1 mM.

Paragraph 12. The synthetic hydrogel cell culture matrix of any one of paragraphs 1-11, wherein the one or more integrin peptides and one or more ECM binding peptides are bound to the branched pollyalkylene glycol via a thioether sulfone bond.

Paragraph 13. A hydrogel precursor composition for synthetic hydrogel cell culture matrix, comprising:
a branched polyalkylene glycol;
one or more integrin peptides, and
one or more extracellular matrix (ECM) binding peptides; and
a carrier,
wherein the one or more integrin peptides and the one or more ECM binding peptides are bound to the branched polyalkylene glycol,
wherein the one or more integrin peptides comprise GFOGER (SEQ ID NO:1), RGD (SEQ ID NO:3), and YIGSR (SEQ ID NO:45), and
wherein the molar ratio of the one or more integrin peptides to the one or more ECM binding peptides is greater than 1:1.

Paragraph 14. The hydrogel precursor composition of paragraph 13, wherein the one or more ECM binding peptides comprise NH₂-GCREISAFLGIPFAEPPMGPRRFLPPEPKKP(Am) (SEQ ID NO:5) and NH₂-GCRE-TLQPVYEYMVGV-COOH (SEQ ID NO:6).

Paragraph 15. The hydrogel precursor composition of paragraph 13 or 14, wherein the branched polyalkylene glycol is at a nominal concentration of ≤3 wt %.

Paragraph 16. The hydrogel precursor composition of any one of paragraphs 13-15, wherein the YIGSR (SEQ ID NO:45) is at a nominal concentration ranging from about 0.1 mM to about 1 mM.

Paragraph 17. The hydrogel precursor composition of any one of paragraphs 13-16, wherein the one or more integrin peptides and one or more ECM binding peptides are bound to the branched pollyalkylene glycol via a thioether sulfone bond.

Paragraph 18. The hydrogel precursor composition of any one of paragraphs 13-17, wherein the carrier is a buffer.

Paragraph 19. A kit for synthetic hydrogel cell culture matrix comprising:
(a) the hydrogel precursor composition of any one of paragraphs 13-18;
(b) a dithiol crosslinking peptide; and
(c) optionally one or more types of cells.

Paragraph 20. The kit of paragraph 19, further comprising a microfluidic device.

Paragraph 21. A method for vascularized organogenesis comprising:
(i) incorporating into the hydrogel precursor composition of any one of paragraphs 13-18 (1) endothelial cells and (2) optionally stromal cells, macrophages, epithelial cells, and/or organoids; and
(ii) mixing the endothelial cells-incorporated hydrogel precursor composition with a dithiol crosslinking peptide to form a pre-gel solution.

Paragraph 22. The method of paragraph 21, further comprising (iii) loading the pre-gel solution into a microfluidic device.

Paragraph 23. The method of paragraph 21 or 22, further comprising, following step (ii) or (iii), (a) maintaining the pre-gel solution at a temperature for a time period sufficient to form a hydrogel.

Paragraph 24. The method of paragraph 23, further comprising, following step (a), (b) exposing the hydrogel to a cell culture media at a temperature for a time period sufficient to form a vascular tissue.

Paragraph 25. The method of paragraph 24, wherein in step (b), the cell culture media is added to a microfluidic channel at a flow rate ranging from about 0.1 μL/s to about 10 μL/s, from about 0.5 μL/s to about 5 μL/s, from about 0.5 μL/s to about 2 μL/s, such as about 1 μL/s; and/or with a pressure drop ranging from about 10 mm H₂O to about 50 mm H₂O, from about 15 mm H₂O to about 35 mm H₂O, or from about 20 mm H₂O to about 30 mm H₂O, such as about 25 mm H₂O.

Paragraph 26. The method of any one of paragraphs 22-25, further comprising, prior to step (iii), (c) immersing the microfluidic device in an estradiol solution at a temperature for a time period sufficient to pre-saturate the microfluidic device with estradiol; and (d) drying the pre-saturated microfluidic device.

Paragraph 27. The method of any one of paragraphs 21-26, wherein the dithiol crosslinking peptide comprises (Ac)-GCRDLPRTGGPQGIWGQDRCG(Am) (SEQ ID NO:24).

Paragraph 28. The method of any one of paragraphs 23-27, wherein the hydrogel has a swelling ratio of ≤1.5 and/or a stiffness of ≤150 Pa.

Paragraph 29. The method of any one of paragraphs 23-28, wherein the vascular tissue is a vasculature with open lumen or a vascular lesion.

Paragraph 30. The method of paragraph 29, wherein the vascular tissue is uterine bed vasculature or endometriosis lesion.

The present invention will be further understood by reference to the following non-limiting examples.

### EXAMPLES

### Example 1: Synthesis of Synthetic PEG-Crosslinked Peptide-Adhesion Peptide ECM Supports Human-Derived Endometrial Organoid Culture (Corresponds to Example 4 of PCT/US2020/044067)

The endometrium is a highly dynamic mucosal barrier tissue which sheds about 1 cm of glandular and luminal epithelia along with supporting stroma each month and then regenerates the tissue from remaining pools of stem and progenitor cells. Regeneration and growth are primarily fueled by estradiol, and tissue differentiation processes (i.e., decidualization) commences upon the onset of progesterone after ovulation. A study to include human endometrial organoids was conducted, as they exhibit many similarities to intestinal organoids when cultured in MATRIGEL®: a single layer of epithelium surrounding a lumen; strongly proliferative in a culture medium favoring stem cells; responsive to differentiation cues; robust to serial passage for 5+ months without changes in DNA; and robust recovery after a freeze/thaw cycle.

### Materials and Methods

Multi-arm polyethylene glycol (PEG) polymers of various molecular weights, small peptides, and inhibitors were identified as the components formulating the hybrid hydrogels. Specifically, 20 kDa 8-arm PEG, 40 kDa 8-arm PEG, or a combination of both were identified for forming PEG hydrogels. The PEG arms were functionalized with vinyl sulfone (VS) groups.

Integrin binding peptides derived from collagen type I (containing the sequence GFOGER (SEQ ID NO:1)) and fibronectin (containing the sequence PHSRN (SEQ ID NO:2)) were selected for cell adhesion. The peptide derived from collagen type I has high affinity for integrin α2β1, α1β1, α10β1, and α11β1. The peptide derived from fibronectin has high affinity for integrin αvβ3 and moderate affinity for integrin α5β1. The integrin binding peptides were synthesized to contain a free thiol group at the N-terminus. The collagen type I- and fibronectin-derived adhesion peptides can be used individually or in combination in the hydrogel formulation.

ECM binding peptides having affinity for secreted collagen and laminin (“BM binders”) (NH2-GCREISAFLGIPFAEPPMGPRRFLPPEPKKP(Am)) (SEQ ID NO:5), and fibronectin (“FN binders”) (NH2-GCRE-TLQPVYEYMVGV-COOH) (SEQ ID NO:6)), were selected. These extracellular protein binding peptides were designed to capture fibronectin produced by endometrial stromal fibroblasts throughout the cycle and both laminin and type IV collagen basement membrane proteins that are normally produced by the epithelium during all menstrual cycle stages and locally produced and accumulated in the pericellular region of hormonally responsive fibroblasts in the stroma (Cook, et al., Integrative Biology, 9:271-289 (2017)). The extracellular protein binding peptides were synthesized to contain a free thiol group at the N-terminus.

Peptides that are cleavable by a matrix-metalloproteinase (MMP) (Ac)GCRD-GPQGIAGQ-DRCG(Am) (SEQ ID NO:10) or Sortase A transpeptidase (Sortase) are identified as crosslinkers to form the final cross-linked hydrogels.

The PEG polymer arms were functionalized with vinyl sulfone groups (purchased from JenKem Technology, Beijing). The integrin-binding peptides and extracellular protein binding peptides were synthesized to contain a free thiol group at the N-terminus. The crosslinkers are dithiols with one thiol group at each terminus. The PEG hydrogels (3.5-10%, weight/volume) were assembled via sequential Michael-type addition reactions between the vinyl sulfone groups on the PEG polymer arms and the terminal thiol groups of the integrin binding peptides (0.25-3 mM) and extracellular protein binding peptides (0.25-0.5 mM) (FIG. 1). Cells and ROCK inhibitors were then mixed with the peptide-PEG polymer adducts. The peptide-PEG polymer adducts were crosslinked with crosslinkers (thiol of cross linker:VS, 0.35-1), such as dithiol-MMP cleavable peptides. The PEG hydrogels are fully assembled after 30 minutes at pH 7.4 at 37° C. The Michael type addition reaction occurs at physiological conditions thus cells are not under stress during cell-encapsulation). Endometrial organoids were established using published protocols and cultured in endometrial organoid medium (EnOM) (Boretto et al., Nat. Cell Biol. 21:1041-1051 (2019), Turco et al., Nat. Cell Biol. 19:568-577 (2017)).

PEG macromers and peptides. 8-arm PEG macromers with vinyl sulfone (VS) terminal groups (40,000 MW PEG-VS, “PEG-40” and 20,000 MW PEG-VS, “PEG-20”) were obtained from JenKem Technology (Beijing). All peptides were custom synthesized and purified by Boston Open Labs (Cambridge, MA), GenScript (Piscataway, NJ), or CPC Scientific (Sunnyvale, CA). Peptides sequences and their relevant features are included in the Table 3. “XL-MMP”, a crosslinker containing a matrix metalloproteinase (MMP)-sensitive substrate, “XL-MMP-SrtA” a crosslinker containing a MMP-sensitive substrate and a Sortase-sensitive recognition site (LPRTG), “XL-SrtA,” a crosslinker containing only the Sortase-sensitive site, “RGD,” a fibronectin (FN)-derived peptide containing the canonical RGD motif from the 10th FN type III domain, “PHSRN-K-RGD,” a FN-derived peptide containing the RGD motif and the PHSRN (SEQ ID NO:2) synergy site from the 9th FN type III repeat in a branched configuration; “GFOGER,” a collagen I-derived peptide presented as a triple helix (Wojtowicz et al., Biomaterials, 31:2574-2582 (2010), Carafoli et al., PLoS 25 ONE, 7(7):e42473 (2012)), “GFOGDR,” a collagen I-derived peptide with E→D point mutation that reduces integrin binding (Knight et al., J. Biol. Chem. 275:35-40 (2000)), “G11RGD,” an extended ligand of RGD with eleven Gly spacers, “CMPRGD,” an extended and clustered RGD-containing ligand using a triple helical collagen mimetic peptide (Hernandez-Gordillo et al., Biomaterials, 35:7363-7373 (2014)), “FN-binder,” a peptide with affinity for FN, and “BM-binder,” a peptide with affinity for collagen IV (C-IV) and laminin (LMN) (Johnson et al., Biochem. Biophys. Res. Commun., 319:448-455 (2004)). All peptides were reconstituted in acidic (pH 5.2) Milli-Q water (Millipore). The concentration of free thiols in all peptides was determined using Ellman's reagent (Sigma). Other peptides were as described in Zustiak and Leach, Biomacromolecules. 11:1348-1357 (2010); Kassis et al., Sci. Rep. 9:12479 (2019); and Schindelin et al, Nat. Methods. 9:676-682 (2012).

TABLE 3
Peptide sequences and relevant features.

| Name | Sequence | Relevant Feature |
|---|---|---|
| XL- | (Ac)-GCRD-VPMSMRGG- | a dithiol peptide containing a matrix |
| MMP | DRCG-(Am) | metalloproteinase (MMP)-sensitive |
| | (SEQ ID NO: 29) | substrate |
| XL- | (Ac)-GCRD-LPRTG- | a dithiol peptide containing a MMP- |
| MMP- | GPQGIAGQ-DRCG-(Am) | sensitive substrate and a Sortase-sensitive |
| SrtA | (SEQ ID NO: 30) | recognition site (LPRTG) |
| XL- | (Ac)-GCRD-LPRTG-DRCG- | a dithiol peptide containing only the |
| SrtA | (Am) (SEQ ID NO: 31) | Sortase-sensitive site (LPRTG) |
| RGD | (Ac)-GCRE-RGDSP-(Am) | a fibronectin (FN)-derived peptide |
| | (SEQ ID NO: 4) | containing the canonical RGD motif from |
| | | the 10th FN type III domain |
| PHSRN- | (Ac)-PHSRNGGGK-GGG- | a FN-derived peptide containing the RGD |
| K-RGD | ERCG-(Am)-GGRGDSPY- | motif and the PHSRN (SEQ ID NO: 2) |
| | (Am) (SEQ ID NO: 32) | synergy site from the 9th FN type III repeat |
| | | in a branched configuration, |
| GFOGER | (Ac)- | a collagen I-derived peptide presented as a |
| | GGYGGGPG(GPP)₅GFOGER | triple helix, |
| | (GPP)₅GPC-(Am) | |
| | (SEQ ID NO: 12) | |
| GFOGDR | (Ac)- | a collagen I-derived peptide with a point |
| | GGYGGGPG(GPP)₅GFOGDR | mutation E→D that reduces integrin |
| | (GPP)₅GPC-(Am) | binding |
| | (SEQ ID NO: 33) | |
| G11RGD | (Ac)-GCRE-GGGGGGGGGGG | an extended ligand of RGDS with eleven |
| | RGDSP-(Am) | Gly spacers |
| | (SEQ ID NO: 34) | |
| CMPRGD | (Ac)CGG(POG)₉RGDSP(Am) | an extended and clustered RGDS ligand |
| | (SEQ ID NO: 35) | using a triple helical collagen mimetic |
| | | peptide |
| FN- | (Am)-GCRE- | a peptide with affinity for FN |
| binder | TLQPVYEYMVGV-Ac) | |
| | (SEQ ID NO: 14) | |
| BM- | Ac-GCRE- | a peptide with affinity for collagen IV |
| binder | ISAFLGIPFAEPPMGPR | (C-IV) and laminin (LMN) |
| | RFLPPEPKKP-(Am) | |
| | (SEQ ID NO: 7) | |

Synthesis of hydrogel precursors. All synthetic matrices were assembled at 3 or 5% PEG (w/v) using 8-arm PEG-40 or 8-arm PEG-20. The mixed PEG-40/20 synthetic matrix was made mixing 1:1 (v/v, 2.5% 8-arm PEG-40 and 2.5% 8-arm PEG-20). All matrices were assembled stepwise via Michael-type addition reaction. First, the PEG-VS macromer (either PEG-40, PEG-20, or the PEG-40/20 mix) was diluted with 10×PBS-HEPES solution (1 mM, pH 7.8); second, the matrix-binder peptides were added to the PEG reaction mixture and incubated for 30 minutes at RT; third, the integrin binder peptides (RGD, PHSRN-K-RGD, GFOGER, G11RGD, CMPRGD or GFOGDR) were added to the reaction mixture and allowed to react for an additional 30 min at RT. This sequential reaction created a PEG-functionalized mixture “fPEG-VS” that was used to resuspend the cells prior to matrix gelation (see cell encapsulation below). The nominal concentration of the matrix-binders in all matrices was 0.25 mM each, whereas the integrin binder peptides were at 1.5 mM (unless otherwise noted in the figure legends). For most experiments the fPEG-VS solution was crosslinked at 50% crosslinking density (unless otherwise noted).

Cell culture. Organoids were passaged every four days for human intestinal organoids, eight days for human endometrial organoids and three days for mouse organoids. For passaging, organoids were incubated in Cell Recovery Solution (CRS, Gibco) for 1 h at 4° C., followed by trypsin treatment to generate single cells. The cell suspension was inspected under the microscope to ensure the presence of dispersed single cells and if needed, filtered through a 30 μm cell strainer to remove cell clumps. Single cells were counted using a hemocytometer and seeded in GFR MATRIGEL® (25 μL droplet) at a density of 1,000 cells/μL for human and 500 cells/μL for mouse organoids. After MATRIGEL® polymerization, 600 μL of EM or EnOM was layered on top. Media was changed every two days.

Organoid differentiation. Organoids in both MATRIGEL® and the synthetic matrix were cultured in EM for six days (human) or four days (mouse) before switching to differentiation medium. Human and mouse organoids in MATRIGEL® were differentiated as reported by Kasendra et al., Sci. Rep. 8:2871 (2018), Miyoshi et al., Nat. Protoc. 8:2471-82 (2013). Intestinal organoids in the synthetic matrix were differentiated using L-WRN conditioned medium (25% L-WRN) diluted in Adv DMEM/F12, HEPES (1 μM, Gibco), Pen/Strep (100 U/mL, 100 mg/mL, Invitrogen), N2 supplement (1×, Gibco), B27 supplement (1×, Gibco), human [Leu15]-gastrin I (10 nM, BACHEM), N-acetyl cysteine (1 mM, SIGMA) and Y-27632 (5 μM, TOCRIS). Mouse organoids in the synthetic ECM were differentiated as in MATRIGEL®.

Organoids were established derived from endometrial biopsies obtained from the stratum functionalis of human donors. In comparison to intestine, the endometrial stem cell compartment is relatively poorly defined, and the tissue exhibits dramatic steroid hormone responses. One study using in situ hybridization for LGR-5 RNA showed that the endometrium potentially has stem/progenitor pools in the functionalis region of the endometrium, from which the donor tissue was harvested, throughout the menstrual cycle. The geographical location of putative stem/progenitor cell compartments aids selection of synthetic ECM components in light of reported integrin expression profiles in the endometrium. Integrin alpha 2 beta-1 is expressed throughout all phases of the menstrual cycle and is strongly expressed in luminal and glandular epithelia, thus motivating the inclusion of the GFOGER peptide. The RGD-binding vitronectin receptor, integrin alpha-beta3, has an unusual cyclical expression profile. In healthy women, it is expressed in mid-secretory phase throughout implantation and may play a role in embryo implantation. Its expression in the endometrial organoids would not be expected due to control over sex hormone concentrations in the in vitro model. While it is well-established that the endometrial stromal cells express the canonical fibronectin-binding receptor, integrin alpha-5-beta1, its expression in the epithelia is controversial. Integrin β6, which functions as a LMN receptor when paired with al or a4, is expressed in basolateral fashion throughout the endometrium in proliferative and secretory phases. A screen of ECMs containing PEG-20-GFOGER, PEG-20-PHSRN-K-RGD, PEG-20-RGD, PEG-20-G11RGD, and PEG-20-CMPRGD yielded a formulation suitable for culture of endometrial organoids.

Single cells obtained from dissociation of endometrial organoids were embedded in the synthetic ECMs, where they proliferated and formed lumenized cystic organoids with thick columnar cells. Among the formulations tested, the best synthetic ECM formulation for supporting robust growth of polarized endometrial organoids was identical to the best synthetic ECM for enteroids). Similar to intestinal enteroids, endometrial organoids in MATRIGEL® appeared larger and more abundant than organoids in the synthetic matrices eight days after encapsulation. In the PEG-20-GFOGER, the organoid median diameter was 85 μm, compared to 58 μm for the PEG-20-PHSRN-K-RGD. In MATRIGEL® the median diameter was 91 μm. Approximately 38% of all endometrial organoids counted in the PEG-20-GFOGER matrix reached sizes above 90 microns, compared to 21% in the PEG-20-PHSRN-K-RGD. Both synthetic ECMs, however, were lower than MATRIGEL® in which 46% of the organoid population reached diameters above 90 microns. Finally, endometrial organoids, in the PEG-20-GFOGER matrix, showed proliferative cells (EdU), epithelial EpCAM, apical actin, and basal LMN deposition similar to those grown in MATRIGEL®. Immunostaining analysis of organoids in the PEG-20-GFOGER matrix made at 1.5 mM (0.5 mM GFOGERth), 0.25 mM BM-binder, 0.25 FN-binder and 50% XL-MMP-SrtA crosslinker, showed proliferative cells (EdU), apical markers (actin), epithelial marker (EpCAM), and basolateral marker (LMN). Collectively, these data showed that a defined intestinal formulation of a hybrid hydrogel can be applicable to additional epithelial-rich tissues.

Hydrogel samples were prepared for cell culture and characterization using the following procedure: first, 20 kDa, 8-arm end-functionalized PEG-vinyl sulfone (PEG-VS) was combined with a mixture of integrin-adhesive and secreted protein-sequestering pendant (monofunctional) peptides in a buffered solution at a pH of ~8-8.4 for 30 min at room temperature. This mixture was prepared with approximately 4-fold excess of vinyl sulfone groups relative to pendant peptide thiol groups, providing free alkenes for later hydrogel crosslinking. The PEG-peptide was then diluted in neutral buffer, and that mixture was used to resuspend human endometrial epithelial cells (EECs). Human endometrial epithelial cells were cultured over the course of 7-14 days and the emergence of EEOs as well as their morphology and polarization were assessed via brightfield and confocal microscopy, respectively.

A fully synthetic matrix is described that supports primary, human tissue-derived, enteroids from the upper and lower intestinal track and primary, human tissue-derived, endometrial epithelial organoids. The local biophysical presentation of an α2β1 integrin-binding peptide (GFOGER) in the presence of peptides that sequester cell-produce ECM appear as the dominant variables governing epithelial cell proliferation and organoid formation in PEG-based synthetic ECMs. While the synthetic matrix does not match MATRIGEL® benchmarks by some metrics (rate of human organoid emergence and overall proliferation over 6-8 days), it is superior to MATRIGEL® by other metrics, in applications that require (i) a rigorously defined microenvironment to interrogate basic human stem cell biology; (ii) long-term growth of human organoids beyond 6 days; and (iii) in vivo human applications where MATRIGEL® would violate GMP protocols.

The synthetic ECM offers additional benefits such as on-demand dissolution to recover intact organoids and other co-cultured cells for in depth omics analysis and downstream cell-secreted metabolites quantification. This feature is of great utility to uncover complex and dynamic cell-cell communications in emerging stromal-epithelial co-culture systems.

Primary human endometrial epithelial organoids were maintained in culture in this material up to 2 weeks. The cells exhibit strong and clear polarization, which suggests that their epithelial phenotype is preserved.

### Example 2: Characterization of Gel Mechanical Properties and Comparison for Use in Endometrial Epithelial Organoid Culture

Synthetic polymer hydrogels are a fundamental tool in tissue engineering, offering increased user tunability and reproducibility over natural polymer hydrogels. However, the development of a synthetic hydrogel which can support the co-culture of multiple different primary human cell types remains challenging. Hyaluronic acid (HA) gels are attractive as chemically modifiable and endogenously bioactive platforms. Additionally, HA is a component of the human endometrial extracellular matrix, and its metabolism by endometrial cells is altered in the differentiation process known as decidualization in vitro. Here, a hybrid hydrogel was developed using a combination of bioactive peptide-functionalized multi-arm poly(ethylene glycol) (PEG) and chemically modified HA. The fitness of this gel as a 3D culture substrate for primary human endometrial epithelial organoids (EEOs) was evaluated.

### Materials and Methods

Materials. 8-arm, 20 kDa PEG macromers (“PEG-VS”) synthesized with a tripentaerythritol core and functionalized with vinyl sulfone terminal groups (molar degree of substitution <90%) were obtained from JenKem Technology (Beijing) and used as described without further modification. Thiol-functionalized (“thiolated”) hyaluronic acid (HA-SH) with a molecular weight of approximately 300 kDa and molar degree of substitution of approximately 20-30% under the product name “GLYCOSIL” was obtained from Advanced Biomatrix and was used as described without further modification. HA-SH was thiolated at the carboxylic acid residues of the D-glucuronic acid portion of the repeating units (“monomers”) of the polymer, consisting of one D-glucuronic acid group and one N-acetyl-D-glucosamine group. The reported molar degree of substitution is the average fraction of monomers which have been functionalized with a thiol group at that carboxylic acid residue out of the total number of monomers in the batch of polymer.

HA-SH was reconstituted at 1% w/v in sterile phosphate buffered saline (PBS), pH 7.4 and stored in aliquots at −20° C. until use, without repeated freeze-thaw cycles. When stored at −20° C., the maximum shelf life of the frozen stock solutions was found to be approximately 2 months. A concentrated buffer solution of 10×Dulbecco's phosphate buffered saline and 1 M 4-(2-hydroxyethyl)-1-piperazineethanesulfonic acid (HEPES) was prepared by dissolving one packet of powdered Dulbecco's PBS (without Ca2+/Mg2+, sufficient for 1 L of buffer at 1×) (Gibco, REF #21600-051) in 100 mL 1M 4-(2-hydroxyethyl)-1-piperazineethanesulfonic acid (HEPES) (Gibco, REF #15630-080), pH 7.8-8.2. The pH of the concentrated HEPES/PBS solution (“HEPES/PBS”) was adjusted to 8.0-8.2 using 2 N sodium hydroxide in MilliQ distilled water, then sterile-filtered and stored in 1 mL aliquots at 4 C, covered to protect from light. SIGMACOTE® (a chlorinated organopolysiloxane, Sigma-Aldrich) was purchased and used according to the manufacturer's specifications to treat glass surfaces to make them more hydrophobic.

Peptides: All peptides were custom synthesized and purified by GenScript (Piscataway, NJ) or CPC Scientific (Sunnyvale, CA). Peptides sequences, their function in the gel, and the abbreviations by which they are referenced are included in Table 4. Importantly, the inclusion of cysteine residues in the peptide sequences enabled addition of the molecule to the gel via the reactivity of the thiol group on the cysteine side chain with the alkene vinyl sulfone on the PEG macromers.

For the purposes of gel assembly, the thiol group on the peptide is the reactive functional group. Peptides with one thiol group (i.e., one cysteine residue) are referred to as “monofunctional” or “pendant” peptides, and peptides with two thiol groups (i.e., two cysteine residues) are referred to as “bifunctional” or “crosslinking” peptides. Note that all peptides were modified post-synthesis by the manufacturer with N-terminal acetylation (“Ac”) and C-terminal amidation (“Am”). In the peptide sequences non-canonical amino acid hydroxyproline is represented by the letter O. Peptides used include:

“LW,” a peptide with one cysteine residue at each end to facilitate crosslinking of PEG-VS macromers, containing a MMP-labile domain GPQGIWGQ (SEQ ID NO:43) and a Sortase-labile domain LPRTG (SEQ ID NO:27);

“PHSRN-K-RGD,” a fibronectin-derived peptide containing the RGD motif and the PHSRN (SEQ ID NO:2) synergy site from the 9th fibronectin type III repeat in a branched configuration;

“GFOGER,” a collagen I-derived peptide presented as a triple helix (Wojtowicz et al., Biomaterials, 31:2574-2582 (2010), Carafoli et al., PLoS ONE, 7(7):e42473 (2012));

“FN-binder,” a peptide with non-covalent binding affinity for fibronectin; and

“BM-binder,” a peptide with non-covalent binding affinity for collagen IV and laminin (Johnson et al., Biochem. Biophys. Res. Commun., 319:448-455 (2004)).

The exemplary peptides described above may have the sequences or contain the sequences as specified in Table 4 below. For example, a LW peptide suitable for use in the hydrogel may be a peptide of SEQ ID NO:42 or a peptide that contains the sequence of SEQ ID NO:42.

All peptides were reconstituted in ultrapure Water (Invitrogen), with pH adjusted using 2 N sodium hydroxide solution in MilliQ distilled water to facilitate solubility when needed.

The concentration of peptide in the reconstituted stock solution were evaluated using 205 nm absorbance, with the exception of the GFOGER peptide. Due to light scattering of the triple-helical trimer in solution, the 205 nm absorbance of the GFOGER peptide was artificially elevated, so the concentration of the stock solution was measured by proxy using the concentration of free thiols, determined using Ellman's reagent (Sigma). Other peptides are as described in Zustiak and Leach, Biomacromolecules. 11:1348-1357 (2010); Kassis et al., Sci. Rep. 9:12479 (2019); and Schindelin et al, Nat. Methods. 9:676-682 (2012).

TABLE 4
Peptides included in gel formulations

| | | Sequence | |
|---|---|---|---|
| Abbreviation | Function | (1 letter AA code) | Manufacturer |
| PHSRN-K- | Integrin-binding, cell | (Ac)-PHSRN-GGGKGGGER | CPC |
| RGD | adhesive domain RGD | CG(Ac)-GGRGDSPY-(Am) | Scientific |
| | with synergistic | (SEQ ID NO: 36) | |
| | domain PHSRN | | |
| GFOGER | Integrin-binding, cell- | (Ac)-GGYGGGPGGPPGPP | GenScript |
| | adhesive domain | GPPGPPGPPGFOGERGPPG | |
| | GFOGER flanked by | PPGPPGPPGPPGPC-(Am) | |
| | repeating GPP | (SEQ ID NO: 37) | |
| | domains which self- | | |
| | assemble in aqueous | | |
| | solution into a triple- | | |
| | helix trimer | | |
| BM-binder | ECM-protein binding | (Ac)-GCREISAFLGIPFAE | CPC |
| | domain with a high | PPMGPRRFLPPEPKKP-(Am) | Scientific |
| | affinity for collagen | (SEQ ID NO: 7) | |
| | IV and laminins | | |
| FN-binder | ECM-protein binding | (Ac)-KKGCREPLQPVYE | CPC |
| | domain with high | YMVGV-(Am) (SEQ ID | Scientific |
| | affinity for fibronectin | NO: 41) | |
| LW | Crosslinking peptide | (Ac)-GCRDLPRTGGPQGI | GenScript |
| | for use in PEG-peptide | WGQDRCG-(Am) (SEQ ID | |
| | gels, degradable by | NO: 42) | |
| | cell-secreted MMP as | | |
| | well as by exogenous | | |
| | Sortase A | | |

Preparation of Gel Samples for Characterization. Acellular gel samples were prepared to identify HA-crosslinked (“PEG-HA”) and MMP-degradable peptide-crosslinked (“PEG-peptide”) formulations with comparable mechanical properties. Notably, both PEG-peptide and PEG-HA gel formulations used identical concentrations of peptides PHSRN-K-RGD, GFOGER, BM-binder, and FN-binder. Two gel formulations, one “soft” and one “stiff” were selected and used for characterization and subsequent cell culture experiments. The detailed composition of these two PEG-peptide formulations are included in Table 5. PEG-peptide gel samples with an initial (pre-polymerization and pre-swelling) volume of 50 μL each were prepared in the manner reported by Hernandez-Gordillo et al. https://doi.org/10.1016/j.biomaterials.2020.120125 with modification. Briefly, PEG-VS was diluted from a 10% w/v stock solution with HEPES/PBS and ultrapure Water (Invitrogen) in a microtube and vortexed to mix. Then, stock solutions of pendant peptides PHSRN-K-RGD, GFOGER, BM-binder, and FN-binder were added to the PEG-VS/buffer mixture and vortexed, then allowed to sit at room temperature for 30 min to allow the PEG-VS macromers to become functionalized with the pendant peptides via thiol-ene Michael addition reaction to form a PEG-peptide macromer. Then, the LW peptide was added to the PEG-peptide macromer solution to the final concentration, the solution was mixed via vortex for 10-15 s, and 50 μL of the pre-gel solution was immediately added to an 8 mm diameter, 0.5 mm thick round mold made from silicone sheet (McMaster Carr) pressed onto a SIGMACOTE®-treated glass microscopy slide. The pre-gel solution in the molds was allowed to polymerize into a gel for 30 min in a humidified chamber at 37° C. and 5% CO₂. After polymerization, the gel samples were removed from the molds and placed in PBS, pH 7.4, to swell.

TABLE 5
Detailed concentrations of each component in PEG-peptide gel formulations

| | [PEG- | | | [PHSRN- | | [BM- | [FN- | |
|---|---|---|---|---|---|---|---|---|
| | VS] (% | [HEPES] | | K-RGD] | [GFOGER] | binder] | binder] | [LW] |
| Formulation | w/v) | (mM) | [PBS] | (mM) | (mM) | (mM) | (mM) | (mM) |
| Soft | 2.5 | 100 | 1X | 1.5 | 1.5 | 0.5 | 0.5 | 2.60 |
| Stiff | 5.0 | 100 | 1X | 1.5 | 1.5 | 0.5 | 0.5 | 5.60 |

Three PEG-HA gel formulations were used for characterization with varying concentrations of HA-SH, the details of which are included in Table 5. For subsequent cell culture experiments, the 0.25% w/v HA-SH formulation was selected as the “soft” PEG-HA formulation and the 0.38% w/v HA-SH formulation was selected as the “stiff” PEG-HA 25 formulation. Preparation of PEG-HA gel samples is summarized in FIG. 1, and was performed as follows: First, HA-SH stock solution was thawed on a heat block at 37° C. for at least 1 h but no more than 2 h before addition to the pre-gel solution. Meanwhile, PEG-VS in a 20% w/v stock solution in ultrapure water was added to a microtube with HEPES/PBS, the pendant peptide stock solutions, and ultrapure water added to a volume sufficient to dilute all components to double the concentration in the final gel (Table 6). As the concentration of PEG-VS, HEPES/PBS, and the monofunctional pendant peptides are the same in all three PEG-HA formulations, the solution consisted of 5% w/v PEG-VS, 200 mM HEPES/2×PBS, 3.0 mM PHSRN-K-RGD, 3.0 mM GFOGER, 1.0 mM BM-binder, and 1.0 mM FN-binder. This mixture was vortexed to mix and allowed to react at room temperature for 30 min to form the pendant peptide-functionalized PEG-peptide macromer. After this, the HA-SH stock solution was diluted in PBS to double the final concentration in the gel (e.g., 0.5% w/v for the formulation with a final concentration of 0.25% w/v in the gel) and was vortexed 20-30 s to mix.

The PEG-peptide macromer solution in HEPES/PBS was then added 1:1 by volume to the diluted HA-SH solution to form the pre-gel solution. The pre-gel solution was immediately vortexed 10-15 s to mix and 50 uL was added to round molds identical to those used for preparing the PEG-peptide gel samples. The PEG-HA gel samples in molds were allowed to polymerized for 30 min in a humidified chamber at 37 C and 5% CO₂. The gels were then removed from the molds and placed in PBS to swell.

TABLE 6
Detailed concentrations of each component in PEG-HA gel formulations

| | | | | [PHSRN- | | [BM- | [FN- | [HA- |
|---|---|---|---|---|---|---|---|---|
| | [PEG-VS] | [HEPES] | | K-RGD] | [GFOGER] | binder] | binder] | SH] (% |
| Formulation | (% w/v) | (mM) | [PBS] | (mM) | (mM) | (mM) | (mM) | w/v) |
| 1 | 2.5 | 100 | 1X | 1.5 | 1.5 | 0.5 | 0.5 | 0.25 |
| 2 | 2.5 | 100 | 1X | 1.5 | 1.5 | 0.5 | 0.5 | 0.38 |
| 3 | 2.5 | 100 | 1X | 1.5 | 1.5 | 0.5 | 0.5 | 0.50 |

Volumetric Swelling Characterization. Post-polymerization, gel samples were allowed to swell to equilibrium (no net change in sample mass/volume) in divalent cation-free Phosphate Buffered Saline (PBS), pH 7.4 for 18-24 h at 37° C. and 5% CO₂, to approximate cell culture conditions. To assess volumetric swelling, the samples were removed from the PBS, excess moisture on the surface of the gel was removed, and the sample was moved to a glass microscopy slide surface-treated with SIGMACOTE® to prevent sticking and damage to the gel sample. The diameter and height in mm of the swollen gel sample was measured and used to calculate the volume in μL of the swollen gel at equilibrium (i.e., maximally swollen). To calculate the volumetric swelling factor (VSF), the volume (uL) of the equilibrium-swollen gel sample was divided by the initial volume of the sample (50 uL).

Characterization of Gel Mechanical Properties. The mechanical properties of the gel were assessed via shear oscillatory rheology using an Anton Paar MCR 302 rheometer with an 8 mm parallel plate measuring geometry and a Peltier plate with an air-controlled temperature regulation system. Equilibrium-swollen gel samples were placed on the Peltier plate sample stage, pre-warmed to 37° C., and the measuring geometry was lowered to 70-80% of the height of the equilibrium swollen sample. This compression of the sample prevented sample slippage during measurement. The sample was subject to 180 s time sweep at a frequency of 1 Hz and strain of 5% at 37° C. The storage and loss moduli were averaged over the time sweep for each sample. Those values for each sample were averaged to yield the average for the formulation (n=3), reported with the standard deviation. Frequency and amplitude sweeps were performed to verify that the testing parameters were within the linear viscoelastic regime (not reported).

Organoid culture. Endometrial epithelial cells (EEC) were isolated from patient biopsy samples and as described by Gnecco et al. Med. 2023 Aug. 11; 4(8):554-579.e9. doi: 10.1016/j.medj.2023.07.004. PMID: 37572651. After isolation, EECs were expanded as organoids in growth factor-reduced MATRIGEL® using methods detailed by Gnecco et al.

Organoid expansion in PEG-peptide and PEG-HA gels. After organoids were visible and had attained a maximum diameter of approximately 250 μm, EEOs were extracted from MATRIGEL® and dissociated into a single-cell suspension using cold (4° C.) Cell Recovery Solution (CRS, Gibco) for approximately 30 min. That cell suspension was centrifuged at 350 rcf for 5 min at 4° C. to pellet the cells, then the supernatant was aspirated and the cells were further treated with TrypLE Express supplemented with DNAse to dissociate any remaining clusters of cells completely into a homogenous single-cell suspension.

The EECs in suspension were counted, then divided into aliquots of ~500 cells/uL of gel in microtubes and centrifuged to pellet the cells. The cell pellets were kept on ice until use.

To encapsulate the EECs in soft and stiff PEG-peptide hydrogels for encapsulation into EEOs, the same general method was used for the two formulations, detailed in Table 6. First, PEG-VS, HEPES/PBS, PHSRN-K-RGD, GFOGER, BM-binder, and FN-binder stock solutions were added to a microtube and vortexed to mix. The mixture was allowed to react at room temperature for 30 min to form the PEG-peptide macromer. After 30 min, the PEG-peptide macromer solution was moved to ice until further use. The supernatant was then carefully and completely removed from one EEC pellet. The cells were resuspended in the PEG-peptide macromer solution. The LW stock solution was added to the mixture and pipetted up and down repeatedly to mix and form the pre-gel mixture (“pre-gel mixture”) referring to a mixture that includes all of the components necessary to form the final hydrogel sample before the components have polymerized/reacted to form the gel. “Mixture” includes cells and/or components that are not soluble in water. “Pre-gel solution” contains all the components necessary to form the final hydrogel but has not polymerized to form the gel, and all components are completely dissolved in water.) 3 μL droplets of pre-gel mixture were deposited per well in a 96-well, non-tissue culture-treated polystyrene plate. The well plate was moved to a humidified incubator at 37° C. and 5% CO₂ for 30 min to polymerize. 100 μL of EEO expansion media was then added to each gel sample and then the well plate was returned to the incubator.

To encapsulate the EECs in soft and stiff PEG-HA hydrogels for encapsulation into EEOs, the same general method was used for the two formulations. The “soft” PEG-HA gel formulation is listed in Table 6 as formulation 1: 2.5% w/v PEG-VS, 100 mM HEPES/1×PBS, 1.5 mM PHSRN-K-RGD, 1.5 mM GFOGER, 0.5 mM BM-binder, 0.5 mM FN-binder, and 0.25% w/v HA-SH. The “stiff” PEG-HA gel formulation is listed in Table 6 as formulation 2: 2.5% w/v PEG-VS, 100 mM HEPES/1×PBS, 1.5 mM PHSRN-K-RGD, 1.5 mM GFOGER, 0.5 mM BM-binder, 0.5 mM FN-binder, and 0.38% w/v HA-SH. First, the reaction mixture for a concentrated peptide-functionalized PEG macromer was made. 5% w/v 20 PEG-VS, 200 mM HEPES/2×PBS, 3.0 mM PHSRN-K-RGD, 3.0 mM GFOGER, 1.0 mM BM-binder, and 1.0 mM FN-binder were combined in a microtube and vortexed to mix. The mixture was allowed to react at room temperature for 30 min to form the PEG-peptide macromer. After 30 min, the concentrated PEG-peptide macromer solution was moved to ice until further use. The supernatant was then carefully and completely removed from one EEC pellet. The cells were resuspended in sufficient sterile PBS to achieve the desired final volume of the mixture after adding the PEG-peptide macromer solution and the HA-SH stock solution. The concentrated PEG-peptide macromer solution was then added to the cell suspension so that the PEG-peptide macromer solution will be diluted 2-fold in the final pre-gel mixture. Pipette the mixture up and down repeatedly to mix. The HA-SH stock solution was added to the mixture with the cells and pipetted up and down repeatedly to form the pre-gel mixture. Immediately after, 3 uL droplets of pre-gel mixture were deposited per well in a 96-well, non-tissue culture-treated polystyrene plate. The well plate was moved to a humidified incubator at 37° C. and 5% CO₂ for 30 min to polymerize. 100 μL of EEO expansion media was then added to each gel sample and then the well plate was returned to the incubator.

All samples were kept in culture for 14 days, with media changes every 3 days. Images of the samples were taken every day via brightfield microscopy to monitor EEO expansion.

EEO staining for polarization. After 14 days, gel samples were fixed in 4% paraformaldehyde and stained with DAPI and rhodamine phalloidin to identify cell nuclei and f-actin, respectively.

### Results

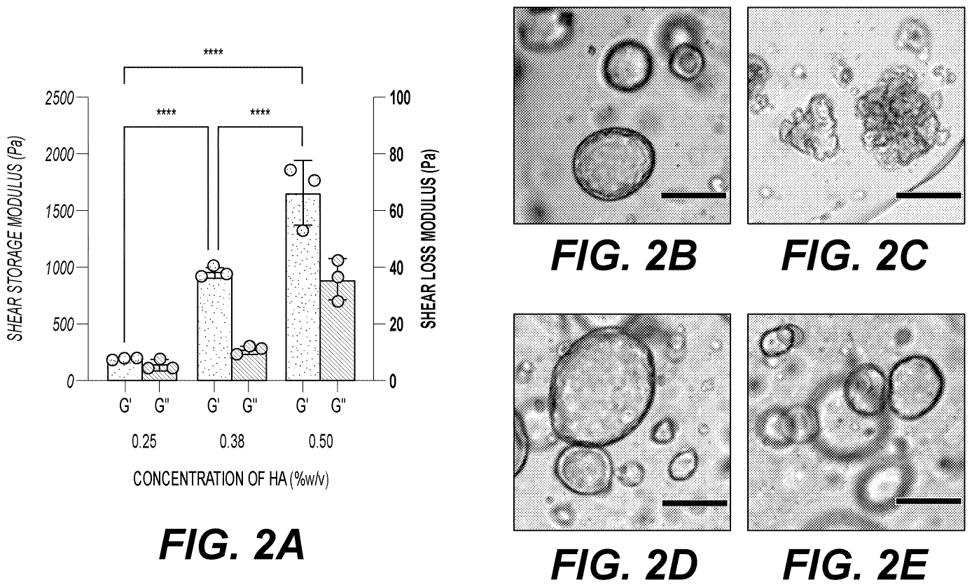

FIG. 2A is a graph of the gel shear storage modulus, G′ (Pa), and shear loss modulus, G″ (Pa) of acellular gel samples, showing that both G′ and G″ increase with increasing concentrations of HA-SH in the gel, from 0.25% w/v, 0.38% w/v, to 0.5% w/v. FIG. 2B-2E are micrographs of the cells in culture, showing morphology as a function of HA concentration. Though both G′ and G″ increase with increasing HA-SH content, the ratio of G″/G′, also expressed as the loss factor, tan(6), remains relatively constant between the formulations, indicating a similar degree of viscoelasticity between the formulations.

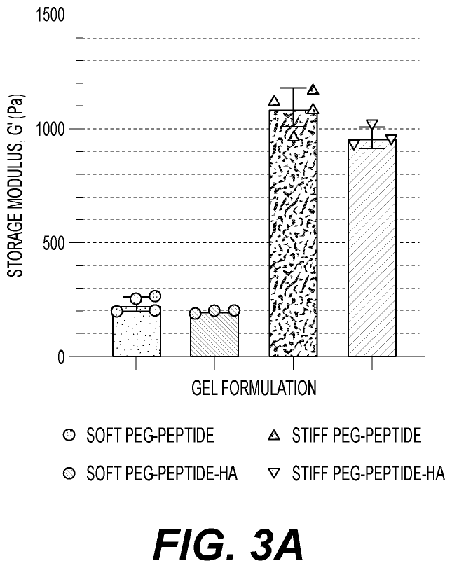

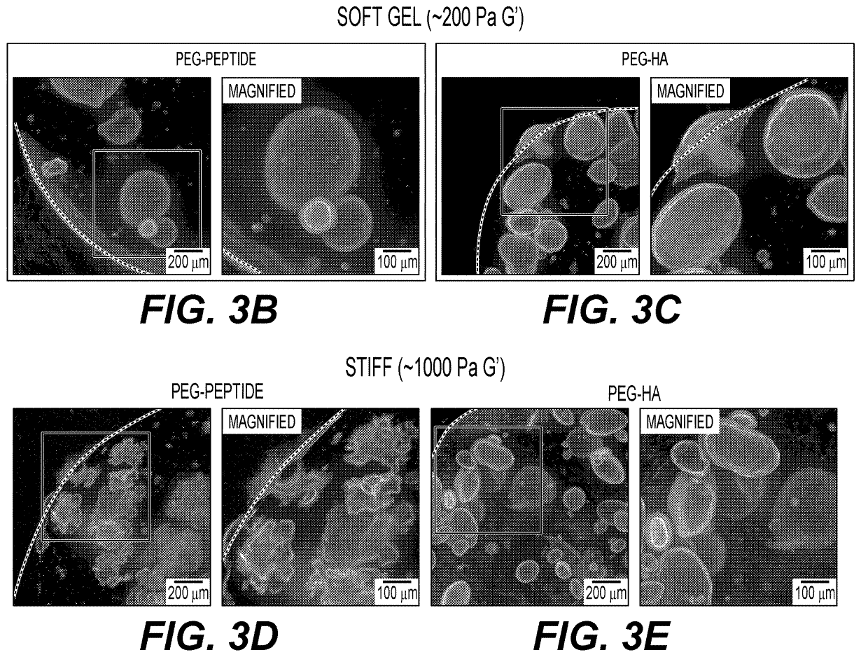

FIG. 3A shows the stiffness in shear storage modulus, G′ (Pa), of the soft PEG-peptide (Table 5, “Soft”) and PEG-HA (Table 6, “Formulation 1”, 0.25% w/v HA-SH) gel formulations and stiff PEG-peptide (Table 5, “Stiff”) and PEG-HA (Table 6, “Formulation 2”, 0.38% w/v HA-SH) gel formulations selected for comparison. Both peptide-crosslinked and HA-crosslinked PEG gels supported the expansion of human EEOs over the course of 14 days in both soft (~200 Pa G′) and stiff (~1000 Pa G′) formulations. FIG. 3B-E are representative fluorescent micrographs of the EEOs expanded for 14 days in culture and stained for f-actin (green) and cell nuclei (blue) in each of the four formulations: Soft PEG-peptide (FIG. 3B), Soft PEG-HA (FIG. 3C), Stiff PEG-peptide (FIG. 3D), and Stiff PEG-HA (FIG. 3E).

Remarkably, the soft and stiff PEG-peptide-HA conditions both displayed similar spherical and oblong organoid morphology, with luminal localization of f-actin staining indicative of characteristic epithelial polarization. In contrast, EEOs cultured in stiff (~1000 Pa G′) PEG-peptide gels without HA exhibited a disorganized morphology.

Analysis of f-actin polarization images at day 14 showed that the F-actin was polarized at the apical side in both the soft and stiff PEG-peptide gels as well as the soft and stiff PEG-HA gels.

However, PEG-peptide gels and PEG-HA exhibited differences in adhesion ligand distribution. PEG-peptide hydrogels show a homogenous distribution of FL-PHSRN-K-RGD; PEG-peptide+HA hydrogels show a micron-scale heterogeneity of FL-PHSRN-K-RGD distribution.

Cells were treated over a period of nine days for the effect on morphology of hormones. The results showed that gel selection (PEG-peptide or PEG-HA) had more influence on cell morphology than hormone. ESCs exhibit greater spreading in PEG-peptide hydrogels after nine days in culture, as compared to ESCs in PEG-HA. Analysis of f-actin polarization images at day 14 showed that the F-actin was polarized at the apical side in both the soft and stiff PEG-peptide gels as well as the soft and stiff PEG-HA gels. This is characteristic of endometrial epithelial cells, which exhibit polarization not only in their eutopic site in the endometrium, but also in ectopic lesions in endometriosis and adenomyosis.

Materials. 20 kDa, 8-arm poly(ethylene glycol)-vinyl sulfone (PEG-VS), HEPES and PBS buffers components, and synthetic peptides were obtained as described in EXAMPLE 2. Thiolated 10 kDa (“HA-10”, 30% molar degree of substitution), 50 kDa (“HA-50”, 10% molar degree of substitution), and 1500 kDa (“HA-1500”, 5% molar degree of substitution) HA was manufactured by and purchased from Creative PEGWorks. Stock solutions of HEPES/PBS buffer, PEG-VS, and peptide components were prepared as detailed in EXAMPLE 2. SIGMACOTE® (a chlorinated organopolysiloxane, Sigma-Aldrich) was purchased and used according to the manufacturer's specifications to treat glass surfaces to make them more hydrophobic.

HA-SH was reconstituted at 2% w/v for HA-10 and HA-1500 and at 2.5% w/v for HA-50 in sterile phosphate buffered saline (PBS), pH 7.4 and stored in aliquots at −20° C. until use, without repeated freeze-thaw cycles. When stored at −20° C., the maximum shelf life of the frozen stock solutions was found to be approximately 2 months.

Preparation of Gel Samples for Characterization. Acellular gel samples were prepared to evaluate the mechanical properties and swelling of formulations with varied molecular weights and concentrations of HA. The detailed composition of formulations tested is given in Table 5 and 6. Hydrogel samples with an initial (pre-polymerization and pre-swelling) volume of 50 μL each were prepared in the manner reported in EXAMPLE 2 above. First, a 2× concentrated solution of PEG-binder adduct was prepared. PEG-VS was diluted from a 20% w/v stock solution to 5% w/v with 200 mM HEPES/2×PBS and ultrapure Water (Invitrogen) in a microtube and vortexed to mix. Then, stock solutions of peptide binders were added to the following concentrations: 3.0 mM PHSRN-K-RGD, 3.0 mM GFOGER, 1.0 BM-binder, and 1.0 FN-binder. The mixture was allowed to sit at room temperature for 30 min to allow the PEG-VS macromers to become functionalized with the pendant peptides via thiol-ene Michael addition reaction to form a PEG-binder adduct. After this, the HA-SH stock solution was diluted in PBS to double the final concentration in the gel, if needed (e.g., 0.5% w/v for the formulation with a final concentration of 0.25% w/v in the gel) and was vortexed 20-30 s to mix.

The PEG-binder adduct solution in HEPES/PBS was then added 1:1 by volume to the diluted HA-SH solution to form the pre-gel solution. The pre-gel solution was immediately vortexed 10-15 s to mix and 50 uL was added to round molds identical to those used for preparing the PEG-peptide gel samples. The hydrogel samples in molds were allowed to polymerize for 30 min in a humidified chamber at 37 C and 5% CO₂. The gels were then removed from the molds and placed in PBS to swell.

### Example 3: Modifications to Include Alternative Molecular Weights of HA

Hyaluronic acid (HA) notably has physiochemical and biological properties which change with its molecular weight. Hydrogel formulation optimization was performed to identify what gel compositions could be utilized with higher and lower molecular weight HA compared to the preferred formulation (~300 kDa).

### Materials and Methods

Materials. 20 kDa, 8-arm poly(ethylene glycol)-vinyl sulfone (PEG-VS), HEPES and PBS buffers components, and synthetic peptides were obtained as described in EXAMPLE 2. Thiolated 10 kDa (“HA-10”, 30% molar degree of substitution), 50 kDa (“HA-50”, 10% molar degree of substitution), and 1500 kDa (“HA-1500”, 5% molar degree of substitution) HA was manufactured by and purchased from Creative PEGWorks. Stock solutions of HEPES/PBS buffer, PEG-VS, and peptide components were prepared as detailed in EXAMPLE 2. SIGMACOTE® (a chlorinated organopolysiloxane, Sigma-Aldrich) was purchased and used according to the manufacturer's specifications to treat glass surfaces to make them more hydrophobic.

HA-SH was reconstituted at 2% w/v for HA-10 and HA-1500 and at 2.5% w/v for HA-50 in sterile phosphate buffered saline (PBS), pH 7.4 and stored in aliquots at −20° C. until use, without repeated freeze-thaw cycles. When stored at −20° C., the maximum shelf life of the frozen stock solutions was found to be approximately 2 months.

### Preparation of Gel Samples for Characterization.

Acellular gel samples were prepared to evaluate the mechanical properties and swelling of formulations with varied molecular weights and concentrations of HA. The detailed composition of formulations tested is given in Table 6. Hydrogel samples with an initial (pre-polymerization and pre-swelling) volume of 50 μL each were prepared in the manner reported in EXAMPLE 2 above. First, a 2× concentrated solution of PEG-binder adduct was prepared. PEG-VS was diluted from a 20% w/v stock solution to 5% w/v with 200 mM HEPES/2×PBS and ultrapure Water (Invitrogen) in a microtube and vortexed to mix. Then, stock solutions of peptide binders were added to the following concentrations: 3.0 mM PHSRN-K-RGD, 3.0 mM GFOGER, 1.0 BM-binder, and 1.0 FN-binder. The mixture was allowed to sit at room temperature for 30 min to allow the PEG-VS macromers to become functionalized with the pendant peptides via thiol-ene Michael addition reaction to form a PEG-binder adduct. After this, the HA-SH stock solution was diluted in PBS to double the final concentration in the gel, if needed (e.g., 0.5% w/v for the formulation with a final concentration of 0.25% w/v in the gel) and was vortexed 20-30 s to mix.

The PEG-binder adduct solution in HEPES/PBS was then added 1:1 by volume to the diluted HA-SH solution to form the pre-gel solution. The pre-gel solution was immediately vortexed 10-15 s to mix and 50 uL was added to round molds identical to those used for preparing the PEG-peptide gel samples. The hydrogel samples in molds were allowed to polymerize for 30 min in a humidified chamber at 37 C and 5% CO₂. The gels were then removed from the molds and placed in PBS to swell.

TABLE 7
Hybrid hydrogel formulations with varied molecular weights tested

| | [PEG- | | | | | | | | [HA- |
|---|---|---|---|---|---|---|---|---|---|
| | VS] | | | [PHSRN- | | [BM- | [FN- | | SH] |
| | (% | [HEPES] | | K-RGD] | [GFOGER] | binder] | binder] | | (% |
| Formulation | w/v) | (mM) | [PBS] | (mM) | (mM) | (mM) | (mM) | HA used | w/v) |
| A | 2.5 | 100 | 1X | 1.5 | 1.5 | 0.5 | 0.5 | HA-10 | 1.00 |
| B | 2.5 | 100 | 1X | 1.5 | 1.5 | 0.5 | 0.5 | HA-10 | 0.40 |
| C | 2.5 | 100 | 1X | 1.5 | 1.5 | 0.5 | 0.5 | HA-10 | 0.20 |
| D | 2.5 | 100 | 1X | 1.5 | 1.5 | 0.5 | 0.5 | HA-50 | 1.20 |
| E | 2.5 | 100 | 1X | 1.5 | 1.5 | 0.5 | 0.5 | HA-50 | 1.00 |
| F | 2.5 | 100 | 1X | 1.5 | 1.5 | 0.5 | 0.5 | HA-1500 | 1.00 |
| G | 2.5 | 100 | 1X | 1.5 | 1.5 | 0.5 | 0.5 | HA-1500 | 0.20 |

Volumetric Swelling Characterization. Post-polymerization, gel samples were allowed to swell to equilibrium (no net change in sample mass/volume) in divalent cation-free Phosphate Buffered Saline (PBS), pH 7.4 for 18-24 h at 37° C. and 5% CO₂, to approximate cell culture conditions. To assess volumetric swelling, the samples were removed from the PBS, excess moisture on the surface of the gel was removed, and the sample was moved to a glass microscopy slide surface-treated with SIGMACOTE® to prevent sticking and damage to the gel sample. The diameter and height in mm of the swollen gel sample was measured and used to calculate the volume in μL of the swollen gel at equilibrium (i.e., maximally swollen). To calculate the volumetric swelling factor (VSF), the volume (uL) of the equilibrium-swollen gel sample was divided by the initial volume of the sample (50 uL).

Characterization of Gel Mechanical Properties. The mechanical properties of the gel were assessed via shear oscillatory rheology using an Anton Paar MCR 302 rheometer with an 8 mm parallel plate measuring geometry and a Peltier plate with an air-controlled temperature regulation system. Equilibrium-swollen gel samples were placed on the Peltier plate sample stage, pre-warmed to 37° C., and the measuring geometry was lowered to 70-80% of the height of the equilibrium swollen sample. This compression of the sample prevented sample slippage during measurement. The sample was subject to 180 s time sweep at a frequency of 1 Hz and strain of 5% at 37° C. The storage and loss moduli were averaged over the time sweep for each sample. Those values for each sample were averaged to yield the average for the formulation (n=3), reported with the standard deviation. Frequency and amplitude sweeps were performed to verify that the testing parameters were within the linear viscoelastic regime (not reported).

### Results

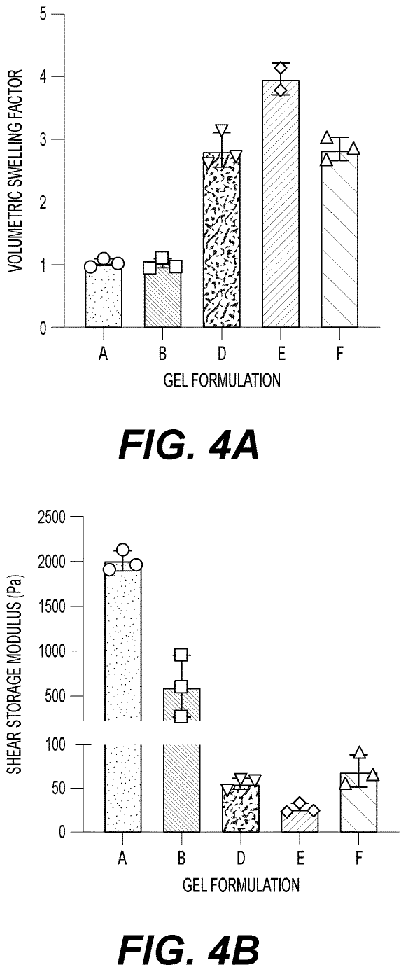

Of the formulations tested (A-H, listed in Table 7), two formulations, C and G, did not gel and were not subject to further characterization. The volumetric swelling factor, the ratio of the volume of the fully swollen gel at equilibrium to the starting volume of the gel immediately after polymerization, and the shear storage modulus of the remaining formulations were evaluated. The results are shown in FIG. 4A-B.

Generally, formulations made with HA-10 exhibited a lower volumetric swelling factor than formulations made with HA-50 or HA-1500 (FIG. 4A). This is especially notable when comparing the 3 formulations each made with 1.0% w/v HA-SH, but of varying molecular weights: A (HA-10), E (HA-50), and F (HA-1500). The gel formulations made with HA-10 also were the stiffest gels, according to their shear storage modulus (FIG. 4B). This is to be expected, as HA-10 is the most hydrophobic molecule out of the three, and therefore would interact the least with water, leading to less swelling. Surprisingly, the formulations that gelled that were made with HA-50 and HA-1500 exhibited similar properties in terms of swelling and shear storage modulus.

However, the physical properties of the HA are not the only contributing factor in the properties of the gel. The degree of crosslinking, which is related to the concentration of available thiols on the HA molecule, is also one determinant of gel properties. The concentration of thiol groups in the pre-gel solution can be estimated based on the concentration and degree of substitution of the HA, along with the molecular weight of the monomer of HA (one D-glucuronic acid group and one N-acetyl-D-glucosamine group, MW=379.32) which is constant. Therefore, while the gels made with 1.0% w/v HA all contain approximately 2.64 mM of HA monomers, there are differences in the concentration of thiols between the three groups, with 0.132 mM in the HA-1500 gel, 0.264 mM in the HA-50 gel, and 0.792 mM in the HA-10 gel Additionally, as a different comparison, formulations B and D are estimated to contain roughly the same amount of thiol groups, 0.316 mM, based on the concentration and degree of substitution of the HA used for each.

### Example 4: Culture of Isolated Primary Human Adipocytes

Long-term culture of adipocytes, particularly primary human adipocytes, has long been technically challenging. Notably, when conventional adherent cell culture methods are used, adipocytes float, do not adhere to the culture surface, and die. One alternative to using adipocytes has been to use adipocyte-like differentiated cells, including human pre-adipocytes and murine pre-adipocytes or cell lines such as 3T3-L1 or C3H10T1/2 cells, but these cells have limited resemblance to primary, mature adipocytes (Ruiz-Ojeda et al. Int. J. Mol. Sci., 2016) https://doi.org/10.3390/ijms17071040. Alternatively, other culture methods have been developed for primary adipocytes. One such method is adherent ceiling cultures, where cells are ‘floated’ to the top surface of a culture flask filled with liquid media and adhere on that surface, but those cells then de-differentiate and lose their adipocytic phenotype over the course of 2 weeks (Sugihara et al. Differentiation, https://doi.org/10.1111/j.1432-0436.1986.tb00381.x 1986). Recently, Harms et al. have reported another method using membrane mature adipocyte aggregate cultures, but this method is highly technically challenging and difficult to reproduce (Harms et al. Cell Reports, 2019 https://doi.org/10.1016/j.celrep.2019.03.026). 3D culture of primary adipocytes via encapsulation in a hydrogel matrix offers an accessible alternative method which would potentially maintain the adipocytic phenotype for time periods on the order of several weeks. The goal of this pilot study was to compare the fitness for primary adipocyte culture of PEG-HA hybrid hydrogels, previously published peptide-crosslinked PEG hydrogels (Hernandez-Gordillo et al., Biomaterials, 2020 https://doi.org/10.1016/j.biomaterials.2020.120125), and a commercially available hydrogel for 3D cell culture, HyStem-C.

### Materials and Methods

Materials. 20 kDa, 8-arm PEG-VS and ~300 kDa HA-SH with a molar degree of substitution ~20-30% were obtained commercially and reconstituted as detailed in Example 2. Concentrated HEPES/PBS stock solutions were prepared as described in Example 2. Custom-synthesized peptides detailed in Table 1 described were ordered and prepared in stock solutions in Example 2. PBS in solution, pH 7.4, was purchased from Gibco (Catalog #10010-023). Krebs buffer was prepared as a 5× stock solution containing 552 mM sodium chloride (Millipore Sigma S5886), 24 mM potassium chloride (Fisher Scientific, P217), 6 mM potassium phosphate (VWR 7100), 6 mM magnesium sulfate heptahydrate (VWR 0662), and 12.6 mM calcium chloride dihydrate (Millipore Sigma C7902) in MilliQ distilled water. Krebs buffer stock solution was diluted 5-fold in MilliQ distilled water before use at 1×. HYSTEM-C®, a commercially available HA-collagen gel for 3D cell culture, was purchased from Sigma-Aldrich (HYSC020) and used according to manufacturer's instructions with slight modification, as described in the following section. HYSTEM-C® is packaged as a kit containing three reagents: GLYCOSIL®, a thiolated HA also used in the preparation of the PEG-HA hydrogels, GELIN-S®, a thiolated porcine gelatin, and EXTRALINK®, a PEG-diacrylate. Cell culture media used for the adipocytes consisted of basal medium Dulbecco's modified Eagle's medium/Ham's nutrient mixture F12 (DMEM/F12; Gibco 10565-018), 10% heat-inactivated fetal bovine serum (R&D Systems), and 1× penicillin/streptomycin (Gibco 15140-148)/.

Gel encapsulation of adipocytes. Adipocytes were isolated from tissue samples according to an adaptation of the method published by Alexandersson et al. (J. Vis. Exp., 2020 https://dx.doi.org/10.3791/60485) then were washed with Krebs buffer and centrifuged at 50 RCF for 3 min. The centrifuged mixture yielded a non-adipocyte cell pellet at the bottom, an aqueous phase, an oily phase at the top, and a layer of adipocytes between the aqueous and oily phases. As much of the oily phase of free lipids on top of the cells was then aspirated manually with a needle and syringe and disposed. Next, the aqueous phase and the non-adipocyte cell pellet at the bottom of the tube were removed via needle and syringe. The remaining adipocytes are roughly separated by size, with the largest cells at the top and the smallest cells at the bottom. Using a wide-bore pipette tip, cells from the middle were collected and moved to a sterile microtube.

To encapsulate the cells in the HyStem-C gel, the contents of the kit (Glycosil, Gelin-S, and Extralink) were reconstituted according to the manufacturer's instructions. Glycosil and Gelin-S were combined in a 1:1 ratio by volume. Adipocytes were added so that they comprised 15% by volume of the final pre-gel mixture and pipetted up and down several 5 times to mix. 30 μL of Extralink solution per 125 μL of Glycosil/Gelin-S mixture used was added to the center of a well of a 24 well plate. The Glycosil/Gelin-S/adipocyte mixture was then added directly on top of the Extralink solution in the well and was mixed by gently shaking the well plate from side to side. The well plate was incubated at 37 C and 5% CO₂ in a humidified incubator for 1 h to allow samples to gel. Culture media was then added to the samples and they were moved back to the incubator.

The PEG-peptide gel formulation used to culture the adipocytes was 3% w/v (1.50 mM) PEG-VS, 100 mM HEPES/1×PBS, 1.5 mM PHSRN-K-RGD, 1.5 mM GFOGER, 0.5 mM BM-binder, 0.5 mM FN-binder, and 2.70 mM LW. To encapsulate the cells in the gel, PEG-VS, HEPES/PBS, PHSRN-K-RGD, GFOGER, BM-binder, and FN-binder stock solutions were added to a microtube and vortexed to mix. The mixture was allowed to react at room temperature for at least 15 min to form the PEG-binder adduct. A volume of ultrapure water was added to the PEG-binder adduct solution sufficient to bring the final pre-gel solution (including adipocytes and LW) to the desired final volume, before adding adipocytes. Adipocytes were added so that they comprised 15% by volume of the final pre-gel mixture. The PEG-binder adduct, ultrapure water, and adipocyte mixture was gently pipetted up and down to mix. Then LW stock solution was added to obtain a final concentration of 2.70 mM in the pre-gel solution (0.45 crosslinking ratio). As used herein, “crosslinking ratio” here is the ratio of the concentration of reactive groups on the bifunctional crosslinking molecule in the mixture to the concentration of total ‘arms’ of the multiarm PEG in the mixture (i.e., mM concentration of the PEG macromer×8 for the 8-arm PEG-VS).

The pre-gel mixture was pipetted up and down to mix, and then 12 uL droplets of pre-gel mixture were deposited per well in a 24-well plate. The well plate was moved to a humidified incubator at 37 C and 5% CO₂ for 30 min to polymerize. Culture media was added to each gel sample and then the well plate was returned to the incubator.

The PEG-HA gel formulation used to culture the adipocytes was 1.65% w/v (0.825 mM) PEG-VS, 66.7 mM HEPES/0.67×PBS, 0.165% HA-SH (GLYCOSIL®), 1 mM PHSRN-K-RGD, 1 mM GFOGER, 0.33 mM BM-binder, and 0.33 mM FN-binder. To encapsulate the adipocytes in the PEG-HA gel, first a solution of 5% w/v PEG-VS, 200 mM HEPES/2×PBS, 3 mM PHSRN-K-RGD, 3 mM GFOGER, 1 mM BM-binder, and 1 mM FN-binder was prepared in a microtube. This solution was mixed thoroughly via vortex and allowed to react at room temperature for at least 15 min to form the concentrated peptide-functionalized PEG macromer. Meanwhile, the HA-SH was diluted in PBS in a microtube and mixed thoroughly via vortex. Adipocytes were added to the diluted HA-SH so that they comprised 15% by volume of the final pre-gel mixture and were mixed gently by pipetting up and down. The concentrated peptide-functionalized PEG macromer was then added to the HA-SH/adipocyte mixture so that it comprised 33% of the final pre-gel mixture by volume. This was mixed by pipetting up and down several times, and immediately after 12 μL droplets of pre-gel mixture were deposited per well in a 24-well plate. The well plate was moved to a humidified incubator at 37° C. and 5% C02 for 30 min to polymerize. Culture media was added to each gel sample and then the well plate was returned to the incubator.

For all gel formulations, media was changed every 2 days.

Live/Dead staining and imaging. Gel samples were removed from the incubator and the culture media was removed. Samples were washed once with excess PBS, and then stained with LIVE/DEAD viability/cytotoxicity stain (Invitrogen, Carlsbad, CA, USA) diluted 1:1000 in PBS for 20 min in the incubator at 37° C. Epifluoresence images were collected at 4× magnification.

### Results

Initial attempts at 3D culture of primary adipocytes in PEG-HA hydrogels used a formulation consisting of 2.5% w/v PEG-VS+0.25% w/v HA-SH+100 mM HEPES/1×PBS+1.5 mM PHSRN-K-RGD+1.5 mM GFOGER+0.5 mM BM-binder+0.5 mM FN-binder. However, when the adipocytes were added to the pre-gel solution, the mixture gelled instantaneously and could not be pipetted, which made it impossible to deposit gel-encapsulated cells into the well plate. This rapid gelation was likely due to phase separation induced by free lipids in the liquid containing the cells. A modified, 33% less concentrated formulation was subsequently used, which did not gel immediately upon addition of adipocytes and had a working time (i.e., time before the pre-gel solution became too viscous to pipette) on the order of several minutes, allowing for thorough mixing and the encapsulation of the adipocytes for 3D culture.

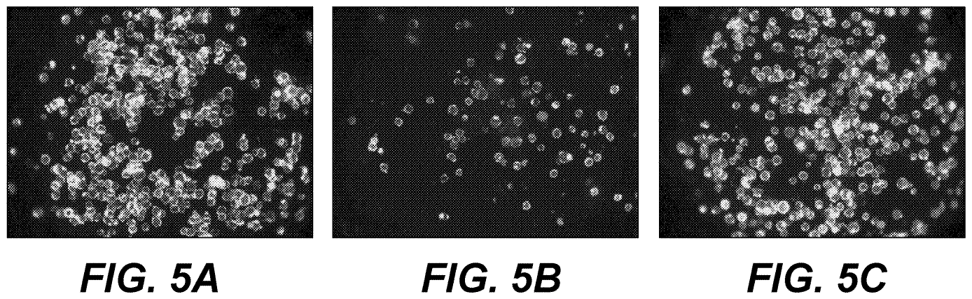

Live/Dead staining of primary adipocytes after 7 days in culture indicates a higher viability in the control condition, HYSTEM-C® gel (FIG. 5A), and the PEG-HA condition (FIG. 5C) compared to the PEG-peptide condition (FIG. 5B). This suggests that the PEG-HA hydrogel formulation tested better supports primary human adipocyte viability in 3D culture than the peptide-crosslinked PEG hydrogel formulation. Because cell viability is also high in the HYSTEM-C® hydrogel, the adipocytes may require HA in their microenvironment to survive 7 days in 3D culture.

### Example 5: Culture of Human iPSC-Derived Neurons

Endometrial ectopic lesions characteristic of endometriosis and adenomyosis exhibit a high degree of innervation with peripheral neurons. The impact of these nerve cells on the growth of the lesion and the pain experienced by patients diagnosed with endometriosis and adenomyosis has yet to be elucidated. In vitro models of endometriotic lesions which include nociceptors, peripheral nerve cells specialized to respond to noxious stimuli, would therefore be a valuable tool to interrogate molecular and cellular mechanisms involved in interactions between endometrial cells and nerve cells. However, developing highly reproducible culture conditions which support the growth and maintenance of both nerve cells and endometrial cells is non-trivial.

This work details a pilot study conducted using the nerve-on-a-chip model developed by Moore et al. for the culture of induced pluripotent stem cell (iPSC)-derived nociceptors (Khoshakhlagh et al., J. Neural Eng., 2018, DOI:10.1088/1741-2552/aae129) (Pollard et al., Sci. Adv. 2021, DOI: 10.1126/sciadv.abj2899) to test the support of iPSC-nociceptor growth by synthetic PEG-peptide and hybrid PEG-HA gels compared to the current standard, MATRIGEL®.

### Materials and Methods

Materials. 20 kDa, 8-arm PEG-VS and ~300 kDa HA-SH with a molar degree of substitution ~20-30% were obtained commercially and reconstituted as detailed in Example 2. Concentrated HEPES/PBS stock solutions were prepared as described in Example 2. Custom-synthesized peptides detailed in Table 1 described were ordered and prepared in stock solutions in Example 2.

Nerve-on-a-chip model. Details of the nerve-on-a-chip model, including the method of fabrication of the growth-impermissive (poly(ethylene glycol)-di-methacrylate, PEGDMA) hydrogel mold used to hold the growth-permissive hydrogel, as well as its dimensions and composition are included in Khoshakhlagh et al., Journal of Neural Engineering, 2018. DOI: 10.1088/1741-2552/aae129. Methods for nerve spheroid formation and implantation of cells in the nerve-on-a-chip constructs as well as details of MATRIGEL preparation are reported by Pollard et al., Science Advances, 2021. DOI: 10.1126/sciadv.abj2899. iPSC-derived nociceptors (i.e., peripheral neurons sensitive to noxious signals) were sourced from Anatomic, Inc. and maintained in culture according to the manufacturer's protocols.

Cell encapsulation in PEG-peptide and PEG-HA hydrogels. In brief, an acellular pre-gel mixture of a peptide-crosslinked PEG hydrogel (“PEG-peptide”) was prepared of 2.6% w/v PEG-VS+100 mM HEPES/1×PBS+2.81 mM LW (0.54 branching ratio)+1.5 mM PHSRN-K-RGD+1.5 mM GFOGER+0.5 mM BM-binder+0.5 mM FN-binder according to the method reported in EXAMPLE 2 for preparing gel samples for characterization. Additionally, an acellular pre-gel mixture of an HA-crosslinked hybrid hydrogel (“PEG-HA”) of 2.5% w/v PEG-VS+0.25% w/v HA-SH+100 mM HEPES/1×PBS+1.5 mM PHSRN-K-RGD+1.5 mM GFOGER+0.5 mM BM-binder+0.5 mM FN-binder was prepared using the method reported in EXAMPLE 2 for preparing gel samples for characterization. However, at the final step, the pre-gel solution was added to the central channel of the PEGDMA hydrogel mold, and then one spheroid per construct was immediately added via pipette to one end of the barbell-shaped channel. With the spheroids added, the constructs were moved to the humidified incubator at 37° C. and 5% C02 for 30 min to allow the gel to polymerize. After that, media was added around the constructs by pipetting into the gap between the Transwell plate insert containing the construct and the wall of the well beneath it. Samples were cultured for 37 days before fixing with 4% paraformaldehyde, immunostaining for beta 3 tubulin, and staining for actin using ActinRed.

### Results

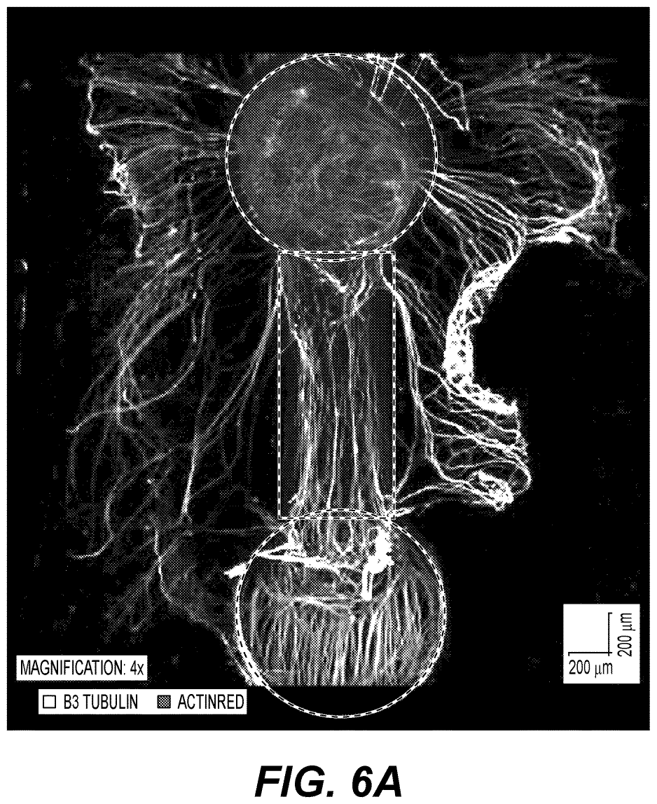

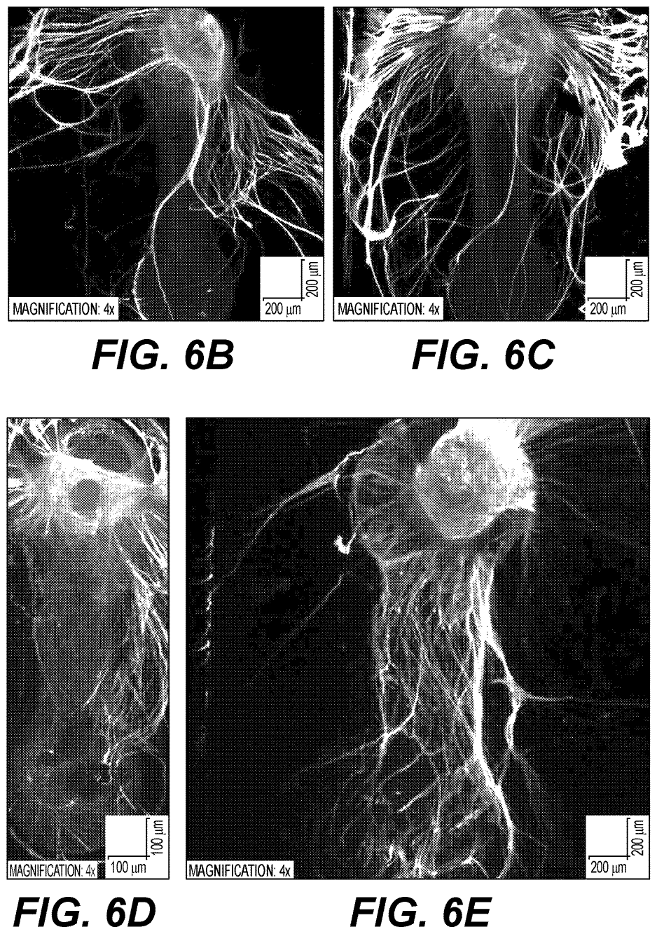

Constructs fixed and stained for beta 3 tubulin (green) and f-actin (red) after 37 days in culture demonstrate cell growth beyond the initially implanted spheroids in all 3 hydrogels. The MATRIGEL® control, shown in FIG. 6A, demonstrated the extension of processes from the nerve cells, identified by beta 3 tubulin-positive staining (green) as opposed to f-actin-positive staining (red), from the spheroid down the length of the central channel of the barbell-shaped growth-permissive gel compartment (boundaries indicated by the white dashed line). Additionally, the MATRIGEL®. condition exhibits some of these processes extending outside the bounds of the central, MATRIGEL®.-filled channel of the construct. These processes appear in confocal microscopy to be growing over the top of the outer PEGDMA hydrogel. MATRIGEL®. is the current standard growth-permissive gel used in the nerve-on-a-chip model (Pollard et al., Science Advances, 2021. DOI: 10.1126/sciadv.abj2899), and these results in the MATRIGEL® control are in line with previous experiments. In contrast, as shown in representative images in FIG. 6B-6C, spheroids seeded in PEG-peptide gel in the central compartment exhibited beta 3 tubulin-positive processes extending outside the boundaries of the central channel, on top of the surrounding PEGDMA hydrogel, but not within the PEG-peptide gel in the central channel. Furthermore, f-actin-positive (red), beta 3 tubulin-negative (i.e., non-neuronal) cells appeared to cover the bottom of the channel. These cells are assumed to have migrated from the implanted spheroids, but the details of their phenotype are unknown at time of writing. Lastly, spheroids seeded in PEG-HA gels in the central channel exhibited widely divergent morphology among the replicates in the experiment (n=3), as demonstrated in two representative images of fixed and stained constructs in FIG. 6D-6E. Two of the three replicates resembled the image on the left, with an abundant layer of f-actin-positive (red), beta 3 tubulin-negative cells along the bottom of the central channel, with beta 3 tubulin-positive (green) cell processes appearing to extend beyond the bounds of the barbell-shaped growth-permissive gel channel and along the surface of the PEGDMA outer gel, similar to the PEG-peptide gel samples. However, one of the three PEG-HA gel constructs, shown to the right in FIG. 6D-6E, exhibited a high degree of extension of beta 3 tubulin-positive (green) cellular processes along the length of the central gel channel, with relatively few extending on the surface of the PEGDMA gel, outside the boundaries of the central gel channel. Additionally, this sample exhibited few, if any, actin-positive, beta 3 tubulin-negative cells growing along the bottom of the central channel. The reason for the stark differences from sample-to-sample in the PEG-HA gel condition is uncertain, but may be due to incomplete mixing of PEG-rich and HA-rich phases during the preparation of the pre-gel solution, which can be addressed with a longer and more vigorous mixing step in the protocol. The storage conditions of the HA-SH stock solutions may also contribute to incomplete mixing between PEG-rich and HA-rich phases in gel preparation, as HA-SH stock solutions stored at −20 C exhibit increasing viscosity when stored for over 2 months, which was the case here. Further testing has demonstrated that the shelf life of the HA-SH stock solutions, without this viscosity increase, is significantly increased by storing the solutions at −80 C. Ongoing investigations with an updated pre-gel solution preparation protocol for the PEG-HA gel. Additionally, ongoing experiments are testing the impact of adding 0.5 mM each of two synthetic peptides with cell-adhesive sequences derived from the alpha and beta chains of laminin-111 into both the PEG-peptide and the PEG-HA hydrogel formulations.

### Example 6: Development of a Synthetic Hydrogel to Support Vascularized Microphysiological Systems

### Materials and Methods

### Preparation of Hydrogel Components

8-arm 20 kDa poly(ethylene glycol) (PEG) macromers functionalized with vinyl sulfone (PEG-VS) were purchased from JenKem Technology (Beijing). All peptides were custom synthesized and purified (>95%) by CSBio (Milpitas, CA), CPC Scientific (Sunnyvale, CA), or GenScript (Piscataway, NJ). Peptides used in this study include: a dithiol crosslinking peptide with an MMP-cleavable substrate (Ac)-GCRDLPRTGGPQGIWGQDRCG(Am) (LW); a fibronectin-derived peptide containing the canonical RGD motif as well as the PHSRN synergy site, as previously described²⁰, (Ac)-PHSRN-GGG-K(GGGERCG-Ac) GGRGDSPY(NH₂) (PHSRN-K-RGD); a collagen-derived peptide, (Ac)-GGYGGGPGGPPGPPGPPGPPGPPGFOGERGPPGPPGPPGPPGPPGPC-(Am) (GFOGER); a peptide with affinity for sequestering basement membrane proteins type IV collagen and laminin, Ac-GCREISAFLGIPFAEPPMGPRRFLPPEPKKP-NH2 (BM Binder); a peptide with affinity for sequestering cell secreted fibronectin, Ac-KKGCREPLQPVYEYMVGV-Amide (FN Binder); and a peptide derived from binding motif of laminin, Ac-GCREYIGSR-Amide (YIGSR).

PEG-VS was dissolved in Ultrapure water (Invitrogen) using a volumetric flask to create a 10% w/v solution and aliquoted and stored at −80° C. until use. All peptides were reconstituted in sterile UltraPure water (Invitrogen) in a biosafety cabinet at a target concentration of 30 mM for PHSRN-K-RGD, GFOGER, YIGSR and LW; 15 mM for FN binder; 20 mM for BM binder. To fully dissolve YIGSR a small amount of DMSO (<10% of total volume) was added. To fully dissolve FN binder the pH must be adjusted to a final pH of 5-7 using a concentrated NaOH solution. Peptide solutions were sterile-filtered, aliquoted, and stored at −80° C. until use in the gel. All aliquots were used for less than 3 freeze-thaws. Peptide concentrations were measured using Ellman's assay (Sigma 22582) for GFOGER, 205 nm absorbance for PHSRN-K-RGD, LW, BM binder, and FN binder, and 280 nm absorbance for YIGSR.

### Cell Culture

GFP-labeled Human umbilical vein endothelial cells (HUVECs) and GFP-labeled human uterine microvascular endothelial cells (HUTMVECs) were purchased from Angio-Proteomie (cAP-0001GFP and cAP-0023GFP). Normal Human Lung Fibroblasts (NHLFs) were purchased from Lonza (CC-2512). Endometrial stromal cells (ESCs) and endometrial epithelial organoids (EEOs) were from an endometrial tissue bank and isolated and expanded using previously published methods²⁰ under IRB-P0012994. HUVECs were cultured in Vasculife VEGF Endothelial Medium (Lifeline Technologies LL-0003), HUTMVECs were cultured in Vasculife VEGF-Mv Endothelial Medium (Lifeline Technologies LL-005), and fibroblasts were cultured in Fibrolife S2 Fibroblast Medium (Lifeline Technologies LL-0011). ESCs were cultured in DMEM/F12 with 5% charcoal-stripped fetal bovine serum (FBS, Biotechne #511650), 1% Penstrep (Gibco 15140122), and 1 nM Estradiol (Sigma-Aldrich E8875). EEOs were cultured in Matrigel Growth Factor Reduced, Phenol Red-free (Corning 356231) prior to using in the synthetic hydrogel in a media previously described²⁰. All cell types were used between passages 5-9.

### Monocyte Isolation

Human monocytes were isolated from healthy donors, either isolated from buffy coats obtained from Massachusetts General Hospital or from peripheral blood Leukopaks (StemCell Technologies 200-0092). CD14⁺ monocytes were isolated from buffy coats as previously described⁷⁶⁻⁷⁷. First, peripheral blood mononuclear cells (PBMCs) were isolated via density gradient centrifugation with Lymphoprep (STEMCELL 18061). PBMCs were washed with MACs buffer: PBS supplemented with 2% charcoal-stripped FBS (Gemini Bio 100-119) and 2 mM EDTA (Corning 46-034-CI), and isolated using a EasySep Human CD14 Positive Selection Kit II (STEMCELL 19359) according to the supplier's instructions. Leukopaks were processed similarly without the initial density gradient step. Once isolated, CD14+ cells were cryopreserved at 10 million cells/vial in freezing media (90% charcoal-stripped FBS with 10% DMSO).

### Macrophage Differentiation

Cryopreserved CD14+ monocytes were thawed into RPMI 1640 media (Gibco 32404014) supplemented with 10% charcoal-stripped FBS, 1% Pen-Strep (ThermoFisher 15140148), GlutaMAX (ThermoFisher 35050061), then seeded in dishes at a density of 1 million cells per mL in 10 mL of complete media with 50 ng/mL human M-CSF (BioLegend 574804). Cells were incubated at 37° C./5% CO₂ for a total of 6 days, with a media change on day 3. To harvest cells for downstream experiments, cells were washed with PBS and incubated in MACs buffer at 4° C. for 20 min followed by gentle washing with a p1000 before collection and centrifugation at 300 G for 5 min. Macrophages were stained by incubating with CellTrace™ Far Red Cell Proliferation Kit (Invitrogen C34572, 1:1000) in a 50:50 solution of MACs buffer and PBS for 20 min at 37° C. and washed with 25× volume of MACs buffer before cell counting for downstream experiments.

### Hydrogel Creation

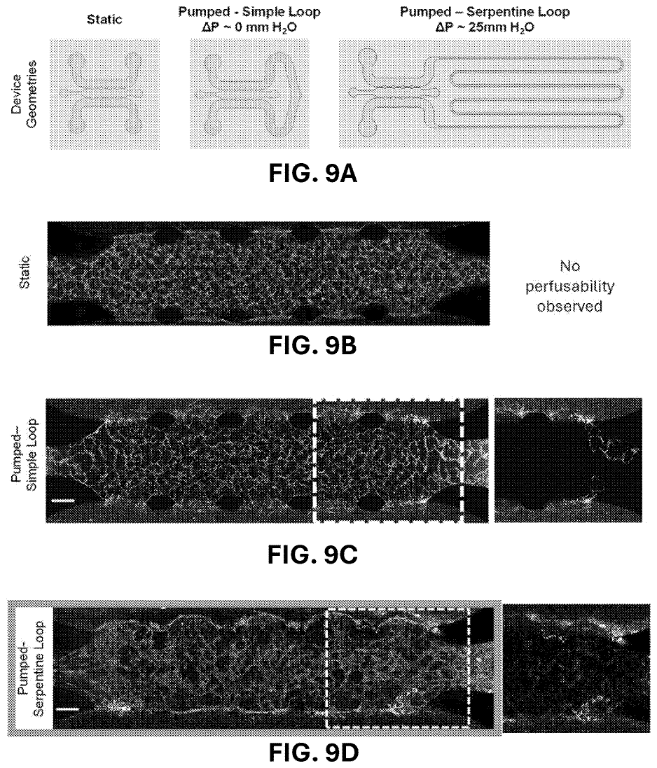

Synthetic hydrogels were assembled using PEG weight % and crosslinking ratios denoted throughout the text. In all studies, PHSRN-K-RGD and GFOGER were used at 1.5 mM and BM binder and FN binder were used at 0.5 mM. YIGSR was added to the vascular formulation at a concentration of 0.6 mM. PEG-VS concentrations were 11.7 mM for the 3.0 wt % condition and 14.04 mM for the 3.6 wt % condition. To create the hydrogel, first PEG-VS macromers were functionalized with the monothiol pendant peptides (PHSRN-K, RGD, GFOGER, BM binder, FN binder, YIGSR) in a buffered solution, with 10% of the volume 10×PBS+1 mM HEPES, pH 8.2. Briefly all components were added at appropriate concentrations, vortexed thoroughly, and allowed to react via a Michael-type addition for 30 minutes at room temperature. If including cells (all studies except rheology in FIG. 9), the desired cells were lifted and pelleted using standard protocols during the 30-minute functionalization step. Cells were mixed at the desired concentration for a certain volume of gel (ex. 18M HUVECs/mL, 7; 1 ratio—for a 40 μL gel this is 720,000 HUVECs and 102,857 NHLFs in a pellet) and pelleted using centrifugation (300 g×5 min) in a microcentrifuge tube. Carefully, all media was removed from the cell pellet after centrifugation, and the pre-functionalized PEG solution was added to the cell pellet at an appropriate volume and mixed via gentle pipetting at least 15 times. Next, quickly the LW crosslinker was added to create a desired thiol-ene crosslinking ratio of 0.43 to 0.52 to the PEG-cell mixture. Again, the gel is thoroughly mixed using pipetting at least 15 times prior to loading the pre-gel solution into the device using a 10 μL pipet. Gel batches were typically prepared in 40 μL volumes to load 3-4 devices per batch. After loading, devices are placed at 37° C. in a humidified incubator for 30 minutes to allow gelation to occur via a Michael-type addition. Media was added to each device after the 30-minute gelation incubation. For experiments including EEOs, the EEOs were grown in Matrigel to an average diameter of approximately 100-150 μm before harvesting in Cell Recovery Solution (Corning 354253) to dissolve the Matrigel. Intact organoids were added at a concentration of 3 EEOs/μL of gel to the endothelial-stromal cell pellet before mixing with the functionalized PEG solution.

### Rheology

To measure the bulk mechanical properties of the hydrogel, 50 ul of the pre-gel mixture was pipetted into 8 mm silicone molds (0.5 mm thick) which were stuck to slides that had been coated with SigmaCote (Millipore Sigma SL2) and allowed to gel at 37° C. After gelation, hydrogel discs were removed from the silicone molds/glass slides using a plastic spatula and weighed using a balance to get the pre-swelling mass. The hydrogel disc was then placed in a 12-well plate with 2 mL of PBS. The plate was incubated for 24 hours in a 37° C. incubator to allow equilibrium swelling to occur prior to rheological testing. After swelling, the hydrogel discs were weighed on a balance to get the post-swelling mass to calculate the swelling factor. An Anton Paar rheometer (model MCR302) was used to measure rheological properties. The hydrogel discs were sandwiched between an 8 mm sandblasted parallel plate and sandblasted stage set to 37° C. The shear storage modulus (G′) was measured by performing a time sweep for 180 s at 1% strain and 1 rad/s angular frequency. The elastic modulus was calculated from the measured storage modulus in the linear elastic regime where E=2G′(1+v), with the assumption that v=0.5 for elastic materials.

### Device Fabrication

Polydimethylsiloxane (PDMS, Sylgard 184 Silicone Elastomer Kit, Dow) devices were fabricated using a 10:1 ratio of silicone elastomer base to curing agent. Briefly the PDMS mixture was poured in a silanized stiff PDMS negative mold (5:1 ratio of silicone elastomer base to curing agent) and allowed to cure for at least 2 hours at 65° C. The PDMS negative molds were created from a silanized positive milled acrylic mold (CAD file available upon request). All devices had a central 1.8 mm wide channel flanked by two media channels (FIG. 19). The gel-/media-channel interface featured a 0.1 mm phase guide and 8 posts (4 per side), which assisted with proper hydrogel loading. Serpentine devices also featured a narrow, serpentine channel (0.3 mm channel width) connecting both media channels. This narrow channel design created a pressure drop across the gel channel of approximately 32 mm H₂O at a flow rate of 1 μL/s, calculated with COMSOL as described below.

1.2 mm holes were punched at both ends of the gel channel in all devices. In static devices, four 6 mm holes were punched at the ends of both media channels. Once bonded these large punches acted as media reservoirs, allowing the device to hold 300 μL of media per device. In serpentine devices, two 1.2 mm holes were punched into the non-serpentine ends of the media channels. Once bonded sterile syringe tips were then inserted into these holes connecting the device to an external peristaltic pump and media reservoir.

Before bonding. all PDMS devices were washed sequentially with methanol, isopropanol, distilled water, and methanol, dried with an air hose, then autoclaved. Devices were bonded with the microfluidic channel side facing downwards, leaving only punched areas exposed. Static devices were bonded to sterile 6-well glass bottom plates (Cellvis P06-1.5H-N), while serpentine devices were bonded to sterile standard glass microscope slides. Every device was stored in an oven at 65° C. for at least one day prior to use.

### Pumped Device Setup

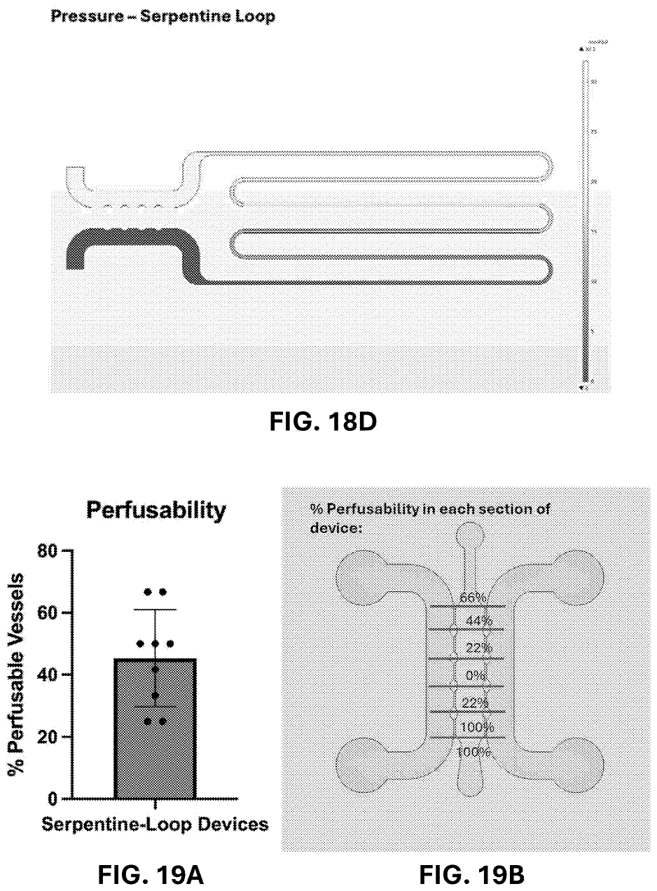

All pump circuit components were sterilized according to the methods outlined in FIG. 18C. These components were then assembled inside a biosafety cabinet (FIG. 18D), with the media reservoir intentionally left disconnected. Pump tubing was then fed into an Instech P625 peristaltic pump (Instech) and held within the pump by a 3D printed pump cap. 0.5 mL of media was then circulated through the pump circuit, allowing excess media to drain into a waste collection tube. After priming, the inlet and outlet connectors were attached to the media reservoir by the top port and side port respectively. An Eppendorf® protein LoBind tube (Millipore Sigma) with 1 mL of media was also secured into the media reservoir.

To connect the serpentine devices, one tube was detached from each pump circuit's syringe tip and affixed to a new, sterile syringe tip. These two syringe tips were then inserted into the pre-punched holes at the top of each device media channel. For the lifetime of each experiment, devices were stored inside PREempt-sterilized 3D printed imaging bases (as shown in the bottom image). Caps from three 15 mL conical tubes were secured into the base and filled with PBS to keep the base chamber humidified, then a sterile universal 96-well plate lid was placed on top of the base to seal the chamber. The full system was then transferred to a standard cell culture incubator, and the pumps were subsequently powered to deliver a constant volumetric flow rate of 1 μL/s.

The imaging base was designed to fit inside a Keyence BZ-X710 imaging microscope (Keyence). Pump caps were required to keep the pump tubing from falling out of the pump during extended use of the peristaltic pumps. Both were made from polylactic acid. CAD files for both parts are available upon request.

### COMSOL Modeling of Pressure and Fluid Shear Stress on Hydrogel

The pressure difference and shear stress on the hydrogel were numerically simulated using COMSOL Multiphysics version 6.3. Briefly, a steady, laminar, single phase flow assumption was used based on the Reynolds number (~1.5) in the media channels. The tissue chamber was not modeled as the flow through the hydrogel was assumed to be negligible, compared to the flow through the side channels. The shear stress at the interface between the hydrogel and media was calculated to be ~0.07 dyne/cm². This was calculated by multiplying the shear rate reported by the simulation with the dynamic viscosity of the fluid. The pressure difference between the two side channels was estimated to be ~32 mmH₂O, created by the hydraulic resistance to flow in the long serpentine connecting segment between the side channels. The fluid properties used in the simulation were taken from Poon⁷⁸, for media containing 5% FBS (density 1002 kg/m³, dynamic viscosity 0.862 mPa·s).

### Pressure Drop Experimental Measurements

Vertical column pressure drop measurements (FIG. 18A) were taken to verify that the serpentine narrow channel created a pressure gradient across the hydrogel. For testing, pump circuits were set up identically to a normal experiment and connected to serpentine devices loaded with a cell-free PEG gel. Tubing in the pump circuit was cut immediately before the device inlet and after the device outlet to insert 1/16″ tee union hose barb fittings (T-connector, Cole Parmer). Excess 0.031″ I.D. Tygon tubing was attached to the third barb of the T-connector and was loosely taped to the wall so that the tubing created parallel vertical columns. PBS was circulated through the device for 10 minutes before measuring the media height difference between the inlet and outlet vertical columns.

### Device Maintenance

Devices were imaged daily at 4× using a Keyence BZ-X710 microscope in the green channel. Media was changed daily, with 1 mL of media exchanged on the pumped devices and 300 μl of media for static devices (150 μl per side). Media was collected daily in Nunc™ 96-well U-bottom plates (Thermo Scientific 268200) and centrifuged at 1000 g×5 min and then transferred to a new plate for storage at −80° C. until downstream ELISA analysis.

### Media Composition for Experiments

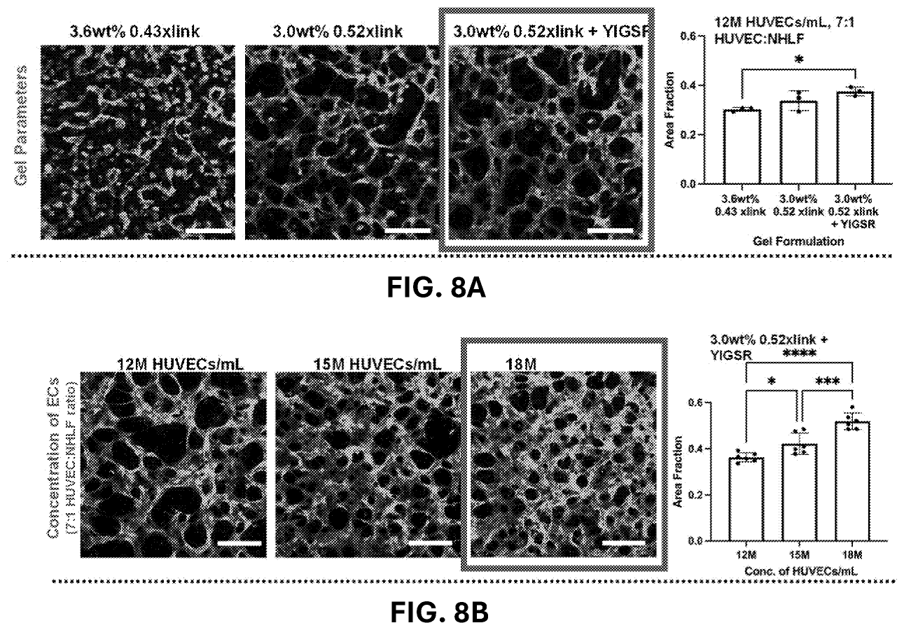

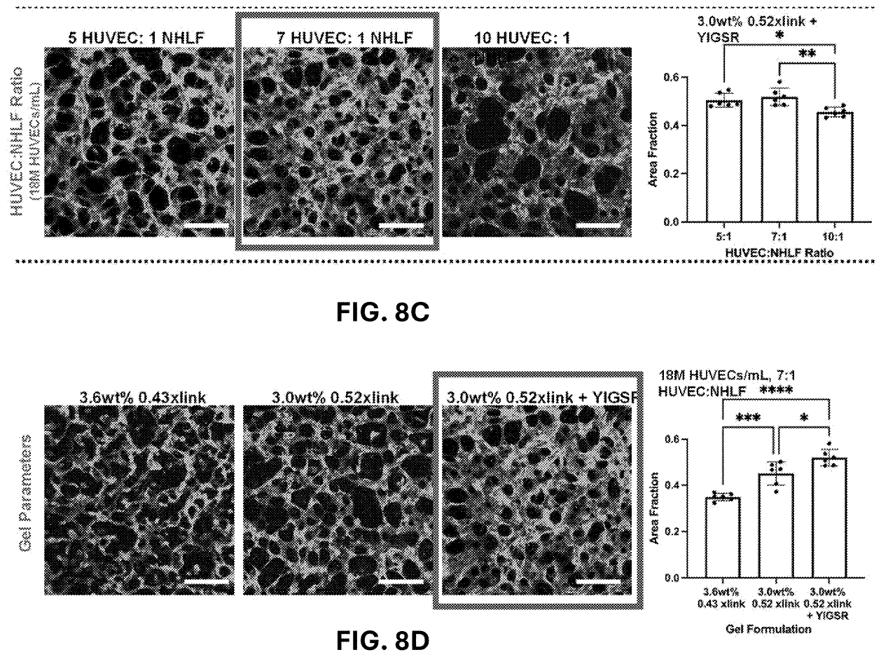

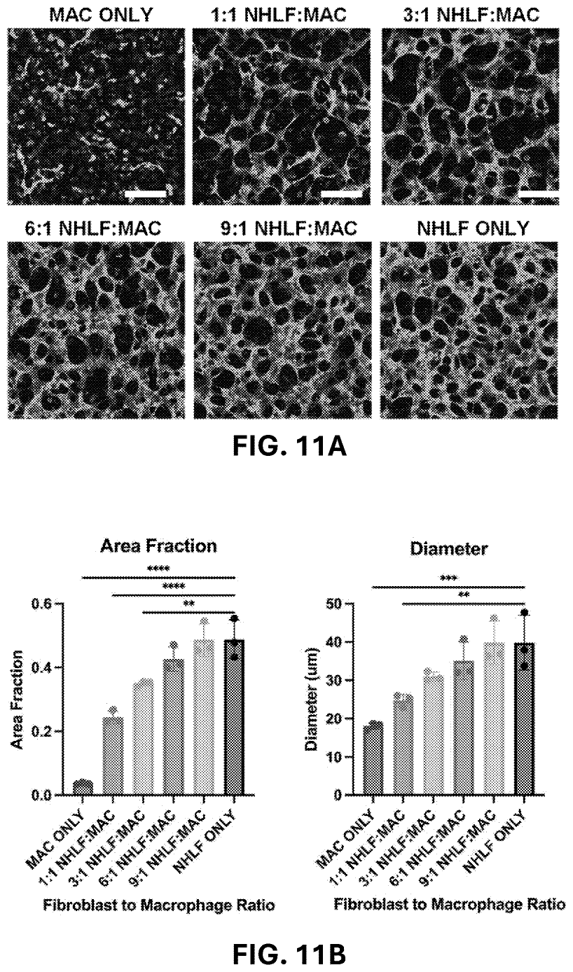

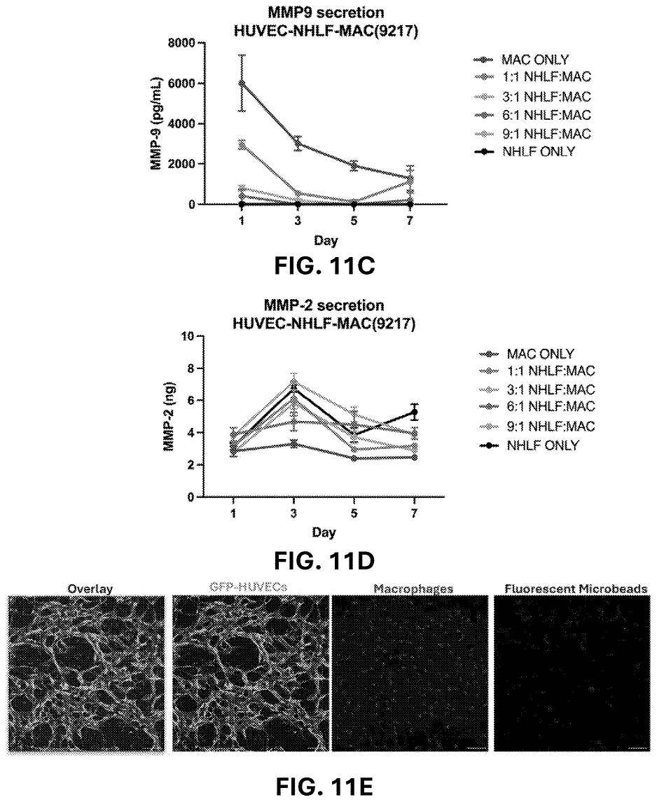

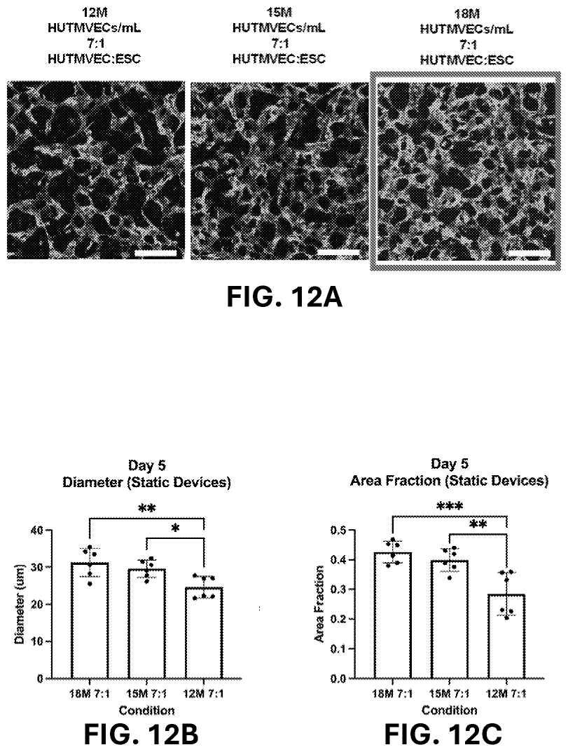

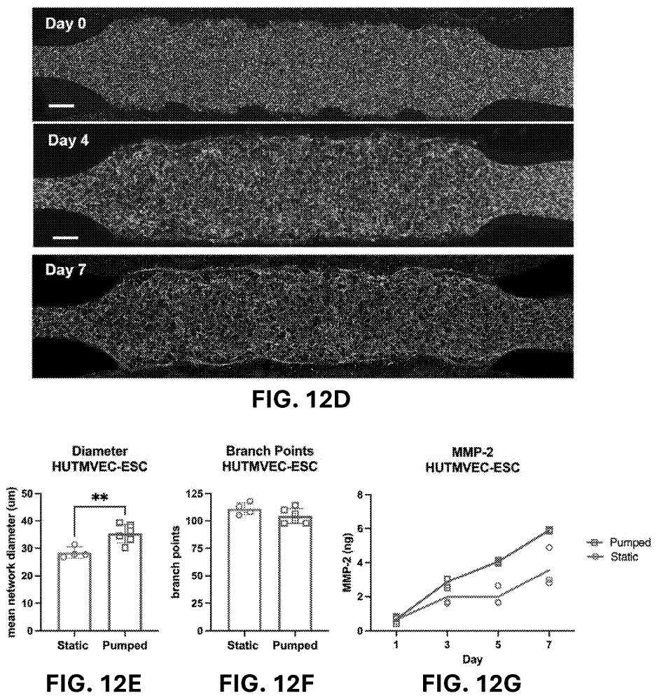

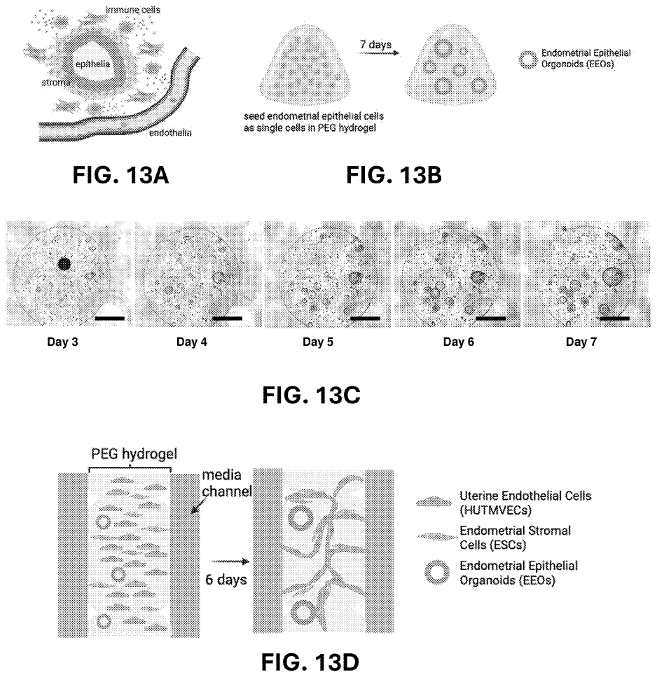

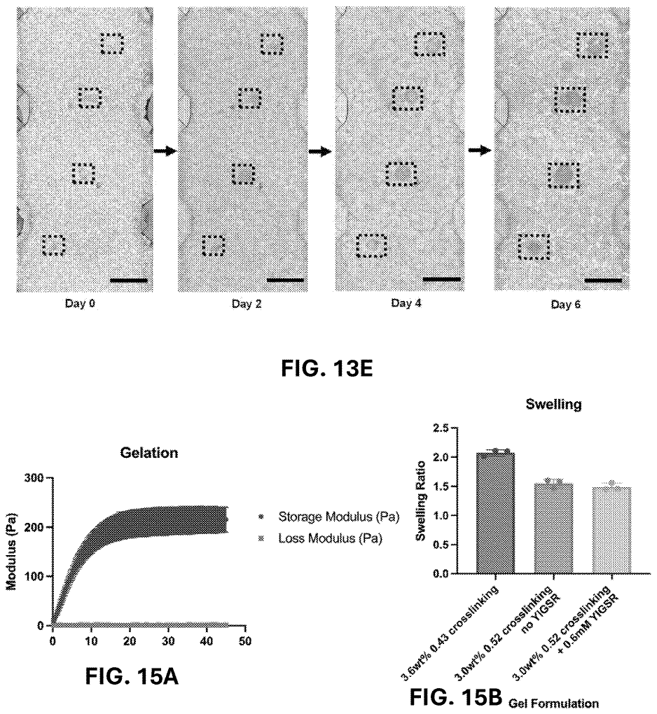

In HUVEC-NHLF experiments (FIGS. 8-10), devices were cultured with Vasculife VEGF Endothelial Medium (Lifeline Technologies LL-0003). In experiments containing macrophages (FIG. 11), the Hydrocortisone Hemisuccinate concentration was reduced to 500 nM to reduce the effect of cortisol on macrophage activity. In HUTMVEC-ESC experiments without organoids (FIG. 12), devices were cultured in Vasculife VEGF-Mv Endothelial Medium (Lifeline Technologies LL-005). In HUTMVEC-ESC experiments with organoids (FIG. 13) a 50-50 mixture of VL VEGF-Mv Endothelial Medium (kit FBS was replaced with charcoal-stripped FBS) and Endometrial Epithelial Organoid medium²⁰ without A8301 and Y-27632 with 10 nM E2 was used for the duration of the experiment. In all experiments, media was changed and collected daily.

### ELISAs

Human MMP-2 (R&D Systems DY902) and Human MMP-9 DuoSet (R&D Systems DY911) ELISAs were performed on spent medium. Manufacturer protocols were adapted to be performed in a 384-well plate to minimize the sample volume needed. Standard curves were fit to a 4-parameter logistic curve using MATLAB or GraphPad Prism. Technical duplicates were performed for all samples and standard curves were performed in at least triplicate. MMP-2 values were normalized to total ng of MMP-2 to account for medium volume differences in static and pumped culture settings.

### EEO Emergence Studies

EEOs were thawed into Matrigel and expanded as previously reported. Once the EEOs were lumenized with an average diameter greater than 250 μm, the Matrigel droplets were harvested using cold Cell Recovery Solution (Corning 354253). Briefly, Matrigel droplets were collected with wide-bore pipette tips and mixed with Cell Recovery Solution and placed on ice for approximately 20-30 minutes until the Matrigel was fully dissolved. After dissolving, the solution was centrifuged at 300 g×5 min to get a cell pellet. Organoids were manually broken into single cells using previously described protocols²⁰ in a TrypLE (ThermoFisher 12604021) solution containing DNAase (Sigma Aldrich D4527-40KU) with manual pipetting. Single cells were counted and resuspended in a functionalized PEG solution (all components except crosslinker) at a density of 1000 cells/μL of gel. Next, the LW crosslinker was added at the desired concentration and mixed thoroughly before pipetting 3 μL droplets in a non-TC treated 96 well plate. The hydrogel droplets were placed in a 37° C. incubator for 30 minutes to allow gelation to occur before adding 150 μL of complete EEO medium (including ROCKi). Imaging was performed every day using a Molecular Devices ImageXpress for the course of a week to visualize EEO emergence. Media changes were performed every other day.

### E2 Presoaking

PDMS rapidly absorbed hormones, therefore, to mitigate this issue a method to pre-saturate the devices with estradiol (E2) was developed prior to using them in experiments. To do this, devices were fully submerged in a 10 nM E2 solution in PBS for 24 hours and then the solution was aspirated, and devices were dried in a 37° C. vacuum oven overnight. Controls for this experiment included devices that were not presoaked and devices that were presoaked and did not get media with E2. After presoaking devices (in a solution of 10 nM E2 or PBS without E2) and drying, blank gel without cells was loaded into 3 devices per condition (9 devices total). After gelation, media containing 10 nM E2 or without E2 was added and changed daily for a duration of 6 days. Media was collected each day and stored at −80° C. for downstream analysis. After the experiment, media from each condition was analyzed using an estradiol ELISA kit (DRG International EIA2693). The concentration of estradiol in the spent medium was normalized to the 10 nM E2 media to calculate the fraction of E2 that was taken up by PDMS.

### Image Analysis

Devices were imaged at either Day 5 or 7 on a Zeiss LSM 880 confocal microscope with a 10× objective using a 488 laser to image the GFP-labeled endothelial cells. Z-stacks were taken at a least 3 distinct sites per device and export a maximum intensity projection (MIP) for analysis. Image files were processed using REAVER³⁸, a MATLAB based analysis, to quantify mean network diameter, total network length, and area fraction.

### Perfusability Quantification

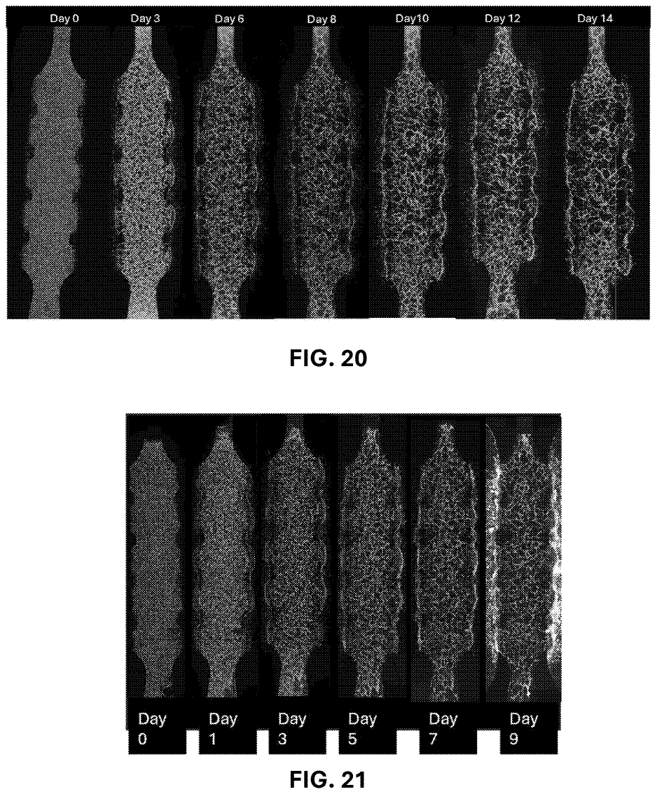

Perfusability was assessed by the flow of fluorescent microbeads which could be added to the media reservoir and circulated for at least an hour on the final day of the experiment, with experiments ending between day 7 and day 10. Since the beads did not fully fill the vessels and flow across the vessels, perfusability was quantified per region of the device. If at least half of the vessels in each region of the device contained beads that section of the device was considered perfusable. Quantification was performed by dividing the device per post region and the 2 edges (FIG. 20), with each post region representing 16.6% (⅙) of the total area and the edges together representing 16.6% (⅙) of the device.

### Quantification and Statistical Analysis

Data were expressed as mean±standard deviation, with individual data replicates shown. Each point represented a single device. Graphs were created and statistical analysis was performed using GraphPad Prism 10. Specific sample sizes were noted on each figure legend. Statistical significance was defined by *p<0.05, **p<0.01, ***p<0.001.

### Results and Discussion

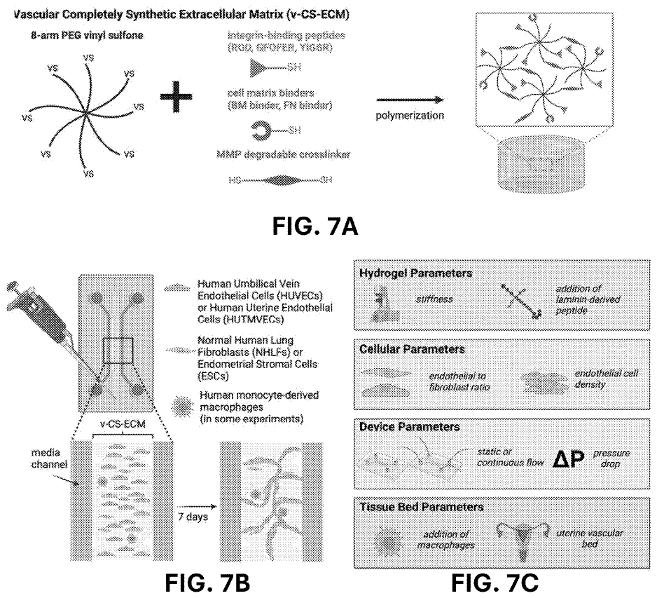

The design approach and testing paradigm for this “vascularizable” CS-ECM (termed here “v-CS-ECM”) (FIG. 7A) was guided by a semi-empirical screen of hydrogel and cellular parameters starting with those governing interconnected endothelial network formation in static microfluidic culture (FIGS. 7B-7C). Following analysis of outcomes in static culture, continuous fluid flow was introduced, and parameters were systematically varied to achieve lumenized endothelial network structures, termed vessels. Finally, this hydrogel was demonstrated to support microvessel co-culture with organoids and macrophages, opening potential for complex disease modeling.

### Semi-Empirical Screen of Cell and Hydrogel Parameters Impacting Vascular Network Formation in Static Culture

### Introduction to Screen and Description of Microfluidic Device

A semi-empirical screen of various hydrogel, cellular, device and tissue bed parameters was used to accomplish microvessel morphogenesis in a CS-ECM hydrogel (FIG. 7C) to create v-CS-ECM, starting with morphogenesis of network structures in static culture, with a standard cell source, then progressing to perfusable vessels in cultures exposed to continuous flow and a tissue-specific cell source. While microvascular phenotypes are well-known to be tissue-specific²⁷, practical issues of accessibility (including of cells already labelled with fluorescent reporters) and wide-spread use in other angiogenesis assays, contributed to the adoption of human umbilical vein endothelial cells (HUVECs) combined with normal human lung fibroblasts (NHLFs) as a workhorse model system for self-assembled microvessel formation^{1,3,5,28}. As the ultimate goal was to develop protocols that can readily be adapted to a range of microvascular cell sources, a strategy of using HUVEC/NHLF in initial studies in screens as a benchmark against established work in the field was implemented, then the protocols were extended to microvasculature cells relevant for endometriosis lesions.

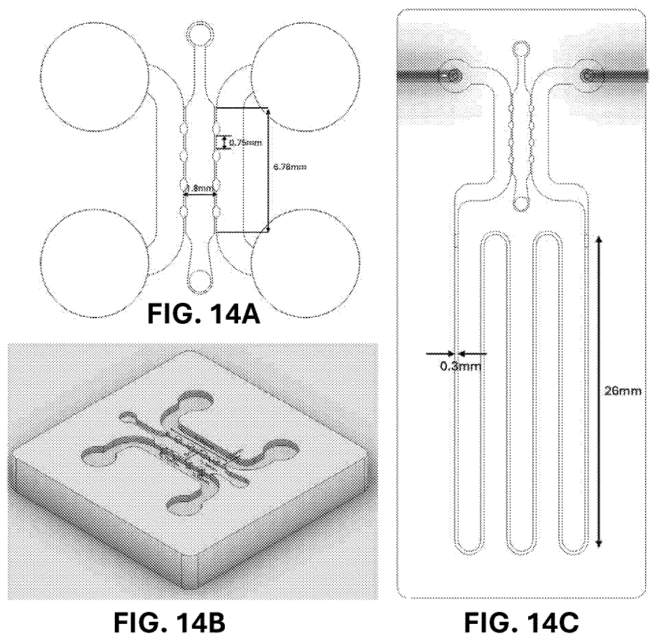

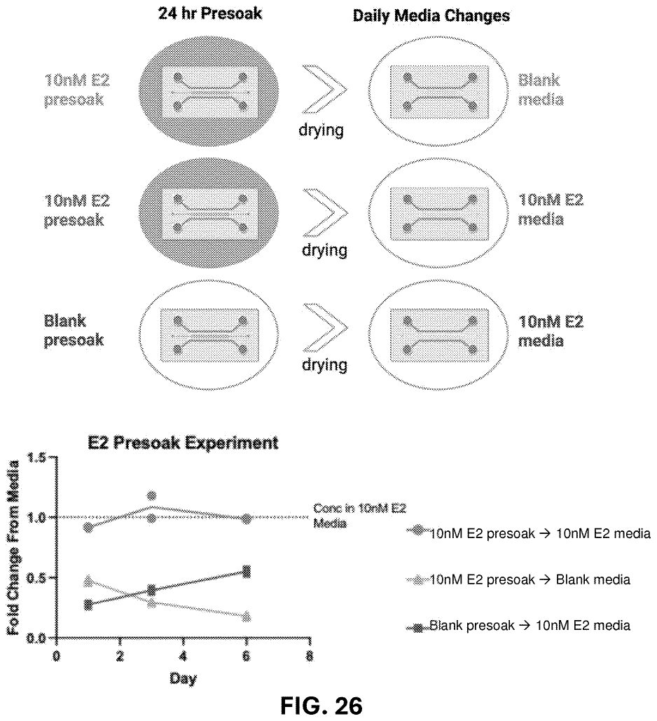

Based on previous protocols described for vascular morphogenesis in microfluidic devices^{3,29,30}, a custom device that could accommodate the anticipated relatively large size of a growing endometriosis lesion was designed and fabricated, with a vascular bed 1.8 mm in width and 0.45 mm in height (see detailed dimensions in FIGS. 14A-14C; for reference, commercial AIM Biotech chips are 1.3 mm wide and 0.25 mm height). Four microposts on each side of the central gel channel pin the liquid hydrogel precursor solution via surface tension during loading. The chips were fabricated from PDMS, with appropriate pre-saturation with hormones where needed (FIG. 26).

### Hydrogel Composition and Biophysical Parameters

A semi-empirical screen of factors influencing endothelial cell network formation in PEG-based CS-ECM determined that endothelial cells favor soft matrices¹⁰. However, an additional constraint is gel swelling post-crosslinking, as formulations that support network formation in free-swelling gels with large swelling coefficients may fail to support network formation in microfluidic devices, where swelling is constrained¹⁰. Therefore, combining these considerations, a softer gel with a smaller swelling factor compared to the canonical regime used for organoids²⁰ was achieved by decreasing the weight percent of PEG and increasing the crosslinking ratio, respectively. Additionally, threshold concentrations of adhesion and matrix-targeting peptides required to support robust organoid and stromal co-culture were kept at 1.5 mM PHSRN-K-RGD, 1.5 mM GFOGER, 0.5 mM BM Binder, 0.5 mM FN Binder^{19,20} while adding a peptide targeted to endothelial interactions.

Laminin, an ECM protein, is an abundant component of the basement membrane in blood vessels^{31,32} Endothelial cells cultured in collagen hydrogels supplemented with laminin exhibit greater end-to-end endothelial network formation and increased VEGF uptake, compared to endothelial cells cultured in collagen alone³³. Short adhesive peptides derived from laminin, including IKVAV from the laminin al chain and YIGSR from the laminin β1 chain are candidates for creating v-CS-ECM³⁴⁻³⁶. YIGSR can regulate endothelial cell morphology in synthetic matrices, and when combined with RGD peptides, increase endothelial cell migration rates and endothelial tube length in vitro^{32,34,35,37} Given that it was desirable to maintain the adhesion and matrix-targeting peptides developed for organoid culture, including RGD, incorporation of YIGSR instead of IKVAV was chosen, as the combination of YIGSR and RGD in PEG hydrogels could form more stable endothelial tubule networks with increased tubule length and diameter compared to PEG hydrogels functionalized with a combination of IKVAV and RGD³⁴.

As a starting point, a softer hydrogel formulation (3.0 wt % PEG, 0.52 thiol-ene crosslinking ratio) was compared to a previous formulation (3.6 wt % PEG, 0.43 thiol-ene crosslinking ratio). These initial experiments to screen hydrogel composition and biophysical parameters were performed with a total cell concentration of 12×10⁶/mL (7:1 HUVEC:NHLF), based on the upper end of the range of results reported in fibrin. Network area fraction, determined by a MATAB-based network quantification pipeline³⁸ of day 5 images (Methods) as a metric that captures significant differences between formulations, was used as network metrics that can be reasonable predictors of later success in forming lumenized, perfusable vessels.

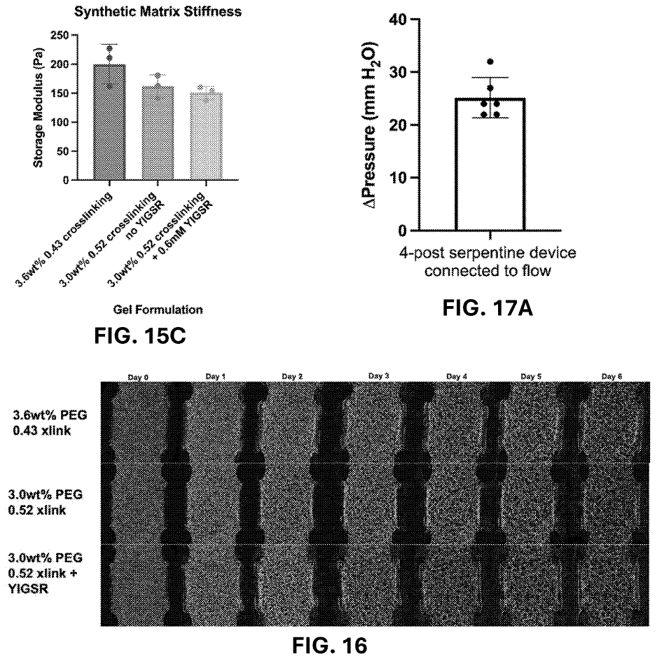

This formulation slightly increased the network area fraction in HUVEC/NHLF seeded into devices (from 0.30 to 0.34; FIG. 8A). Further, this formulation demonstrated a storage modulus of ~180 Pa and a swelling factor of 1.5 (approximately 25% softer, and with 30% less swelling compared to the previous organoid formulation²⁰, FIG. 15). Additionally, when YIGSR was added at a concentration (0.6 mM), while maintaining the concentrations of the four other peptides in the canonical CS-ECM constant, a significant increase in area fraction of endothelial cells compared to the previous organoid formulation²⁰ (from 0.30 to 0.38, p<0.05; FIG. 8A) was achieved. The addition of YIGSR did not change the hydrogel swelling or bulk stiffness (FIG. 15), showing that this increase in endothelial area fraction can likely be attributed to the peptide addition and not other hydrogel parameters.

### Cell Parameters

For the HUVEC/NHLF combination in fibrin, both formation of connected microvessel networks and ultimately perfusable microvessels depends on both the absolute and relative endothelial cell stromal cell concentrations³. Networks formed in the v-CS-ECM formulation described above were not perfusable, with a standard dextran perfusion assay initiated via gravity driven flow after 7 days of static culture. It was therefore speculated that (i) the diameter of vessels formed was too low (<25 μm) to support perfusion and immune cell circulation; and (ii) morphogenesis in v-CS-ECM may require a greater number of endothelial cells compared to the standard concentration range (5-12M HUVECs/mL) needed for fibrin^{3,39}.

To that end, a semi-empirical parameter screen was performed with 12-18M HUVECs/mL and ratios of 5:1, 7:1, and 10:1 endothelial to fibroblast cells. 18M HUVECs/mL with a 7:1 HUVEC:NHLF ratio resulted in the greatest area fraction in static culture conditions and this combination was used for the remainder of studies involving HUVEC-NHLF cocultures (FIGS. 8B, 8C). Notably, when using this concentration and ratio, the v-CS-ECM formulation significantly increased the area fraction of endothelial cells compared to both the softer CS-ECM (0.45 to 0.52, p<0.05) and organoid formulation²⁰ (0.35 to 0.52, p<0.0001) (FIG. 8D, FIG. 16). This condition formed interconnected networks with an area fraction of ~0.5 and a mean network diameter approximately 35 μm; however, despite significant network interconnectivity which is an important first step towards creating vessel-like structures, these networks still did not have open-lumen structures necessary to allow perfusion. It was contemplated that, as in vivo, fluid flow factors may play a crucial role in development of fully lumenized, perfusable vessels.

### Introducing Flow and a Pressure Drop Results in Perfusable Vasculature

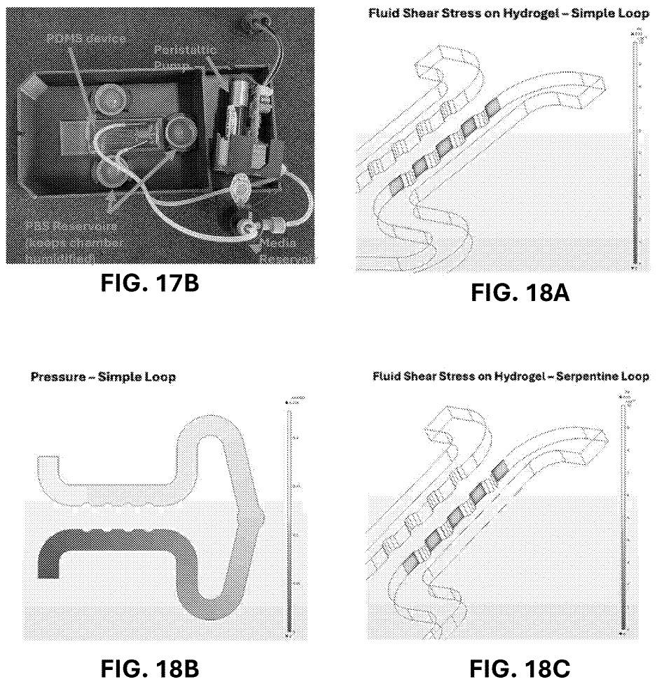

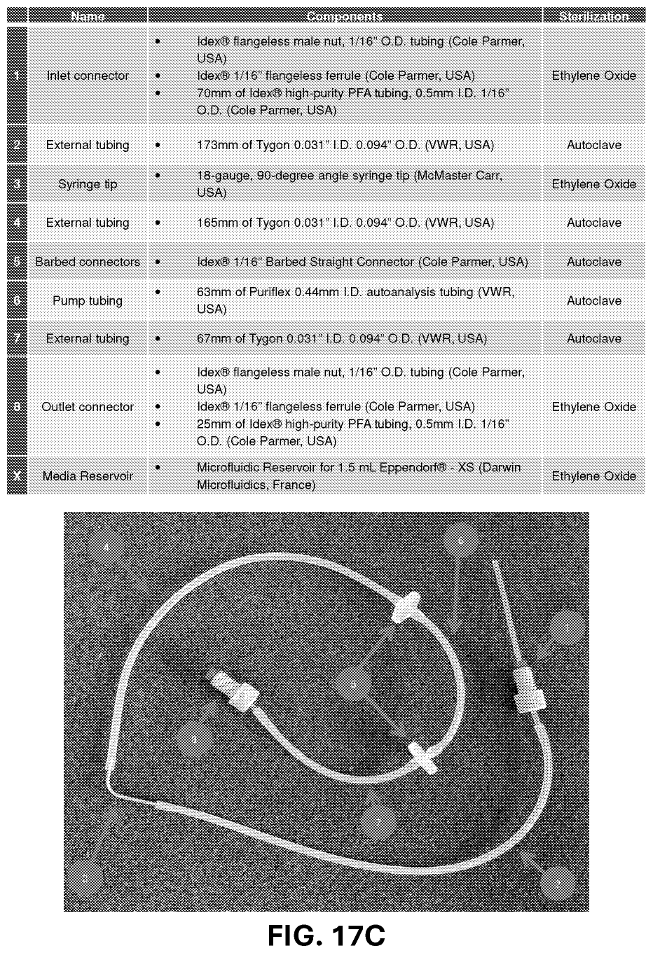

Vessels in vivo are constantly exposed to hemodynamic forces, which influences their remodeling and functions^{9,9,40}. Many investigators use gravity-driven pressure gradients generated by a height difference in media reservoirs feeding the side channels⁴¹. Here, to avoid flow transients associated with gradual equilibration of reservoir heights, the static culture device was adapted to configurations incorporating recirculating peristaltic pump-driven flow through the side channels, using the return channel loop to generate defined weak or strong pressure drops across the tissue compartment, as either a simple loop or a serpentine return (FIG. 9A, FIGS. 17A-17C). In these flow setups, the flow rate was controlled at 1 μL/s, imposing a shear stress on the hydrogel walls of approximately 0.07 dyne/cm² (FIGS. 18A-18D). Importantly, all three device designs (static, simple-loop, serpentine-loop) have the same tissue chamber geometry and dimensions (FIG. 9A, detailed dimensions in FIGS. 14A-14C), isolating continuous flow and pressure difference as the only changing variables.

In the first configuration, a simple loop generating a very small (0.2 mm H₂O) DP was used (FIG. 9A, FIGS. 18A-18D), as this would emphasize the imposition of shear rather than transmural flow. After culturing for 7 days, vessels near the inlet and outlet gel-injection ports displayed open lumens and were perfusable as assessed via the flow of microbeads (FIG. 9C). However, there was not observable open vessel formation along the entire length of the central tissue channel.

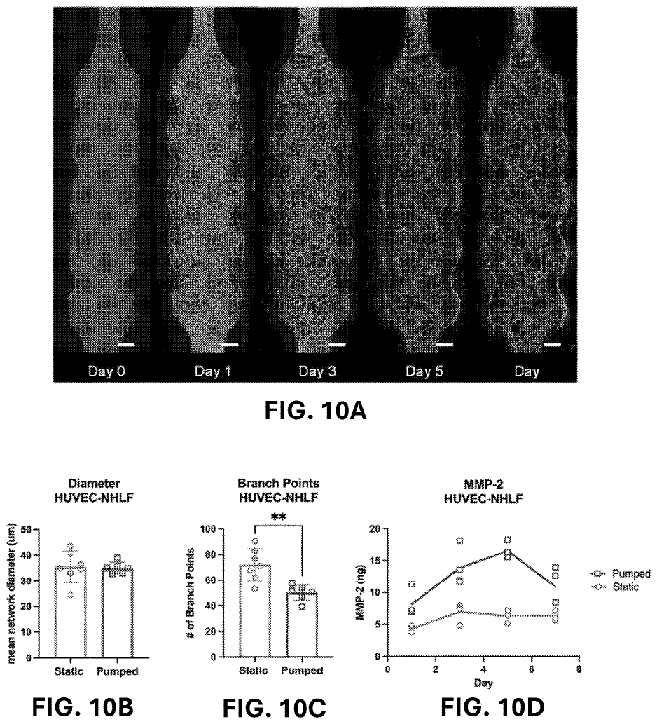

A more significant pressure drop across the channel may increase the fraction of perfusable vessels to facilitate, as the biophysical forces imposed by pressure drops influence endothelial cell physiology^{42,43,41}. To increase the DP in the context of a constant imposed recirculating flow, a serpentine looped channel design was implemented (FIG. 9A). The trans-tissue-channel DP was measured as approximately 25 mm H₂O (FIGS. 17A-17C) close to the value of 32 mm H₂O predicted by a COMSOL model (FIGS. 18A-18D). Notably, when microvessel networks were cultured using the serpentine device exposed to flow throughout the entire duration of the experiment, perfusable vessels were observed, evidenced by the flow of fluorescent microbeads through open-lumen structures across the channel (FIG. 9D, FIG. 10A). The fraction of vessels was approximately 45% (FIG. 9D, FIGS. 19A-19B), with the most perfusable vessels in the top quarter and bottom quarter of the device. Additionally, these vessels could be maintained in culture for at least 14 days under continuous flow (FIG. 20). As an attempt to decrease the high concentration of endothelial cells required, 15M HUVECs/mL (7:1 HUVEC:NHLF ratio) were tested and perfusable vessels in the bottom quarter of the device were observed (FIG. 21), indicating a partial success and likely other parameters that could be changed to decrease the endothelial cell density. While further iteration on parameters such as side channel geometry (influences shear stress on surface endothelia) and post placement (influences tissue biomechanics via its effects on gel compression and on durotaxis) may be needed to reach 100% perfusable vessels across the entire channel, the current findings represent a significant advance in the development of a completely synthetic hydrogel for microvascular morphogenesis. Extensive further iterations were not pursued at this point as applications, such as the endometriosis lesion model, involve additional cell types that will likely influence remodeling and vessel maturation. Both underlying mechanistic hypotheses for differences in performance in these configurations, as well additional phenomenology that might arise from inclusion of additional cell types of interest in applications were then investigated.

### ECM Remodeling Contributes to Perfusable Vessel Formation

MMP-mediated ECM remodeling is one potential flow-induced mechanism contributing to open lumen formation in the synthetic hydrogel between static and continuous flow conditions. MMP-2 facilitates vessel formation in fibrin hydrogels⁴⁴, and the MMP-sensitive crosslinker in v-CS-ECM is susceptible to degradation via MMP-2. MMP-2 concentrations were then measured via ELISA on media samples collected after 24 hr of circulation, at the time of daily media changes, throughout the time course of the morphogenesis process (days 1, 3, 5, and 7). Strikingly, there were significantly higher MMP-2 levels detected via ELISA in the spent medium from networks cultured with continuous flow compared to those networks cultured in static conditions (FIG. 10D). The MMP-2 levels were strikingly higher at days 3 and 5 in the pumped condition compared to static and started to decrease at day 7. The open lumen structures emerging between day 5 and day 7 were observed (FIG. 10A). This enhanced remodeling contributed to the endothelial network to vessel progression. Therefore, this could be a useful marker for evaluating future hydrogel formulations for microvessel formation, as this elevated MMP-2 corresponded with open lumens that were not present in static conditions. Interestingly, in these vessel networks, a discernible difference in network diameter between static and serpentine-looped pumped devices was not observed (35 μm for both static and pumped conditions, FIG. 10B), demonstrating that an increased vessel diameter did not facilitate open-lumen structures. The pumped devices did result in simpler network structures evidenced by significantly less branch points (FIG. 10C, static=72, pumped=50, p<0.01).

Successful Incorporation of Macrophages into Endothelial Networks

While the bedrock of microvascular engineering focuses on co-culture of endothelial cells with a fibroblast population, macrophages are key regulators of vessel sprouting, serve as sources of angiogenic growth factors, and are observed to align with nascent vasculature similar to support cells⁴⁶. Macrophages are phenotypically plastic innate immune cells ubiquitous to tissue microenvironments, where they perform tissue homeostatic functions including lipid and iron processing, along with wound-healing and immune defense against pathogens^{47,48}. The homeostatic tissue-resident macrophage population is augmented when needed by trafficking of circulating CD14+ monocytes into the tissue, driven by chemokine cues such as CCL2^{47,48}, contributing further to inflammatory cascades that influence vessel and tissue function^{49,50}. Macrophages are implicated as primary drivers of endometriosis lesion pathophysiology, and are viewed as a target of therapeutic intervention^{24,51-53}. Protocols for incorporating macrophages were therefore investigated.

To generate macrophages, CD14+ human monocytes were isolated from healthy donors and differentiated over 6 days using 50 ng/mL macrophage-colony stimulating factor. The static culture screen was implemented (described above, and Methods), starting with keeping a constant concentration of HUVECs at 18M/mL, while varying the concentration of macrophages in the supporting cell population (macrophages, NHLFs) from 0-100%, keeping the ratio of endothelial:supporting cells at 7:1 and the total concentration of supporting cells at 2.57M/mL (FIG. 11A).

As the percentage of macrophages increased from 0-100%, the endothelial network diameter, area fraction, and length density decreased (FIG. 11B). Cultures entirely lacking NHLF (100% macrophage in supporting cell population) exhibited scattered network fragments but did not generate fully formed networks. The 9:1 and 6:1 fibroblast:macrophage ratios supported endothelial network diameter and area fraction values comparable to controls with no macrophages. In day 5 imaging (FIG. 11A), macrophages appeared to have heterogeneous spreading morphologies with clear projections.

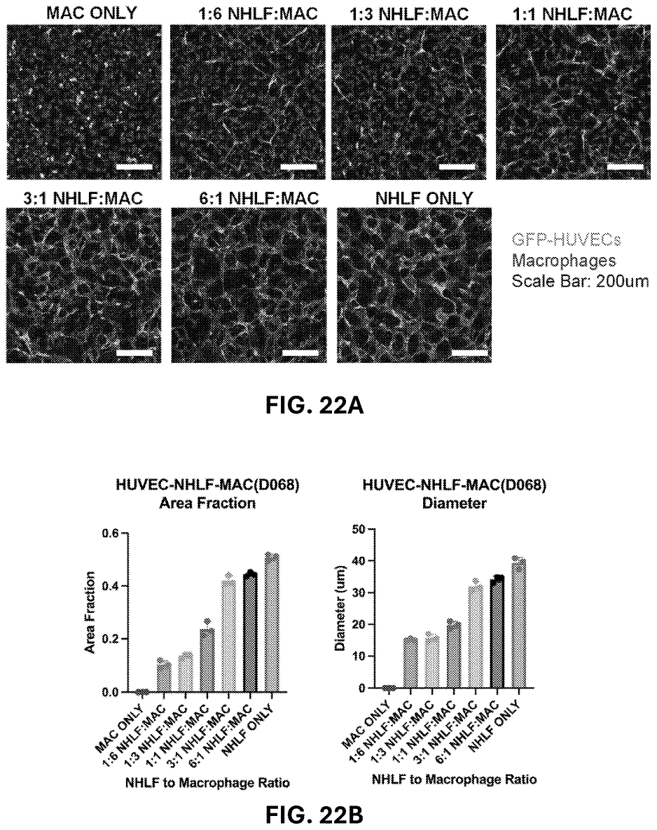

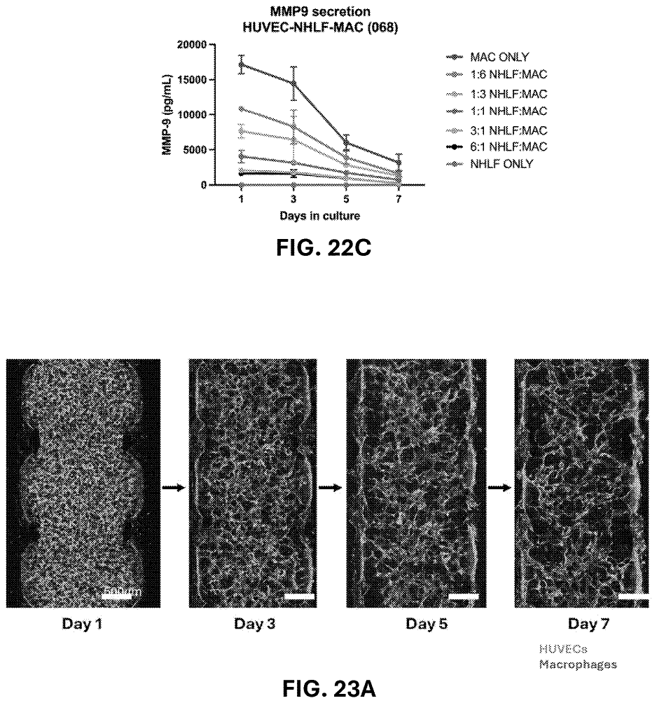

To confirm the functionality of macrophages in the system, media supernatants were analyzed for MMP-9, a major MMP produced by human macrophages that is known to have an influence on angiogenesis^{43,55,56}. The amount of MMP-9 secreted corresponded with the number of macrophages in the culture, and was highest at day 1, with no detectable MMP-9 in the NHLF only condition (FIG. 11C). The endothelial network morphology and MMP-9 trends were repeated with a second macrophage donor, confirming that macrophages maintain functionality in the v-CS-ECM (FIGS. 22A-22C). MMP-2 was analyzed and a clear trend was not observed with the presence of macrophages, except the macrophage only condition had the lowest total secreted enzyme (FIG. 11D).

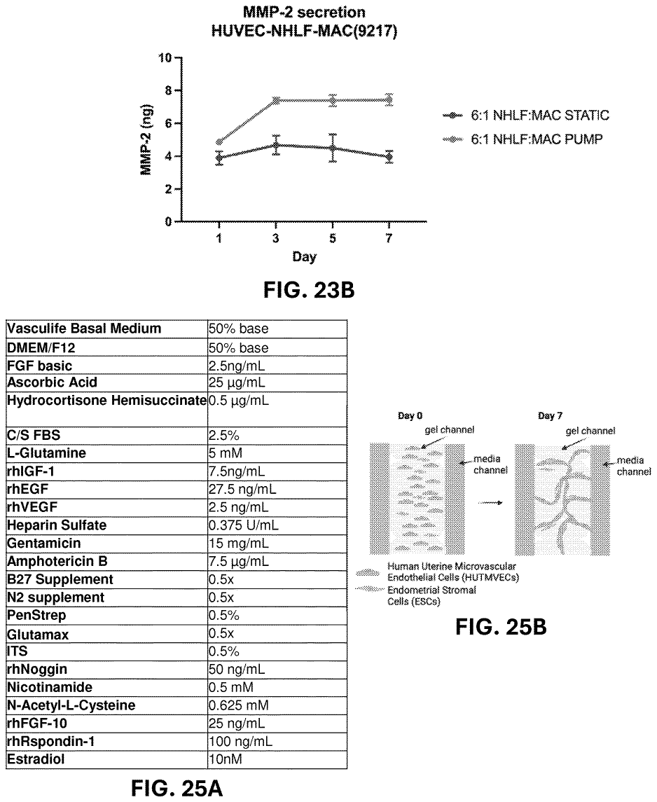

Once it was established that macrophages could be incorporated into microvascular networks, the ability to form perfusable vessels with the 6:1 macrophage ratio was confirmed. Similar to the HUVEC-NHLF only networks, perfusable networks formed in the serpentine loop devices under continuous flow, as evidenced by beads trafficking through the networks (FIG. 11E) and likewise observed the significant increase in MMP-2 secretion when transitioning from static to pumped culture (FIGS. 23A-23B). Close proximity between the macrophages and vessels was observed, likely indicating the macrophages are serving in a supporting cell capacity. Together, this model provides a launch point to study the influence of innate immune cell behavior on vascular networks in a disease-specific context.

Uterine Microvessel Cells Form Perfusable Vessels in v-CS-ECM

While HUVECS and NHLFs are a useful system to benchmark vascular morphogenesis, vascular phenotypes vary significantly between tissues^{27,42,58,59}, prompting experiments to evaluate how well protocols for the benchmark cell types can be extended to microvasculature relevant for a disease-specific model. Adenomyosis is a sibling disease of endometriosis, characterized by endometriosis-like lesions in the myometrium of the uterus. Noting that the fibroblasts in lesions have characteristics of endometrial stromal cells⁶⁰, commercial uterine microvessel endothelial cells were obtained, and they were combined with primary patient-derived endometrial stromal cells (ESCs) as a model for microvasculature in the vicinity of an adenomyosis lesion.

In similar fashion to the initial cell parameter screen with HUVEC and NHLF cell concentrations and ratios (FIGS. 8B-8C), a parameter screen with varying concentrations of endothelial and stromal cells was performed. It was determined 18M HUTMVECs/mL and a ratio of 7:1 result in the greatest network area fraction (0.41) and diameter (31 μm) (FIGS. 12A-12B). When exposing these cultures to continuous flow, vessel structures were perfusable as evidenced by beads trafficking through the open lumens across the device (FIG. 12C). Strikingly, the average vessel diameter was significantly increased (36 μm, FIG. 12D) when upon the transition from static culture to continuous flow conditions. This observation is in contrast to the HUVEC/NHLF case, where the transition from network features observed in static to flow were limited to simpler network structures, without a significant change in vessel diameter (FIGS. 10B-10C).

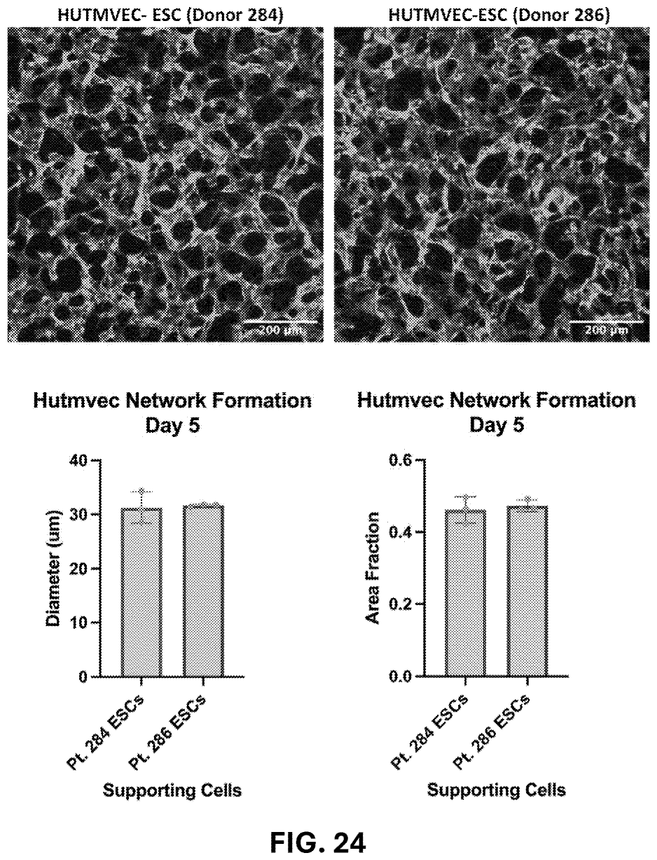

There was a significant increase in MMP-2 detected in the spent media from flow conditions compared to static culture (FIG. 12E), however the relative levels of MMP-2 were much lower for the uterine microvessel cell source than from HUVEC/NHLF. Additionally, there was a continuous increase in MMP-2 throughout the course of a weeklong culture, potentially pointing towards the slightly slower network formation observed via daily imaging with these cells compared to HUVEC-NHLF coculture. These results were repeated with a second ESC donor with no significant changes in outcomes (FIG. 24).

Co-Culture of Endometrial Epithelial Organoids with Uterine Microvascular Networks

Endometriosis lesions are defined as ectopic growth of endometrial-like tissue, typically containing a polarized epithelial monolayer surrounding a fluid-filled lumen with surrounding CD10+ stroma, supported by vasculature and immune cells^{60,62} (FIG. 13A). After demonstrating that the v-CS-ECM can support self-assembly of perfusable vessels formed with uterine-derived endothelial cells and ESCs (FIG. 12), the creation of an endometriosis lesion model was investigated by adding patient-derived endometrial epithelial organoids to the cell population at the time of culture initiation.

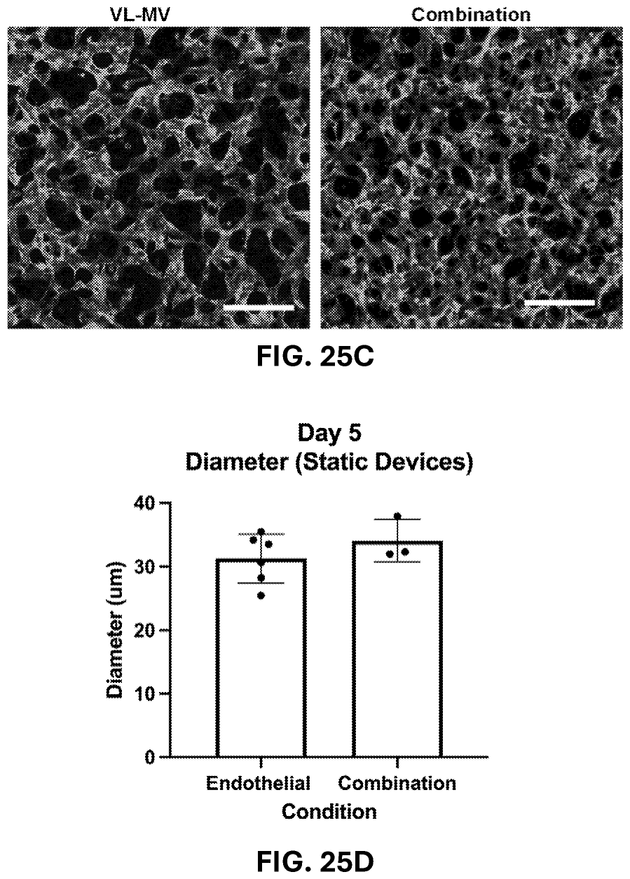

Prior to combining vasculature and organoids, primary endometrial epithelial organoid (EEO) emergence from single cells encapsulated in v-CS-ECM were first assessed using hydrogel droplet culture (FIG. 13B). Similar to the canonical CS-ECM previously described for EEO culture²⁰, the v-CS-ECM supports robust EEO emergence from single cells (FIG. 13C). As a consideration before combining all cell types relevant for co-culture in the full lesion model, a suitable mixed media formulation was developed to support both the EEOs and vasculature. A 50-50 mixture of standard microvascular endothelial media was mixed with the organoid media²⁰ excluding the TGFβ inhibitor A8301 and Rock inhibitor Y-27632²⁰. This media was shown to support network formation in static culture to a comparable extent to standard endothelial cell media (FIGS. 25A-25D). Additionally, endometrial EEOs are typically cultured in an estradiol (E2) containing medium, which is rapidly absorbed into PDMS. To mitigate this, a presoaking method was developed to saturate the PDMS with estradiol and maintain the desired concentration over the duration of the experiment (FIG. 26). For conditions simulating a full menstrual cycle, where hormone concentrations must be cycled, PDMS devices offer formidable challenges and use of relatively non-absorptive thermoplastics is desirable.

Having established that v-CS-ECM supported EEO expansion, patient-derived EEOs approximately 100 μm in diameter were combined with patient-matched ESCs and commercially sourced uterine endothelial cells into the hydrogel precursor solution (FIG. 13D), then injected into a serpentine-looped microfluidic device connected to continuous flow. Over the course of approximately a week, the organoids grew significantly in size as the microvessels self-assembled similar to previous experiments (FIG. 13E, data not shown). This demonstrated the v-CS-ECM and microfluidic device developed in this study can support the coculture of epithelial, stromal, and endothelial cells. Notably, this system facilitated the formation of lesion-like structures and mirrors the structure of in vivo lesions where vessels do not penetrate the lumens of endometriosis lesions but instead surround the fluid-filled epithelial monolayer structures, modeled by EEOs in this system⁶⁰. Therefore, the model is physiologically appropriate when it excludes vessels from within the center of the lesion and has them surrounding the lesion. While the results shown here are simply a proof-of-principle experiment showing this v-CS-ECM facilitates growth of lesion-like structures in the microfluidic devices, this sets a launching point for more developing a better scientific understanding of endometriosis and can be used in the future for testing novel drug compounds.

There has been substantial literature focusing on the use of fibrin and collagen hydrogels to create self-assembled vasculature facilitating foundational studies on vessel biology. Despite significant efforts towards creating self-assembled microvascular networks in synthetic hydrogels, it has been very difficult to achieve microvessels with open lumens which can serve as viable replacements for fibrin or collagen^{5,10,63}. Some success has been achieved within a sprouting angiogenesis microfluidic model, with reports of a dextran-based hydrogel that can support open lumen formation after a 21-day window⁶³⁻⁶⁵. Additionally, hyaluronic acid-based hydrogels supporting endothelial tubules have been reported, with microscopic evidence of lumens and basement membrane structures, however these culture models were not reported in microfluidic devices⁶⁶⁻⁶⁷.

Establishment of a synthetic hydrogel to support perfusable vasculature will further increase the utility of vascularized MPSs in the regulatory space as synthetic PEG-based hydrogels are highly reproducible, modular, and can serve as a blank slate¹¹. The synthetic hydrogel platform was designed specifically to facilitate in vitro modeling of the local pericellular microenvironments each cell type in a disease state experience, as cells can deposit their own ECM and remodel the matrix into a tissue-relevant ECM^{18,20,21}.

In the body, vasculature is subject to fluid flow and mechanical stresses as new vessels are developing and throughout homeostasis^{68,69}. The incorporation of flow to vascularized microphysiological systems further improves the physiological relevance of these model systems, and flow has been shown to play an important role in aiding in vessel formation and maintenance in vitro^{40,44}. Upon introduction of flow and a pressure drop within a normal physiologic range of capillaries and microfluidic models^{41,70}, vessels with open lumens were achieved in the v-CS-ECM in microfluidic devices. Of note, the estimated wall shear stress on the hydrogel is significantly lower than what capillaries experience in vivo (~40 dyn/cm²)⁷¹. Studies will be performed to investigate how increasing the shear stress to a physiologically relevant range would influence vessel formation.

To investigate a potential mechanism to explain why vessels had open lumens when exposed to flow but remained networks in static culture, mechanisms governing extracellular matrix remodeling were examined. The MMP-cleavable crosslinker in the v-CS-ECM is susceptible to degradation by MMP-2 and MMP-9, both of which have known associations with vascular remodeling^{43,44}. Throughout the experiments presented in this study, networks that had been exposed to continuous flow had significantly increased MMP-2 levels in media supernatant collected daily compared to static conditions, and this was observed for both HUVEC-NHLF and HUTMVEC-ESC networks. This finding has also been observed in fibrin hydrogels, with a recent study showing that vessels were unable to form when exposed to an MMP-2 inhibitor, and MMP-2 was upregulated upon exposure to interstitial flow and plays a significant role in vessel formation⁴⁴. Additionally, when macrophages were added into the system it was determined that they secreted MMP-9, which was not detected in HUVEC-NHLF networks without macrophages. This can further contribute to the degradation of the hydrogel, though high concentrations of macrophages limited network formation, and more work is needed to determine exact mechanisms governing perfusable vessel formation in the synthetic ECM.

As more vascularized MPS models have been developed, there are starting to be more tissue-specific endothelial cells in perfusable microvascular models, including mammary microvessels⁴², brain microvessels^{3,72}, and dermal microvessels³. This is the first report of perfusable uterine endothelial cells self-assembled in a microfluidic device, with limited research utilizing uterine-specific endothelial cells^{73,75}. This uterine bed vascular model is being actively used for endometriosis lesion modeling, with the first proof of principle experiments shown here (FIG. 13). This v-CS-ECM will support other tissue-specific endothelial cells, opening avenues for tissue-specific vascularized models.

Overall, this study demonstrated a tool that can be used in vascularized models with room for many future explorations including the combination of organoids and perfusable vasculature for complex MPS. In this study, a development of the first synthetic ECM that supports perfusable self-assembled vascular networks in microfluidic devices was reported. A CS-ECM formulation^{19,20} was modified by decreasing the stiffness and adding YIGSR, a laminin-derived peptide. The number of endothelial cells and supporting fibroblasts to support a robust interconnected endothelial network in static culture conditions was empirically screened before introducing continuous fluid flow. With continuous flow and exposure to a continuous pressure difference across the tissue channel, vasculature with open lumens was achieved in a fully synthetic hydrogel. Additionally, the ability of this hydrogel to support addition of macrophages to endothelial networks and support tissue-specific microvascular endothelial cells was confirmed, setting a launch point for complex disease modeling. Finally, it was demonstrated that this matrix can support the coculture of vasculature and epithelial organoids using endometriosis lesion modeling as a preliminary validation. Overall, this hydrogel should have far reaching impacts on the microfluidic community by creating a reproducible, fully defined matrix to support vascularized tissue models.

### REFERENCES

1. L. Ewald, M., Chen, Y.-H., P. Lee, A. & W. Hughes, C. C. The vascular niche in next generation microphysiological systems. Lab. Chip 21, 3244-3262 (2021).
2. Shirure, V. S., Hughes, C. C. W. & George, S. C. Engineering Vascularized Organoid-on-a-Chip Models. Annu. Rev. Biomed. Eng. 23, 141-167 (2021).
3. Wan, Z. et al. A Robust Method for Perfusable Microvascular Network Formation In Vitro. Small Methods 6, 2200143 (2022).
4. Soto Veliz, D., Lin, K.-L. & Sahlgren, C. Organ-on-a-chip technologies for biomedical research and drug development: A focus on the vasculature. Smart Med. 2, e20220030 (2023).
5. Tronolone, J. J. & Jain, A. Engineering New Microvascular Networks On-Chip: Ingredients, Assembly, and Best Practices. Adv. Funct. Mater. 31, 2007199 (2021).
6. Zhao, N., Pessell, A. F., Zhu, N. & Searson, P. C. Tissue-Engineered Microvessels: A Review of Current Engineering Strategies and Applications. Adv. Healthc. Mater. 13, 2303419 (2024).
7. Murphy, A. R. & Allenby, M. C. In vitro microvascular engineering approaches and strategies for interstitial tissue integration. Acta Biomater. 171, 114-130 (2023).
8. Trietsch, S. J., Israels, G. D., Joore, J., Hankemeier, T. & Vulto, P. Microfluidic titer plate for stratified 3D cell culture. Lab. Chip 13, 3548-3554 (2013).
9. Zhang, S., Kan, E. L. & Kamm, R. D. Integrating functional vasculature into organoid culture: A biomechanical perspective. APL Bioeng. 6, 030401 (2022).
10. Brown, A. et al. Engineering PEG-based hydrogels to foster efficient endothelial network formation in free-swelling and confined microenvironments. Biomaterials 243, 119921 (2020).
11. Caliari, S. R. & Burdick, J. A. A practical guide to hydrogels for cell culture. Nat. Methods 13, 405-414 (2016).
12. Kozlowski, M. T., Crook, C. J. & Ku, H. T. Towards organoid culture without Matrigel. Commun. Biol. 4, 1387 (2021).
13. Lancaster, M. A. & Knoblich, J. A. Organogenesis in a dish: Modeling development and disease using organoid technologies. Science 345, 1247125 (2014).
14. Fatehullah, A., Tan, S. H. & Barker, N. Organoids as an in vitro model of human development and disease. Nat. Cell Biol. 18, 246-254 (2016).
15. Wen, Z., Orduno, M., Liang, Z., Gong, X. & Mak, M. Optimization of Vascularized Intestinal Organoid Model. Adv. Healthc. Mater. 13, 2400977 (2024).
16. Sofman, M., Brown, A., Griffith, L. G. & Hammond, P. T. A modular polymer microbead angiogenesis scaffold to characterize the effects of adhesion ligand density on angiogenic sprouting. Biomaterials 264, 120231 (2021).
17. Valdez, J. et al. On-demand dissolution of modular, synthetic extracellular matrix reveals local epithelial-stromal communication networks. Biomaterials 130, 90-103 (2017).
18. Cook, C. D. et al. Local remodeling of synthetic extracellular matrix microenvironments by co-cultured endometrial epithelial and stromal cells enables long-term dynamic physiological function. Integr. Biol. 9, 271-289 (2017).
19. Hernandez-Gordillo, V. et al. Fully synthetic matrices for in vitro culture of primary human intestinal enteroids and endometrial organoids. Biomaterials 254, 120125 (2020).
20. Gnecco, J. S. et al. Organoid co-culture model of the human endometrium in a fully synthetic extracellular matrix enables the study of epithelial-stromal crosstalk. Med 4, 554-579.e9 (2023).
21. Below, C. R. et al. A microenvironment-inspired synthetic three-dimensional model for pancreatic ductal adenocarcinoma organoids. Nat. Mater. 21, 110-119 (2022).
22. Magno, V., Meinhardt, A. & Werner, C. Polymer Hydrogels to Guide Organotypic and Organoid Cultures. Adv. Funct. Mater. 30, 2000097 (2020).
23. As-Sanie, S. et al. Endometriosis: A Review. JAMA 334, 64-78 (2025).
24. Saunders, P. T. K. & Horne, A. W. Endometriosis: new insights and opportunities for relief of symptoms. Biol. Reprod. ioaf164 (2025) doi:10.1093/biolre/ioaf164.
25. Stadtmauer, D. J. & Wagner, G. P. Single-cell analysis of prostaglandin E2-induced human decidual cell in vitro differentiation: a minimal ancestral deciduogenic signal†. Biol. Reprod. 106, 155-172 (2022).
26. Warner, P. et al. Low dose dexamethasone as treatment for women with heavy menstrual bleeding: A response-adaptive randomised placebo-controlled dose-finding parallel group trial (DexFEM). eBioMedicine 69, 103434 (2021).
27. Barnett, S. N. et al. An organotypic atlas of human vascular cells. Nat. Med. 1-1 (2024) doi:10.1038/s41591-024-03376-x.
28. Wan, Z. et al. A robust vasculogenic microfluidic model using human immortalized endothelial cells and Thy1 positive fibroblasts. Biomaterials 276, 121032 (2021).
29. Whisler, J. A., Chen, M. B. & Kamm, R. D. Control of Perfusable Microvascular Network Morphology Using a Multiculture Microfluidic System. Tissue Eng. Part C Methods 20, 543-552 (2014).
30. Moya, M. L., Hsu, Y.-H., Lee, A. P., Hughes, C. C. W. & George, S. C. In Vitro Perfused Human Capillary Networks. Tissue Eng. Part C Methods 19, 730-737 (2013).
31. Yousif, L. F., Di Russo, J. & Sorokin, L. Laminin isoforms in endothelial and perivascular basement membranes. Cell Adhes. Migr. 7, 101-110 (2013).
32. Grant, D. S. et al. Two different laminin domains mediate the differentiation of human endothelial cells into capillary-like structures in vitro. Cell 58, 933-943 (1989).
33. Stamati, K., Priestley, J. V., Mudera, V. & Cheema, U. Laminin promotes vascular network formation in 3D in vitro collagen scaffolds by regulating VEGF uptake. Exp. Cell Res. 327, 68-77 (2014).
34. Ali, S., Saik, J. E., Gould, D. J., Dickinson, M. E. & West, J. L. Immobilization of Cell-Adhesive Laminin Peptides in Degradable PEGDA Hydrogels Influences Endothelial Cell Tubulogenesis. BioResearch Open Access 2, 241-249 (2013).
35. Fittkau, M. H. et al. The selective modulation of endothelial cell mobility on RGD peptide containing surfaces by YIGSR peptides. Biomaterials 26, 167-174 (2005).
36. Crosby, C. O. & Zoldan, J. Mimicking the physical cues of the ECM in angiogenic biomaterials. Regen. Biomater. 6, 61-73 (2019).
37. Massia, S. P. & Hubbell, J. A. Human endothelial cell interactions with surface-coupled adhesion peptides on a nonadhesive glass substrate and two polymeric biomaterials. J. Biomed. Mater. Res. 25, 223-242 (1991).
38. Corliss, B. A., Doty, R., Matthews, C. & Peirce, S. M. REAVER: An Improved Image Analysis Pipeline for Quantifying Microvascular Networks. FASEB J. 33, 525.7-525.7 (2019).
39. Jeon, J. S. et al. Generation of 3D functional microvascular networks with human mesenchymal stem cells in microfluidic systems. Integr. Biol. Quant. Biosci. Nano Macro 6, 555-563 (2014).
40. Blazeski, A. et al. Engineering microvascular networks using a KLF2 reporter to probe flow-dependent endothelial cell function. Biomaterials 311, 122686 (2024).
41. Chen, S. W. et al. Development of a perfusable, hierarchical microvasculature-on-a-chip model. Lab. Chip 23, 4552-4564 (2023).
42. Moccia, C. et al. Mammary Microvessels are Sensitive to Menstrual Cycle Sex Hormones. Adv. Sci. 10, 2302561 (2023).
43. Sano, H., Watanabe, M., Yamashita, T., Tanishita, K. & Sudo, R. Control of vessel diameters mediated by flow-induced outward vascular remodeling in vitro. Biofabrication 12, 045008 (2020).
44. Zhang, S. et al. Interstitial Flow Promotes the Formation of Functional Microvascular Networks In Vitro through Upregulation of Matrix Metalloproteinase-2. Adv. Funct. Mater. 32, 2206767 (2022).
45. Haase, K. et al. Physiologic flow-conditioning limits vascular dysfunction in engineered human capillaries. Biomaterials 280, 121248 (2022).
46. Moore, E. M. & West, J. L. Harnessing Macrophages for Vascularization in Tissue Engineering. Ann. Biomed. Eng. 47, 354-365 (2019).
47. Chen, S. et al. Macrophages in immunoregulation and therapeutics. Signal Transduct. Target. Ther. 8, 207 (2023).
48. Okabe, Y. & Medzhitov, R. Tissue biology perspective on macrophages. Nat. Immunol. 17, 9-17 (2016).
49. Silberman, J., Jha, A., Ryan, H., Abbate, T. & Moore, E. Modeled vascular microenvironments: immune-endothelial cell interactions in vitro. Drug Deliv. Transl. Res. 11, 2482-2495 (2021).
50. O'Connor, C., Brady, E., Zheng, Y., Moore, E. & Stevens, K. R. Engineering the multiscale complexity of vascular networks. Nat. Rev. Mater. 7, 702-716 (2022).
51. Burns, G. W. et al. Spatial transcriptomic analysis identifies epithelium-macrophage crosstalk in endometriotic lesions. iScience 28, 111790 (2025).
52. Henlon, Y. et al. Single-cell analysis identifies distinct macrophage phenotypes associated with prodisease and proresolving functions in the endometriotic niche. Proc. Natl. Acad. Sci. U.S.A. 121, e2405474121 (2024).
53. Miller, C. M., Germano, D. P. J., Chenoweth, A. M. & Holdsworth-Carson, S. Mathematical modelling of macrophage and natural killer cell immune response during early stages of peritoneal endometriosis lesion onset. J. R. Soc. Interface 22, 20250076 (2025).
54. Moore, E. M., Ying, G. & West, J. L. Macrophages Influence Vessel Formation in 3D Bioactive Hydrogels. Adv. Biosyst. 1, 1600021 (2017).
55. Barillari, G. The Impact of Matrix Metalloproteinase-9 on the Sequential Steps of the Metastatic Process. Int. J. Mol. Sci. 21, 4526 (2020).
56. Lepidi, S. et al. MMP9 production by human monocyte-derived macrophages is decreased on polymerized type I collagen. J. Vasc. Surg. 34, 1111-1118 (2001).
57. Hsu, C.-W. et al. Improved Angiogenesis in Response to Localized Delivery of Macrophage-Recruiting Molecules. PLOS ONE 10, e0131643 (2015).
58. Curtis, M. B., Kelly, N., Hughes, C. C. W. & George, S. C. Organotypic stromal cells impact endothelial cell transcriptome in 3D microvessel networks. Sci. Rep. 12, 20434 (2022).
59. Herron, L. A., Hansen, C. S. & Abaci, H. E. Engineering tissue-specific blood vessels. Bioeng. Transl. Med. 4, e10139 (2019).
60. Colgrave, E. M. et al. Distribution of smooth muscle actin and collagen in superficial peritoneal endometriotic lesions varies from the surrounding microenvironment. Reprod. Biomed. Online 48, 103610 (2024).
61. A. Winkelman, M. et al. Interstitial flow enhances the formation, connectivity, and function of 3D brain microvascular networks generated within a microfluidic device. Lab. Chip 22, 170-192 (2022).
62. Gollbek-Grenda, A. & Olejnik, A. In vitro modeling of endometriosis and endometriotic microenvironment—Challenges and recent advances. Cell. Signal. 97, 110375 (2022).
63. Liu, J. et al. Synthetic extracellular matrices with tailored adhesiveness and degradability support lumen formation during angiogenic sprouting. Nat. Commun. 12, (2021).
64. Trappmann, B. et al. Matrix degradability controls multicellularity of 3D cell migration. Nat. Commun. 8, 371 (2017).
65. Mote, N., Kubik, S., Polacheck, W. J., Baker, B. M. & Trappmann, B. A nanoporous hydrogel-based model to study chemokine gradient-driven angiogenesis under luminal flow. Lab. Chip 24, 4892-4906 (2024).
66. Anderson, A. R. et al. Engineering the Microstructure and Spatial Bioactivity of MAP Scaffolds Instructs Vasculogenesis In Vitro and Modifies Vessel Formation In Vivo. Adv. Funct. Mater. 35, 2400567 (2025).
67. Hanjaya-Putra, D. et al. Controlled activation of morphogenesis to generate a functional human microvasculature in a synthetic matrix. Blood 118, 804-815 (2011).
68. Campinho, P., Vilfan, A. & Vermot, J. Blood Flow Forces in Shaping the Vascular System: A Focus on Endothelial Cell Behavior. Front. Physiol. 11, (2020).
69. Davies, P. F. Flow-mediated endothelial mechanotransduction. Physiol. Rev. 75, 519-560 (1995).
70. Dhadwal, A., Wiggs, B., Doerschuk, C. M. & Kamm, R. D. Effects of anatomic variability on blood flow and pressure gradients in the pulmonary capillaries. J. Appl. Physiol. 83, 1711-1720 (1997).
71. Papaioannou, T. G. & Stefanadis, C. Vascular wall shear stress: basic principles and methods. Hell. J. Cardiol. HJC Hell. Kardiologike Epitheorese 46, 9-15 (2005).
72. Winkelman, M. A. & Dai, G. Bioengineered perfused human brain microvascular networks enhance neural progenitor cell survival, neurogenesis, and maturation. Sci. Adv. 9, eaaz9499 (2023).
73. Gnecco, J. S. et al. Compartmentalized Culture of Perivascular Stroma and Endothelial Cells in a Microfluidic Model of the Human Endometrium. Ann. Biomed. Eng. 45, 1758-1769 (2017).
74. Zambuto, S. G. et al. Endometrial decidualization status modulates endometrial microvascular complexity and trophoblast outgrowth in gelatin methacryloyl hydrogels. Npj Womens Health 2, 1-16 (2024).
75. Zambuto, S. G., Jain, I., Theriault, H. S., Underhill, G. H. & Harley, B. A. C. Cell Chirality of Micropatterned Endometrial Microvascular Endothelial Cells. Adv. Healthc. Mater. n/a, 2303928.
76. Bahlmann, L. C. et al. Targeting tumour-associated macrophages in hodgkin lymphoma using engineered extracellular matrix-mimicking cryogels. Biomaterials 297, 122121 (2023).
77. Datta, A. et al. Axl inhibitor-mediated reprogramming of the myeloid compartment of the in vitro tumor microenvironment is influenced by prior targeted therapy treatment. Front. Immunol. 16, (2025).
78. Poon, C. Measuring the density and viscosity of culture media for optimized computational fluid dynamics analysis of in vitro devices. J. Mech. Behav. Biomed. Mater. 126, 105024 (2022).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

We claim:

1. A formulation for a matrix for culturing cells comprising
a branched poly(alkyl oxide) polymers such as an 8-arm, 20 kDa PEG macromers (“PEG-VS”) synthesized with a tripentaerythritol core and functionalized with vinyl sulfone terminal groups (molar degree of substitution <90%),
thiol-functionalized extracellular matrix polymer, preferably hyaluronic acid (HA-SH), preferably with a molecular weight of approximately 300 kDa and molar degree of substitution of approximately 20-30, preferably thiolated at the carboxylic acid residues of the D-glucuronic acid portion of the repeating units of the polymer, preferably consisting of one D-glucuronic acid group and one N-acetyl-D-glucosamine group.

2. The formulation of claim 1, comprising an aqueous solution, preferably sterile phosphate buffered saline (PBS), pH 7.4 or Dulbecco's phosphate buffered saline and 1 M 4-(2-hydroxyethyl)-1-piperazineethanesulfonic acid (HEPES) pH 7.8-8.2.

3. The formulation of claim 1, comprising monofunctional or bifunctional peptides containing one or two thiol groups or functional groups reactive with or bound to the reactive functional group present on the branched poly(alkyl oxide) species.

4. The formulation of claim 1, wherein the peptides are selected from the group consisting of:
“LW,” a peptide with one cysteine residue at each end to facilitate crosslinking of PEG-VS macromers, containing a MMP-labile domain (GPQGIWGQ) (SEQ ID NO:43) and a Sortase-labile domain (LPRTG) (SEQ ID NO:27);
“PHSRN-K-RGD,” a fibronectin-derived peptide containing the RGD motif and the PHSRN (SEQ ID NO:2) synergy site from the 9th fibronectin type III repeat in a branched configuration;
“GFOGER,” a collagen I-derived peptide presented as a triple helix;
“FN-binder,” a peptide with non-covalent binding affinity for fibronectin; and
“BM-binder,” a peptide with non-covalent binding affinity for collagen IV and laminin.

5. The formulation of claim 1, wherein the polymers are crosslinked to form a matrix.

6. The formulation of claim 5, further comprising cells.

7. A method of making the formulation of claim 1, comprising providing the polyalkylene oxide and extracellular matrix polymers and adhesion ligand peptides and crosslinking.

8. The method of claim 7, further comprising providing cells, optionally wherein the cells are endothelial cells, smooth muscle cells, epithelial cells, and organoid forming cells.

9. A synthetic hydrogel cell culture matrix comprising:
a branched polyalkylene glycol;
one or more integrin peptides; and
one or more extracellular matrix (ECM) binding peptides,
wherein the one or more integrin peptides and the one or more ECM binding peptides are bound to the branched polyalkylene glycol,
wherein the one or more integrin peptides comprise GFOGER (SEQ ID NO:1) and RGD (SEQ ID NO:3), and
wherein the molar ratio of the one or more integrin peptides to the one or more ECM binding peptides is greater than 1:1.

10. The synthetic hydrogel cell culture matrix of claim 9, having a swelling ratio of ≤1.5, a stiffness of ≤150 Pa, and/or a crosslinking ratio of ≥0.52.

11. The synthetic hydrogel cell culture matrix of claim 9, wherein the one or more integrin peptides further comprise YIGSR (SEQ ID NO:45).

12. The synthetic hydrogel cell culture matrix of claim 10, wherein the one or more ECM binding peptides comprise NH₂-GCREISAFLGIPFAEPPMGPRRFLPPEPKKP(Am) (SEQ ID NO:5) and NH₂-GCRE-TLQPVYEYMVGV-COOH (SEQ ID NO:6).

13. The synthetic hydrogel cell culture matrix of claim 10, further comprising a dithiol crosslinking peptide bound to the branched polyalkylene glycol, optionally via a thioether sulfone bond.

14. The synthetic hydrogel cell culture matrix of claim 10, wherein the branched polyalkylene glycol is at a nominal concentration of ≤3 wt %.

15. The synthetic hydrogel cell culture matrix of claim 11, wherein the YIGSR (SEQ ID NO:45) is at a nominal concentration ranging from about 0.1 mM to about 1 mM.

16. A hydrogel precursor composition for synthetic hydrogel cell culture matrix, comprising:
a branched polyalkylene glycol;
one or more integrin peptides, and
one or more extracellular matrix (ECM) binding peptides; and
a carrier,
wherein the one or more integrin peptides and the one or more ECM binding peptides are bound to the branched polyalkylene glycol,
wherein the one or more integrin peptides comprise GFOGER (SEQ ID NO:1), RGD (SEQ ID NO:3), and YIGSR (SEQ ID NO:45), and
wherein the molar ratio of the one or more integrin peptides to the one or more ECM binding peptides is greater than 1:1.

17. The hydrogel precursor composition of claim 16, wherein:
(i) the one or more ECM binding peptides comprise NH₂-GCREISAFLGIPFAEPPMGPRRFLPPEPKKP(Am) (SEQ ID NO:5) and NH₂-GCRE-TLQPVYEYMVGV-COOH (SEQ ID NO:6);
(ii) the branched polyalkylene glycol is at a nominal concentration of ≤3 wt %; and/or
(iii) the YIGSR (SEQ ID NO:45) is at a nominal concentration ranging from about 0.1 mM to about 1 mM.

18. A kit for synthetic hydrogel cell culture matrix comprising:
(a) the hydrogel precursor composition of claim 16;
(b) a dithiol crosslinking peptide or a dithiol hyaluronic acid; and
(c) optionally one or more types of cells.

19. A method for vascularized organogenesis comprising:
(i) incorporating into the hydrogel precursor composition of claim 16 (a) endothelial cells and (b) optionally stromal cells, macrophages, epithelial cells, and/or organoids;
(ii) mixing the endothelial cells-incorporated hydrogel precursor composition with a dithiol crosslinking peptide to form a pre-gel solution; and optionally
(iii) loading the pre-gel solution into a microfluidic device.

20. The method of claim 19, further comprising,
following step (ii) or (iii), (a) maintaining the pre-gel solution at a temperature for a time period sufficient to form a hydrogel; and
following step (a), (b) exposing the hydrogel to a cell culture media at a temperature for a time period sufficient to form a vascular tissue,
optionally wherein the vascular tissue is a vasculature with open lumen, a vascular lesion, uterine bed vasculature, or endometriosis lesion.
